(19) United States
(12) Patent Application Publication
(10) Pub. No.: US 2025/0369058 A1
(43) Pub. Date: Dec. 4, 2025

(54) Viral Diagnostic using CRISPR RNA combinations and Cas13a enzyme

(71) Applicants: The J. David Gladstone Institutes, a testamentary trust established under the Will of J. David, San Francisco, CA (US); The Regents of the University of California, Oakland, CA (US)

(72) Inventors: Gagandeep Renuka Kumar, San Francisco, CA (US); Melanie Ott, Mill Valley, CA (US); Daniel A. Fletcher, Berkeley, CA (US)

(21) Appl. No.: 18/874,191

(22) PCT Filed: Jul. 7, 2023
(86) PCT No.: PCT/US2023/069755

§ 371 Date: Dec. 12, 2024

Related U.S. Application Data
(60) Provisional application No. 63/367,988, filed on Jul. 8, 2022.

(51) Int. Cl. C12Q 1/70; C12N 9/22; C12N 15/11; C12Q 1/34; C12Q 1/6806; C12Q 1/6823

(52) U.S. Cl. CPC C12Q 1/701; C12N 9/226; C12N 15/111; C12Q 1/34; C12Q 1/6806; C12Q 1/6823; C12N 2310/20; G01N 2333/922

(57) ABSTRACT

The present disclosure relates to methods using CRISPR-Cas13 enzyme, complexed with Influenza A or B crRNA guide RNAs to detect and quantify the presence of Influenza A or B RNA in a sample with enhanced specificity and sensitivity. These methods can be used to diagnose Influenza A or B infection, quantify the concentration of Influenza A or B RNA present in a sample, and identify the presence of different Influenza A subtypes or mutations.

### CROSS-REFERENCE TO RELATED APPLICATION

This application claims the benefit of priority to U.S. Provisional Application No. 63/367,988, filed Jul. 8, 2022, the disclosure of which is incorporated by reference herein.

### INCORPORATION BY REFERENCE OF SEQUENCE LISTING

A Sequence Listing is provided herewith as XML file “2349574.xml” created on Jul. 6, 2023 and having a size of 101,264 bytes. The content of the XML file is incorporated by reference herein in its entirety.

### BACKGROUND

Detection of respiratory infections, including SARS-CoV-2 and influenza A and B, is critical for targeting locations and populations that need medical assistance. For example, the estimated U.S. influenza illnesses in the 2019-2020 season was approximately 38 million people. In that same influenza season, approximately 400,000 people were hospitalized and approximately 22,000 died from the disease.

Current respiratory virus diagnostic assays include RT-qPCR nucleic-acid based tests (NATs) that require lab-based equipment and personnel or rapid influenza diagnostics (RIDTs) that detect viral antigens. These assays are not quantitative or multiplexed with other relevant respiratory viruses. These assays are also not appropriate for use by inexperienced or untrained personnel, such as for at home use.

A rapid, easy-to-use detection assay for viral RNA from respiratory body fluid samples is needed for identifying respiratory infections.

### SUMMARY

Described herein are methods, compositions, and devices for detecting and quantifying target viral RNA, such as Influenza A and B, that are faster and more readily deployable in the field than currently available methods and devices. In addition, the methods, compositions, and devices can readily detect and distinguish between strains and variants of the target viral RNA.

Current rapid influenza diagnostic tests (RIDTs) are immunoassays that can identify the presence of influenza A and B viral nucleoprotein antigens in respiratory specimens and display the result in a qualitative way (positive vs. negative). However, RIDTs are known to have limited sensitivity to detect influenza in respiratory specimens compared to time-consuming RT-PCR or viral culture methods. Negative RIDT has the potential for false negative results, especially during peak influenza activity in a community.

The methods described herein can include: (a) incubating a sample suspected of containing Influenza A or B RNA or virus with one or more Cas13 protein, at least one CRISPR guide RNA (crRNA), and at least one reporter RNA for a period of time sufficient to form at least one RNA cleavage product; and (b) detecting reporter RNA cleavage product(s) with a detector. Such methods are useful for detecting whether the sample contains one or more copies of Influenza A or B viral RNA. The methods are also useful for detecting the absence of infection with the virus carrying the target viral RNA. Moreover, the methods and compositions described herein can also readily identify whether a variant or mutant strain of virus carrying the target viral RNA is present in a sample, and what is the variant or mutation.

The methods described herein are useful for diagnosing Influenza infections in a variety of complex biological samples. For example, the samples can include human saliva, sputum, mucus, nasopharyngeal materials, blood, serum, plasma, urine, aspirate, biopsy tissue, or a combination thereof.

### DESCRIPTION OF THE FIGURES

Many aspects of the present disclosure can be better understood with reference to the following drawings. The components in the drawings are not necessarily to scale. Instead, emphasis is placed on clearly illustrating the principles of the present disclosure. Furthermore, components can be shown as transparent in certain views for clarity of illustration only and not to indicate that the illustrated component is necessarily transparent.

FIG. 1A-1B illustrates use of CRISPR-Cas13 and CRISPR guide RNAs (crRNAs) to detect target RNA. FIG. 1A is a schematic diagram illustrating CRISPR-Cas13 detection of target viral RNA using a CRISPR-Cas13 protein that binds CRISPR guide RNAs (crRNA) to form a ribonucleoprotein (RNP) complex. The crRNA targets or guides the CRISPR-Cas13 protein to target viral RNA sequences (e.g., Influenza RNA), where the Cas13 protein is activated to cleave RNA, including the reporter RNA. FIG. 1B is a similar schematic diagram further illustrating a Cas13a:crRNA ribonucleoprotein (RNP) complex binding of target viral RNA, resulting in activation of the Cas13a nuclease (denoted by scissors). Upon target recognition and RNP activation, Cas13a indiscriminately cleaves a quenched-fluorophore RNA reporter, allowing for fluorescence detection as a proxy for Cas13a activation and the presence of target RNA.

FIG. 2 is a schematic diagram illustrating methods for detection of the SARS-CoV-2 RNA genome and fluorescent detection of reporter RNA. CRISPR guide RNAs (crRNA) that can target or bind to SARS-CoV-2 RNA are used. As illustrated, in a first step the CRISPR-Cas13 protein binds CRISPR guide RNAs (crRNA) to form a ribonucleoprotein (RNP) complex. The RNP complex is inactive but, when mixed with the sample to be tested, binding of the RNP complex to the SARS-CoV-2 RNA in the sample activates the Cas13 protein to cut RNA, including reporter RNA molecules added to the assay mixture. Cleavage of the reporter RNA leads to fluorescence, which can be detected by a fluorescence detector.

FIG. 3 illustrates a point-of-care (POC) method for detecting influenza. As illustrated, a sample can be collected (e.g., a patient's saliva, sputum, mucus, or nasopharyngeal sample), the cells and/or viruses in the sample can be lysed to release any viral RNA that may be present, and the RNA from the sample can be mixed with reporter RNAs and a CRISPR-Cas13 protein-crRNA ribonucleoprotein (RNP) complex. Background fluorescence from control reactions can be subtracted and the fluorescence of the sample can be detected. Detection can be by a fluorometer or other suitable device. Such point-of-care detection allows mobilization of medical support and medical personnel.

FIGS. 4A-C shows the detection of influenza strains with specific RNA guides. The Influenza A and Influenza B RNA guides were designed to detect H1N1 or H3N2 strains of Influenza A or Influenza B (FluB). The RNA guides were tested against H1N1, H3N2, FluB target viral RNA, and ribonucleoprotein (RNP) background control with no target viral RNA. FIG. 4A lists signal slope results for different Influenza A and Influenza B RNA guides. As shown in FIG. 4A, the signals from each reaction were measured over two hours and the signal slopes were calculated. Slope ratios were calculated by dividing the slope of a guide RNA+target (i.e. RNP+target viral RNA) reaction by the slope of guide RNA+no target (i.e. RNP control only) reaction. FIG. 4B also lists signal slope results for different Influenza A and Influenza B RNA guides. The comparative slope ratio of the target viral RNA to the RNP control shown in FIG. 4B was obtained by dividing the signal slopes of H1N1, H3N2, or FluB RNA guides by the signal slopes of the RNP control. When the comparative ratio is high (e.g., greater than 1), the guide RNAs employed in the assay mixture detect H1N1, H3N2, or FluB target viral RNA strains more efficiently. But when the comparative ratio is low (e.g., less than 1), the guide RNAs employed in the assay mixture detect the target viral RNA similarly to the RNP control. FIG. 4C lists RNA guide names for H1N1 and H3N2 strains of Influenza A with slope ratios of more than three and the RNA guides for FluB with slope ratios of more than five.

FIGS. 5A-B show the validation and cross-reactivity of Influenza B (FluB) RNA guides against host RNA and nasal swabs. FIG. 5A shows the signal slopes of the RNA guides for FluB having a slope ratio of more than five, as shown in FIG. 4C, as tested against host RNA and nasal swabs. The signals from each reaction were measured over two hours and the signal slopes were calculated. FIG. 5B shows the comparative slope ratio between the target viral RNA and the RNP control as obtained by dividing the signal slopes of the RNA guides for FluB by the signal slopes of the RNP control. FluB RNA guides FluB_cr10 and FluB-cr13 were found to cross-react significantly with nasal swab material that was positive for FluB. FluB_cr12 and FluB-cr14 were found to not cross-react to the same extent with the nasal swab material that was positive for FluB.

FIGS. 6A-B illustrate improved specificity and/or signals obtained for combined guide RNAs FluB_cr12 and FluB-cr14 that were targeted to different viral RNAs. FIG. 6A graphically illustrates that signal slopes from each reaction of target viral RNA for H3N1, H1N1, FluB, or RNP alone with the RNA guides FluB_cr12 alone, FluB-cr14 alone, or FluB_cr12 and FluB-cr14 combined as measured over two hours. FIG. 6B graphically illustrates the comparative slope ratio between the target viral RNA and the RNP control as obtained by dividing the signal slopes of the target viral RNA by the signal slopes of the RNP control. Combining the RNA guides FluB_cr12 and FluB-cr14 improves detection of FluB target viral RNA more than use of these RNA guides separately. Detection of H3N1, H1N1, or RNP alone was not increased by combining the RNA guides FluB_cr12 and FluB-cr14.

FIGS. 7A-B show the validation and cross-reactivity of Influenza A (H1N1 and H3N2 strains) RNA guides against host RNA and nasal swabs. FIG. 7A graphically illustrates the signal slopes from each reaction that were measured over two hours. The RNA guides for Influenza A having a slope ratio of more than three, as shown in FIG. 4C, were included in the test against host RNA and nasal swabs. FIG. 7B graphically illustrates the comparative slope ratio between the RNA guides for Influenza A and the RNP control as obtained by dividing the signal slopes of Influenza A by the RNP control. Influenza A RNA guides identified in the boxes had the best detection of the target viral RNA in the nasal swabs and were selected for a combination experiment shown in FIGS. 8A-B.

FIGS. 8A-B shows the effect of combining the best Influenza A RNA guides of FIG. 7A on Influenza A target viral RNA detection. FIG. 8A shows the signal slopes for combination of seven Influenza A RNA guides (the “7 g”: cr04m, cr08, cr13, cr16, cr17, cr21, cr22) and four Influenza RNA guides (the “4 g”: cr08, cr16, cr21, cr22) that were tested against target viral RNA for Influenza A (strains H1N1 and H3N2). The signals from each reaction as measured over two hours. FIG. 8B shows the signal slopes of the RNA guides for Influenza A. The signals were divided by the signal slopes of the RNP control determine a comparative slope ratio between the target viral RNA and the RNP control. The slope ratios for the Influenza A RNA guides in the 7 g group were 4.9 and 26.5 for the target viral RNA for H1N1 and H3N2, respectively. The slope ratios for the Influenza A RNA guides in the 4 g group were 4.5 and 26.4 for the target viral RNA for H1N1 and H3N2, respectively. The slope ratios for each of the 7 g and 4 g RNA guide groups were significantly higher than for any of the Influenza A RNA guides alone.

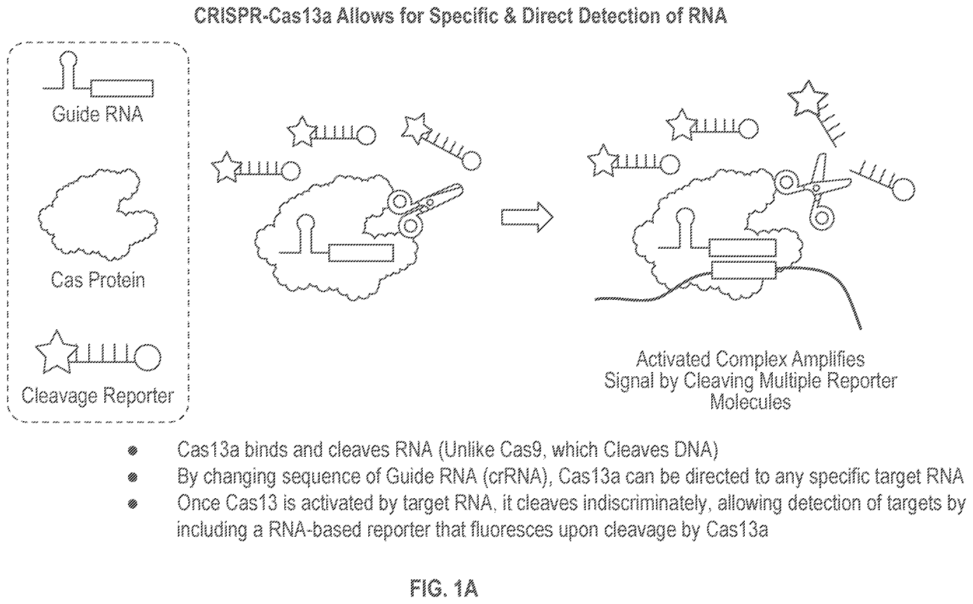

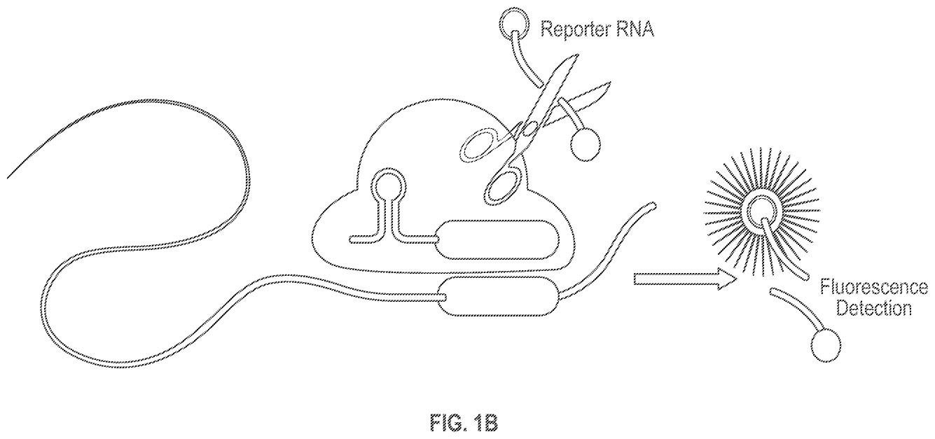

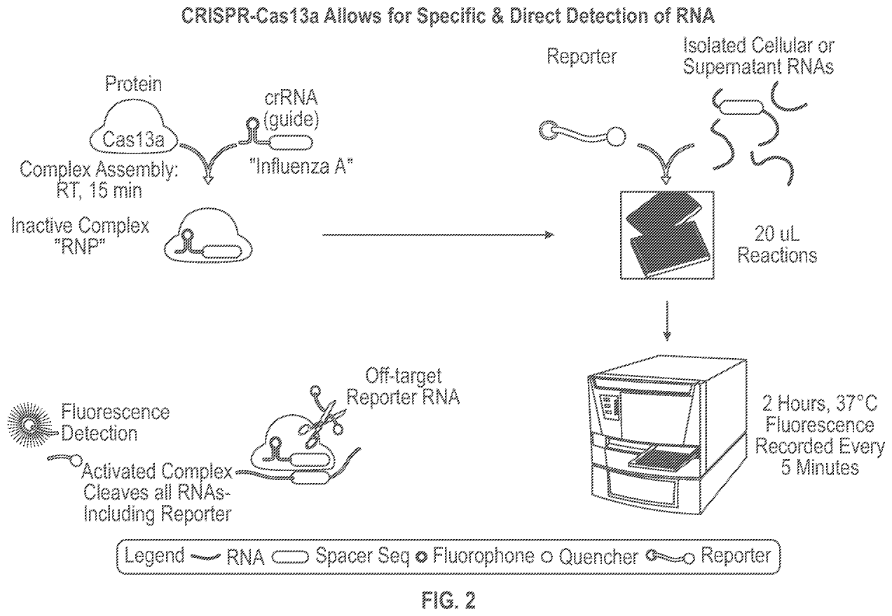

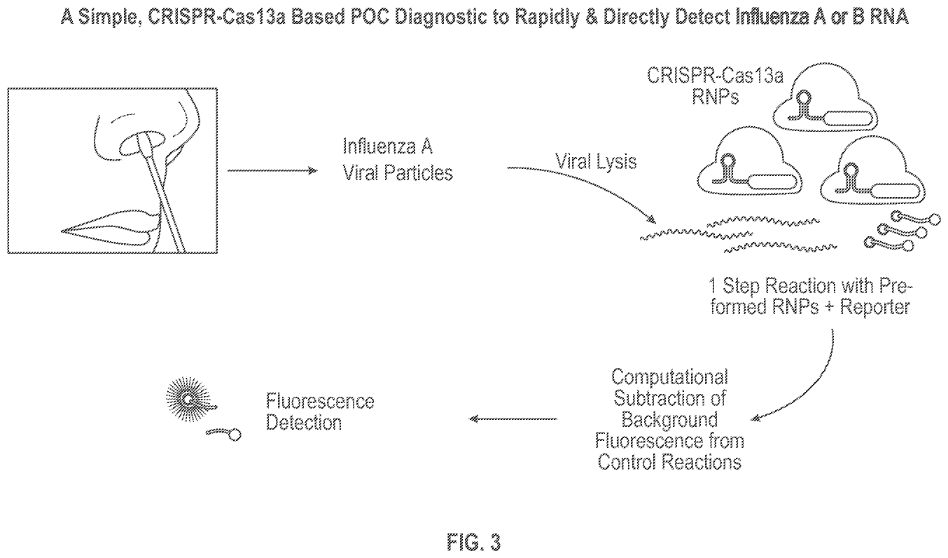

### DETAILED DESCRIPTION

Methods, kits and devices are described herein for rapidly detecting and/or quantifying Influenza virus infection. The methods can include (a) incubating a sample suspected of containing RNA or virus with one or more Cas13 protein, at least one CRISPR guide RNA (crRNA) that binds a target site in at least one of an Influenza A or Influenza B nucleic acid, and at least one reporter RNA for a period of time sufficient to form at least one RNA cleavage product(s); and (b) detecting level(s) of reporter RNA cleavage product(s) with a detector. Such methods are useful for detecting whether the sample contains one or more copies of an Influenza RNA. The methods are also useful for detecting the absence of an Influenza infection.

In some aspects, the disclosure provides methods for identifying the target virus RNA from a sample suspected of containing the target viral RNA. The target virus RNA can be from any RNA virus selected for detection in a sample. In some aspects, the target viral RNA can be from a virus that causes a respiratory infection or establishes its primary infection in the tissues and fluids of the upper respiratory tract. For example, the RNA virus can be an Influenza virus, such as Influenza A or B. Influenza is an enveloped, single stranded RNA virus that recognizes and binds to N-acetylneuraminic (sialic) acid on a host cell surface, including human tracheal epithelial and respiratory epithelium cells. Influenza A is the primary cause of flu epidemics. The target virus RNA can be the RNA from any of Influenza's 18 distinct subtypes of hemagglutinin and 11 distinct subtypes of neuraminidase.

In addition to influenza viruses, the target viral RNA can be common cold coronaviruses, such as strains NL63, OC43, or 229E. The target viral RNA can also be SARS-CoV-2, a hepatitis virus (e.g., HCV), or respiratory syncytial virus (RSV). In some cases, the target viral RNA can be from the human immunodeficiency virus (HIV). The methods can thus be used to detect and identify a combination of viral RNAs, for example, using methods and components described in any of PCT publications WO 2020/051452; WO 2021/188830; and WO 2022/046706, each of which is incorporated by reference herein in its entirety.

In some aspects provided herein are methods for diagnosing the presence or absence of an Influenza infection comprising incubating a mixture comprising a sample suspected of containing Influenza RNA, a Cas13 protein, at least one CRISPR guide RNA (crRNA), and a reporter RNA for a period of time to form any reporter RNA cleavage product(s) that may be present in the mixture; and detecting level(s) of reporter RNA cleavage product(s) that may be present in the mixture with a detector. In some cases, the Influenza RNA in a sample and/or the RNA cleavage products are not reverse transcribed prior to the detecting step. The presence or absence of an Influenza infection in patient is detected by qualitatively or quantitatively detecting level of reporter RNA cleavage product(s) that may be present in the mixture.

The methods described herein have various advantages. For example, the methods described herein can directly detect RNA without additional manipulations. No RNA amplification is generally needed, whereas currently available methods (e.g., SHERLOCK) require RNA amplification to be sufficiently sensitive. The methods, kits, and devices described herein are rapid, providing results within 30 minutes. Expensive lab equipment and expertise is not needed. The methods described herein are amenable to many different sample types (blood, nasal/oral swab, etc.). The methods, kits, and devices described herein are easily deployable in the field (airport screenings, borders, resource poor areas) so that potentially infected people will not need to go to hospitals and clinics where non-infected patients, vulnerable persons, and highly trained, urgently needed medical people may be. Hence, testing can be isolated from facilities needed for treatment of vulnerable populations and from trained personnel needed for urgent and complex medical procedures.

CRISPR-Cas13 is a viable alternative to conventional methods of detecting and quantifying RNA by RT-PCR. The advantages of using CRISPR-Cas13 can be leveraged for Influenza diagnostics. The Cas13 protein targets RNA directly, and it can be programmed with crRNAs to provide a platform for specific RNA sensing. By coupling Cas13 protein to an RNA-based reporter, the collateral or non-specific RNase activity of the Cas13 protein can be harnessed for Influenza detection.

In 2017 and 2018, the laboratory of Dr. Feng Zhang reported a Cas13-based detection system that reached attomolar and zeptomolar sensitivity in detecting Zika virus, but it included an additional reverse transcription step for isothermal amplification of Zika virus cDNA, which was ultimately back-transcribed into RNA for RNA-based detection, a method referred to as SHERLOCK (Specific High Sensitivity Enzymatic Reporter UnLOCKing) (Gootenberg et al. Science 356(6336):438-42 (2017); Gootenberg et al. Science 360(6387): 439-44 (2018)). Although this method improved the sensitivity of Cas13, it introduced two unwanted steps involving reverse transcription and in vitro transcription, which minimizes its potential as a field-deployable and point-of-care device.

The present disclosure provides methods and compositions for diagnosing Influenza infections, quantifying Influenza RNA concentrations, and identifying the presence of different Influenza A subtypes and/or mutations.

In some cases, the methods can be performed in a single tube, for example, the same tube used for collection and RNA extraction. This method provides a single step point of care diagnostic method. In other cases, the methods can be performed in a two-chamber system. For example, the collection swab containing a biological sample can be directly inserted into chamber one of such a two chamber system. After agitation, removal of the swab, and lysis of biological materials in the sample, the division between the two chambers can be broken or removed, and the contents of the first chamber can be allowed to flow into the second chamber. The second chamber can contain the Cas13 protein, the selected crRNA(s), and the reporter RNA so that the assay for Influenza can be performed.

Chamber one can contain a buffer that would facilitate lysis of the viral particles and release of genomic material. Examples of lysis buffers that can be used include, but are not limited to PBS, commercial lysis buffers such as Qiagen RLT+ buffer or Quick Extract, DNA/RNA Shield, various concentrations of detergents such as Triton X-100, Tween 20, NP-40, or Oleth-8, or combinations of such reagents.

Following agitation and subsequent removal of the swab, the chamber may be briefly (e.g., 2-5 mins) heated (e.g., 55° C. or 95° C.) to further facilitate lysis. Then, the division between the two chambers would be broken or removed, and the nasal extract buffer would be allowed to flow into and reconstitute the second chamber, which would contain the lyophilized reagents for the Cas13 assay (Cas13 RNPs and reporter RNA molecules).

Use of such assay tubes can provide single step point of care diagnostic methods and devices.

The methods, devices and compositions described herein for diagnosing Influenza infection can involve incubating a mixture having a sample suspected of containing Influenza RNA, a Cas13 protein, at least one CRISPR RNA (crRNA), and a reporter RNA for a period of time to form reporter RNA cleavage products that may be present in the mixture and detecting a level of any such reporter RNA cleavage products with a detector. The detector can be a fluorescence detector such as a short quenched-fluorescent RNA detector, or Total Internal Reflection Fluorescence (TIRF) detector.

### Reporter:

A single type of reporter RNA can be used. The reporter RNA can be configured so that upon cleavage by the Cas13 protein, a detectable signal occurs. For example, the reporter RNA can have a fluorophore at one location (e.g., one end) and a quencher at another location (e.g., the other end). In another example, the reporter RNA can have an electrochemical moiety (e.g., ferrocene, or dye), which upon cleavage by a Cas13 protein can provide electron transfer to a redox probe or transducer. In another example, the reporter RNA can have a reporter dye, so that upon cleavage of the reporter RNA the reporter dye is detected by a detector (e.g., spectrophotometer). In some cases, one end of the reporter RNA can be bonded to a solid surface. For example, a reporter RNA can be configured as a cantilever, which upon cleavage releases a signal. However, in other cases, a signal may be improved by use of an unattached reporter RNA (e.g., not covalently bond to a solid surface). A surface of the assay vessel or the assay material can have a detector for sensing release of the signal. The signal can be or can include a light signal (e.g., fluorescence or a detectable dye), an electronic signal, an electrochemical signal, an electrostatic signal, a steric signal, a van der Waals interaction signal, a hydration signal, a Resonant frequency shift signal, or a combination thereof.

The reporter RNA can, for example, be at least one quenched-fluorescent RNA reporter. Such quenched-fluorescent RNA reporter can optimize fluorescence detection. The quenched-fluorescent RNA reporters include an RNA oligonucleotide with both a fluorophore and a quencher of the fluorophore. The quencher decreases or eliminates the fluorescence of the fluorophore. When the Cas13 protein cleaves the RNA reporter, the fluorophore is separated from the associated quencher, such that a fluorescence signal becomes detectable.

One example of such a fluorophore quencher-labelled RNA reporter is the RNaseAlert (IDT). RNaseAlert was developed to detect RNase contaminations in a laboratory, and the substrate sequence is optimized for RNase A species. Another approach is to use lateral flow strips to detect a FAM-biotin reporter that, when cleaved by Cas13, is detected by anti-FAM antibody-gold nanoparticle conjugates on the strip. Although this allows for instrument-free detection, it requires 90-120 minutes for readout, compared to under 30 minutes for most fluorescence-based assays (Gootenberg et al. Science. 360(6387):439-44 (April 2018)).

The sequence of the reporter RNA can be optimized for Cas13 cleavage. Cas13 preferentially exerts RNase cleavage activity at exposed uridine or adenosine sites, depending on the Cas13 homolog. There are also secondary preferences for highly active homologs. The inventors have tested 5-mer homopolymers for all ribonucleotides. Based on these preferences, various RNA oligonucleotides, labeled at the 5′ and 3′ ends of the oligonucleotides using an Iowa Black Quencher (IDT) and FAM fluorophore, and systematically test these sequences in the trans-ssRNA cleavage assay as described in the Examples. The best sequence can be moved into the mobile testing.

The fluorophores used for the fluorophore quencher-labelled RNA reporters can include Alexa 430, STAR 520, Brilliant Violet 510, Brilliant Violet 605, Brilliant Violet 610, or a combination thereof.

### Detection:

Various mechanisms and devices can be employed to detect fluorescence. In some cases, the detector is a fluorescence detector, optionally a short quenched-fluorescent RNA detector, or Total Internal Reflection Fluorescence (TIRF) detector. For example, the fluorescence detector can detect fluorescence from fluorescence dyes such the Alexa 430, STAR 520, Brilliant Violet 510, Brilliant Violet 605, Brilliant Violet 610, or a combination thereof.

Some mechanisms or devices can be used to help eliminate background fluorescence. For example, reducing fluorescence from outside the detection focal plane can improve the signal-to-noise ratio, and consequently, the resolution of signal from the RNA cleavage products of interest. Total internal reflection fluorescence (TIRF) enables very low background fluorescence and single molecule sensitivity with a sufficiently sensitive camera. In some cases, mobile phones can be used for detection of Influenza.

In some cases, both Cas13 and reporter RNA can be tethered to a solid surface, upon addition of crRNA and Influenza RNA samples, an activated Cas13 can generate small fluorescent spots on the solid surface when imaged using Total Internal Reflection Fluorescence (TIRF). To optimize this embodiment, the fluorophore side of reporter RNA is tethered to the solid surface as well so that cleavage permits the quencher portion of the reporter RNA to diffuse away. The Cas13 protein can be tethered to the solid surface with a tether that is long enough to allow it to cleave multiple RNA reporter molecules. Counting the bright spots emerging on the solid surface the viral load can be quantified. Use of TIRF in the portable system facilitates detection and reduces background so that the RNA cleavage product signals can readily be detected.

In some cases, a ribonucleoprotein (RNP) complex of the Cas13 protein and the crRNA can be tethered to the solid surface. The crRNA would then not need to be added later. Instead, only the sample suspected of containing Influenza RNA would need to be contacted with the solid surface.

In some cases, the methods described herein can include direct detection of the target RNA in the sample, without performing further sample preparation steps prior to detection, such as depleting a portion of the sample of protein, enzymes, lipids, nucleic acids, or a combination thereof or inactivating nucleases. However, the methods described herein can include depleting a portion of the sample prior to other step(s) or inhibiting a nuclease in the sample prior to the other step(s). For example, the sample can be depleted of protein, enzymes, lipids, nucleic acids, or a combination thereof. In some cases, the depleted portion of the sample is a human nucleic acid portion. However, RNA extraction of the sample is preferably not performed.

In some cases, the methods can include removing ribonuclease(s) (RNase) from the sample. In some cases, the RNase is removed from the sample using an RNase inhibitor and/or heat.

In some cases, the Cas13 protein and/or the crRNA can be lyophilized prior to incubation with the sample. In some cases, the Cas13 protein, the crRNA, and/or the reporter RNA is lyophilized prior to incubation with the sample.

### Sample:

In some embodiments, a biological sample is isolated from a patient. Non-limiting examples of suitable biological samples include saliva, sputum, mucus, nasopharyngeal samples, blood, serum, plasma, urine, aspirate, and biopsy samples. Thus, the term “sample” with respect to a patient can include RNA. Biological samples encompass saliva, sputum, mucus, and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, washed, or enrichment for certain cell populations. The definition also includes sample that have been enriched for particular types of molecules, e.g., RNAs. The term “sample” encompasses biological samples such as a clinical sample such as saliva, sputum, mucus, nasopharyngeal samples, blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived from cells and/or viruses (e.g., from infected cells). A sample containing RNAs can be obtained from such cells (e.g., a cell lysate or other cell extract comprising RNAs). A sample can comprise, or can be obtained from, any of a variety of bodily fluids (e.g., saliva, mucus, or sputum), cells, tissues, organs, or acellular fluids.

In some embodiments, the biological sample is isolated from a patient known to have or suspected to have an Influenza infection. In other embodiments, the biological sample is isolated from a patient not known have an Influenza infection. In other embodiments, the biological sample is isolated from a patient known to have, or suspected to not have, an Influenza infection. In other words, the methods and devices described herein can be used to identity subjects that have an Influenza infection and to confirm that subjects do not have an Influenza infection.

In some cases, it may not be known whether the biological sample contains RNA. However, such biological samples can still be tested using the methods described herein. For example, biological samples can be subjected to lysis, RNA extraction, incubation with Cas13 and crRNAs, etc. whether or not the sample actually contains RNA, and whether or not a sample contains Influenza RNA.

Pre-incubation of the crRNA and Cas13 protein without the sample is preferred, so that the crRNA and the Cas13 protein can form a complex. In some cases, the reporter RNA can be present while the crRNA and the Cas13 protein form a complex. However, in other cases, the reporter RNA can be added after the crRNA and the Cas13 protein already form a complex. Also, after formation of the crRNA/Cas13 complex, the sample RNA (e.g., Influenza RNA) can then be added. The sample RNA (e.g., Influenza RNA) acts as an activating RNA. Once activated by the activating RNA, the crRNA/Cas13 complex becomes a non-specific RNase to produce RNA cleavage products that can be detected using a reporter RNA, for example, a short quenched-fluorescent RNA.

For example, the Cas13 and crRNA are incubated for a period of time to form the inactive complex. In some cases, the Cas13 and crRNA complexes are formed by incubating together at 37° C. for 30 minutes, 1 hour, or 2 hours (for example, 0.5 to 2 hours) to form an inactive complex. The inactive complex can then be incubated with the reporter RNA. One example of a reporter RNA is provided by the RNase Alert system. The sample Influenza RNA can be a ssRNA activator. The Cas13/crRNA with the Influenza RNA sample becomes an activated complex that cleaves in cis and trans. When cleaving in cis, for example, the activated complex can cleave Influenza RNA. When cleaving in trans, the activated complex can cleave the reporter RNA, thereby releasing a signal such as the fluorophore from the reporter RNA.

CRISPR Guide RNA (crRNA):

A CRISPR guide RNA system can be adapted for use in the methods and compositions described herein. The guide RNAs can include: a CRISPR RNA (crRNA or spacer), which can be a 17-20 nucleotide sequence complementary to the target DNA, and a trans-activating crRNA (tracrRNA or stem) that is a binding scaffold for the Cas nuclease. In some cases, the two RNAs are fused to make a single guide RNA (sgRNA). The tracrRNA forms a stem loop that is recognized and bound by the Cas nuclease. The term “guide RNA” as used herein refers to either a single guide RNA (sgRNA) or a crRNA (spacer). The CRISPR technique is generally described, for example, by Mali et al. Science 339:823-6 (2013); which is incorporated by reference herein in its entirety.

In some cases, the at least one CRISPR guide RNA (crRNA) has a sequence with at least 95% sequence identity to any of SEQ ID NOs: 1-37, shown below. In some cases, at least one CRISPR guide RNA (crRNA) has a sequence such as any of SEQ ID NOs: 1-37 or in some cases the crRNA(s) can include those with SEQ ID NOs: 4, 8, 13, 16, 17, 21, 22, 32, or 34-36, or a combination thereof. In some cases, the sample can be incubated with one or two or more crRNAs. For example, the sample can be incubated with at least two, or at least three, or at least four, or at least five, or at least six, or at least seven, or at least eight, or at least nine, or at least nine, or at least ten, or more crRNAs. In some cases, the at least one crRNA has at least about 70%, at least about 75%, at least about 80%, at least about 85%, at least about 90%, at least about 95%, at least about 96%, at least about 97%, at least about 98%, at least about 99%, or about 100%, sequence identity to any SEQ ID NO: 1-37.

In various examples of crRNA(s) that can be used for detection of Influenza B, the crRNA(s) can include those with SEQ ID NOs: 32, 34, 35, 36, or a combination thereof. In some cases, SEQ ID NOs: 34 and 36 can be combined to improve detection of Influenza B.

In various examples of crRNA(s) that can be used for detection of Influenza A, the crRNA(s) can include those with SEQ ID NOs: 4, 8, 13, 16, 17, 21, or 22, or a combination thereof. In some cases, the crRNA(s) can include those with SEQ ID NOs: 8, 16, 21, and 22.

The amount of reporter RNA cleavage product detected is directly correlated with the amount of the target viral RNA. In some cases, the target viral RNA cleavage product concentration can be quantified or determined by use of a standard curve of the reporter RNA cleavage product(s).

At least one crRNA can bind to a region in any of the eight single stranded RNAs of the Influenza RNA genome. In some cases, the region is a single stranded region of the Influenza RNA genome. In other cases, the region is a secondary structure in regions of the Influenza genome with low viral ribonucleoprotein binding.

In some cases, the crRNAs can include additional sequences such as spacer sequences. Table 1 provides examples of Influenza crRNA sequences.

Table 1: Examples of Influenza A and B crRNA Sequences

TABLE 1
Examples of Influenza A and B crRNA Sequences

| SEQ ID NO | Name | Full crRNA (stem + TARGET) |
|---|---|---|
| SEQ ID NO: 1 | FluA_cr01m | gaccaccccaaaaaugaaggggacuaaaacGAGCCGAACGG |
| | | CUGCAUUGA |
| SEQ ID NO: 2 | FluA_cr02m | gaccaccccaaaaaugaaggggacuaaaacCUGGACAGUGG |
| | | UGAACAGUA |
| SEQ ID NO: 3 | FluA_cr03m | gaccaccccaaaaaugaaggggacuaaaacCCUGAAGUCUG |
| | | CUUAAAGUG |
| SEQ ID NO: 4 | FluA_cr04m | gaccaccccaaaaaugaaggggacuaaaacAAUUUUUCCCU |
| | | AGUAGUUCA |
| SEQ ID NO: 5 | FluA_cr05m | gaccaccccaaaaaugaaggggacuaaaacCCCUAGUAGUU |
| | | CAUAUAGGA |
| SEQ ID NO: 6 | FluA_cr06m | gaccaccccaaaaaugaaggggacuaaaacGUGCCAUAGAG |
| | | GAGACACAC |
| SEQ ID NO: 7 | FluA_cr07 | gaccaccccaaaaaugaaggggacuaaaacUAUGGCCAUAA |
| | | UUAAGAAGU |
| SEQ ID NO: 8 | FluA_cr08 | gaccaccccaaaaaugaaggggacuaaaacAAUUUUUCCCU |
| | | AGUAGUUCA |
| SEQ ID NO: 9 | FluA_cr09 | gaccaccccaaaaaugaaggggacuaaaacUCAGCUGACAU |
| | | GAGUAUUGG |
| SEQ ID NO: 10 | FluA_cr10 | gaccaccccaaaaaugaaggggacuaaaacCAGCUGACAUG |
| | | AGUAUUGGA |
| SEQ ID NO: 11 | FluA_cr11 | gaccaccccaaaaaugaaggggacuaaaacGACAUGAGUAU |
| | | UGGAGUAAC |
| SEQ ID NO: 12 | FluA_cr12 | gaccaccccaaaaaugaaggggacuaaaacCCUGAAGUCUG |
| | | CUUAAAGUG |
| SEQ ID NO: 13 | FluA_cr13 | gaccaccccaaaaaugaaggggacuaaaacGAGUCAGGAAG |
| | | GCUAAUAGA |
| SEQ ID NO: 14 | FluA_cr14 | gaccaccccaaaaaugaaggggacuaaaacCGGUUGGAAUU |
| | | UCUAGCAUG |
| SEQ ID NO: 15 | FluA_cr15 | gaccaccccaaaaaugaaggggacuaaaacGGUUGGAAUUU |
| | | CUAGCAUGG |
| SEQ ID NO: 16 | FluA_cr16 | gaccaccccaaaaaugaaggggacuaaaacAUAUGAAGCAA |
| | | UCGAGGAGU |
| SEQ ID NO: 17 | FluA_cr17 | gaccaccccaaaaaugaaggggacuaaaacUAUGAAGCAAU |
| | | CGAGGAGUG |
| SEQ ID NO: 18 | FluA_cr18 | gaccaccccaaaaaugaaggggacuaaaacCCUGCCUGCUU |
| | | GUGUGUAUG |
| SEQ ID NO: 19 | FluA_cr19 | gaccaccccaaaaaugaaggggacuaaaacCGGCAAUGGUG |
| | | UUUGGAUAG |
| SEQ ID NO: 20 | FluA_cr20 | gaccaccccaaaaaugaaggggacuaaaacAACGUACGUUC |
| | | UUUCUAUCA |
| SEQ ID NO: 21 | FluA_cr21 | gaccaccccaaaaaugaaggggacuaaaacUAUACAGAGAU |
| | | UCGCUUGGA |
| SEQ ID NO: 22 | FluA_cr22 | gaccaccccaaaaaugaaggggacuaaaacAUACAGAGAUU |
| | | CGCUUGGAG |
| SEQ ID NO: 23 | FluB_cr01 | gaccaccccaaaaaugaaggggacuaaaacUACUACAAAAA |
| | | UCCCGGAAC |
| SEQ ID NO: 24 | FluB_cr02 | gaccaccccaaaaaugaaggggacuaaaacAGAUAGCAUAU |
| | | UAAACAUUC |
| SEQ ID NO: 25 | FluB_cr03 | gaccaccccaaaaaugaaggggacuaaaacAUUUAUCCCAU |
| | | GUAGCUCAC |
| SEQ ID NO: 26 | FluB_cr04 | gaccaccccaaaaaugaaggggacuaaaacGACAUCAUUCU |
| | | GCCGCAUAU |
| SEQ ID NO: 27 | FluB_cr05 | gaccaccccaaaaaugaaggggacuaaaacAGACAUCUUCU |
| | | AGCUUCCAU |
| SEQ ID NO: 28 | FluB_cr06 | gaccaccccaaaaaugaaggggacuaaaacUCCCAGUGCAG |
| | | AUUCGAUCU |
| SEQ ID NO: 29 | FluB_cr07 | gaccaccccaaaaaugaaggggacuaaaacAUCCCAGUGCA |
| | | GAUUCGAUC |
| SEQ ID NO: 30 | FluB_cr08 | gaccaccccaaaaaugaaggggacuaaaacUAUCCCAGUGC |
| | | AGAUUCGAU |
| SEQ ID NO: 31 | FluB_cr09 | gaccaccccaaaaaugaaggggacuaaaacUUAUCCCAGUG |
| | | CAGAUUCGA |
| SEQ ID NO: 32 | FluB_cr10 | gaccaccccaaaaaugaaggggacuaaaacGUCGUGCAUUA |
| | | UAGGAAAGC |
| SEQ ID NO: 33 | FluB_cr11 | gaccaccccaaaaaugaaggggacuaaaacUUCAUACCCAA |
| | | CCAUAGAGU |
| SEQ ID NO: 34 | FluB_cr12 | gaccaccccaaaaaugaaggggacuaaaacGACAGCAUUCU |
| | | UCUUACAGC |
| SEQ ID NO: 35 | FluB_cr13 | gaccaccccaaaaaugaaggggacuaaaacGAUAAGACUCC |
| | | CACCGCAGU |
| SEQ ID NO: 36 | FluB_cr14 | gaccaccccaaaaaugaaggggacuaaaacGCUGUACACUU |
| | | CAACCACAU |
| SEQ ID NO: 37 | FluB_cr15 | gaccaccccaaaaaugaaggggacuaaaacUGCCUGCUGUA |
| | | CACUUCAAC |

As illustrated herein, for detection of Influenza B, crRNAs with a sequence of SEQ ID NOs: 32, 34, 35, 36 exhibit better signals than crRNAs with a sequence of SEQ ID NOs: 23-31, 33, or 37. Moreover, the combination of the crRNAs of SEQ ID NOs: 34 and 36 significantly improves detection of Influenza B over using crRNAs of SEQ ID NOs: 34 or 36 alone.

To detect Influenza A, crRNAs with a sequence of SEQ ID NOs: 4, 8, 13, 16, 17, 21, or 22 exhibit better signals than crRNAs with a sequence of SEQ ID NOs: 1-3, 5-7, 9-12, 14, 15, or 18-20. Moreover, the combination of the seven crRNAs of SEQ ID NOs: 4, 8, 13, 16, 17, 21, and 22 and independently the combination of the four crRNAs of SEQ ID NOs: 8, 16, 21, or 22 significantly improves detection of Influenza A over using the crRNAs of SEQ ID NOs: 4, 8, 13, 16, 17, 21, or 22 alone.

### Influenza Sequences

A DNA sequence for the Influenza A genome, strain H1N1, with coding regions for each of the eight single stranded RNA segments, is available under the following accession numbers:

```
Segment 1: NC_002023.1 from the NCBI website (provided as SEQ ID NO: 51 herein).
      1 AGCGAAAGCA GGTCAATTAT ATTCAATATG GAAAGAATAA AAGAACTAAG AAATCTAATG
    61 TCGCAGTCTC GCACCCGCGA GATACTCACA AAAACCACCG TGGACCATAT GGCCATAATC
  121 AAGAAGTACA CATCAGGAAG ACAGGAGAAG AACCCAGCAC TTAGGATGAA ATGGATGATG
  181 GCAATGAAAT ATCCAATTAC AGCAGACAAG AGGATAACGG AAATGATTCC TGAGAGAAAT
  241 GAGCAAGGAC AAACTTTATG GAGTAAAATG AATGATGCCG GATCAGACCG AGTGATGGTA
  301 TCACCTCTGG CTGTGACATG GTGGAATAGG AATGGACCAA TGACAAATAC AGTTCATTAT
  361 CCAAAAATCT ACAAAACTTA TTTTGAAAGA GTCGAAAGGC TAAAGCATGG AACCTTTGGC
  421 CCTGTCCATT TTAGAAACCA AGTCAAAATA CGTCGGAGAG TTGACATAAA TCCTGGTCAT
  481 GCAGATCTCA GTGCCAAGGA GGCACAGGAT GTAATCATGG AAGTTGTTTT CCCTAACGAA
  541 GTGGGAGCCA GGATACTAAC ATCGGAATCG CAACTAACGA TAACCAAAGA GAAGAAAGAA
  601 GAACTCCAGG ATTGCAAAAT TTCTCCTTTG ATGGTTGCAT ACATGTTGGA GAGAGAACTG
  661 GTCCGCAAAA CGAGATTCCT CCCAGTGGCT GGTGGAACAA GCAGTGTGTA CATTGAAGTG
  721 TTGCATTTGA CTCAAGGAAC ATGCTGGGAA CAGATGTATA CTCCAGGAGG GGAAGTGAAG
  781 AATGATGATG TTGATCAAAG CTTGATTATT GCTGCTAGGA ACATAGTGAG AAGAGCTGCA
  841 GTATCAGCAG ACCCACTAGC ATCTTTATTG GAGATGTGCC ACAGCACACA GATTGGTGGA
  901 ATTAGGATGG TAGACATCCT TAAGCAGAAC CCAACAGAAG AGCAAGCCGT GGGTATATGC
  961 AAGGCTGCAA TGGGACTGAG AATTAGCTCA TCCTTCAGTT TTGGTGGATT CACATTTAAG
1021 AGAACAAGCG GATCATCAGT CAAGAGAGAG GAAGAGGTGC TTACGGGCAA TCTTCAAACA
1081 TTGAAGATAA GAGTGCATGA GGGATATGAA GAGTTCACAA TGGTTGGGAG AAGAGCAACA
1141 GCCATACTCA GAAAAGCAAC CAGGAGATTG ATTCAGCTGA TAGTGAGTGG GAGAGACGAA
1201 CAGTCGATTG CCGAAGCAAT AATTGTGGCC ATGGTATTTT CACAAGAGGA TTGTATGATA
1261 AAAGCAGTTA GAGGTGATCT GAATTTCGTC AATAGGGCGA ATCAGCGACT GAATCCTATG
1321 CATCAACTTT TAAGACATTT TCAGAAGGAT GCGAAAGTGC TTTTTCAAAA TTGGGGAGTT
1381 GAACCTATCG ACAATGTGAT GGGAATGATT GGGATATTGC CCGACATGAC TCCAAGCATC
1441 GAGATGTCAA TGAGAGGAGT GAGAATCAGC AAAATGGGTG TAGATGAGTA CTCCAGCACG
1501 GAGAGGGTAG TGGTGAGCAT TGACCGGTTC TTGAGAGTCC GGGACCAACG AGGAAATGTA
1561 CTACTGTCTC CCGAGGAGGT CAGTGAAACA CAGGGAACAG AGAAACTGAC AATAACTTAC
1621 TCATCGTCAA TGATGTGGGA GATTAATGGT CCTGAATCAG TGTTGGTCAA TACCTATCAA
1681 TGGATCATCA GAAACTGGGA AACTGTTAAA ATTCAGTGGT CCCAGAACCC TACAATGCTA
1741 TACAATAAAA TGGAATTTGA ACCATTTCAG TCTTTAGTAC CTAAGGCCAT TAGAGGCCAA
1801 TACAGTGGGT TTGTGAGAAC TCTGTTCCAA CAAATGAGGG ATGTGCTTGG GACATTTGAT
1861 ACCGCACAGA TAATAAAACT TCTTCCCTTC GCAGCCGCTC CACCAAAGCA AAGTAGAATG
1921 CAGTTCTCCT CATTTACTGT GAATGTGAGG GGATCAGGAA TGAGAATACT TGTAAGGGGC
1981 AATTCTCCTG TATTCAACTA CAACAAGGCC ACGAAGAGAC TCACAGTTCT CGGAAAGGAT
2041 GCTGGCACTT TAACCGAAGA CCCAGATGAA GGCACAGCTG GAGTGGAGTC CGCTGTTCTG
2101 AGGGGATTCC TCATTCTGGG CAAAGAAGAC AGGAGATATG GGCCAGCATT AAGCATCAAT
2161 GAACTGAGCA ACCTTGCGAA AGGAGAGAAG GCTAATGTGC TAATTGGGCA AGGAGACGTG
2221 GTGTTGGTAA TGAAACGAAA ACGGGACTCT AGCATACTTA CTGACAGCCA GACAGCGACC
2281 AAAAGAATTC GGATGGCCAT CAATTAGTGT CGAATAGTTT AAAAACGACC TTGTTTCTAC
2341 T
Segment 2: NC_002021.1 from the NCBI website (provided as SEQ ID NO: 52 herein).
      1 AGCGAAAGCA GGCAAACCAT TTGAATGGAT GTCAATCCGA CCTTACTTTT CTTAAAAGTG
    61 CCAGCACAAA ATGCTATAAG CACAACTTTC CCTTATACCG GAGACCCTCC TTACAGCCAT
  121 GGGACAGGAA CAGGATACAC CATGGATACT GTCAACAGGA CACATCAGTA CTCAGAAAAG
  181 GCAAGATGGA CAACAAACAC CGAAACTGGA GCACCGCAAC TCAACCCGAT TGATGGGCCA
  241 CTGCCAGAAG ACAATGAACC AAGTGGTTAT GCCCAAACAG ATTGTGTATT GGAAGCAATG
  301 GCTTTCCTTG AGGAATCCCA TCCTGGTATT TTTGAAAACT CGTGTATTGA AACGATGGAG
  361 GTTGTTCAGC AAACACGAGT AGACAAGCTG ACACAAGGCC GACAGACCTA TGACTGGACT
  421 TTAAATAGAA ACCAGCCTGC TGCAACAGCA TTGGCCAACA CAATAGAAGT GTTCAGATCA
  481 AATGGCCTCA CGGCCAATGA GTCTGGAAGG CTCATAGACT TCCTTAAGGA TGTAATGGAG
  541 TCAATGAAAA AAGAAGAAAT GGGGATCACA ACTCATTTTC AGAGAAAGAG ACGGGTGAGA
  601 GACAATATGA CTAAGAAAAT GATAACACAG AGAACAATAG GTAAAAGGAA ACAGAGATTG
  661 AACAAAAGGA GTTATCTAAT TAGAGCATTG ACCCTGAACA CAATGACCAA AGATGCTGAG
  721 AGAGGGAAGC TAAAACGGAG AGCAATTGCA ACCCCAGGGA TGCAAATAAG GGGGTTTGTA
  781 TACTTTGTTG AGACACTGGC AAGGAGTATA TGTGAGAAAC TTGAACAATC AGGGTTGCCA
  841 GTTGGAGGCA ATGAGAAGAA AGCAAAGTTG GCAAATGTTG TAAGGAAGAT GATGACCAAT
  901 TCTCAGGACA CCGAACTTTC TTTGACCATC ACTGGAGATA ACACCAAATG GAACGAAAAT
  961 CAGAATCCTC GGATGTTTTT GGCCATGATC ACATATATGA CCAGAAATCA GCCCGAATGG
1021 TTCAGAAATG TTCTAAGTAT TGCTCCAATA ATGTTCTCAA ACAAAATGGC GAGACTGGGA
1081 AAAGGGTATA TGTTTGAGAG CAAGAGTATG AAACTTAGAA CTCAAATACC TGCAGAAATG
1141 CTAGCAAGCA TTGATTTGAA ATATTTCAAT GATTCAACAA GAAAGAAGAT TGAAAAAATC
1201 CGACCGCTCT TAATAGAGGG GACTGCATCA TTGAGCCCTG GAATGATGAT GGGCATGTTC
1261 AATATGTTAA GCACTGTATT AGGCGTCTCC ATCCTGAATC TTGGACAAAA GAGATACACC
1321 AAGACTACTT ACTGGTGGGA TGGTCTTCAA TCCTCTGACG ATTTTGCTCT GATTGTGAAT
1381 GCACCCAATC ATGAAGGGAT TCAAGCCGGA GTCGACAGGT TTTATCGAAC CTGTAAGCTA
1441 CATGGAATCA ATATGAGCAA GAAAAAGTCT TACATAAACA GAACAGGTAC ATTTGAATTC
1501 ACAAGTTTTT TCTATCGTTA TGGGTTTGTT GCCAATTTCA GCATGGAGCT TCCCAGTTTT
1561 GGTGTGTCTG GGAGCAACGA GTCAGCGGAC ATGAGTATTG GAGTTACTGT CATCAAAAAC
1621 AATATGATAA ACAATGATCT TGGTCCAGCA ACAGCTCAAA TGGCCCTTCA GTTGTTCATC
1681 AAAGATTACA GGTACACGTA CCGATGCCAT AGAGGTGACA CACAAATACA AACCCGAAGA
1741 TCATTTGAAA TAAAGAAACT GTGGGAGCAA ACCCGTTCCA AAGCTGGACT GCTGGTCTCC
1801 GACGGAGGCC CAAATTTATA CAACATTAGA AATCTCCACA TTCCTGAAGT CTGCCTAAAA
1861 TGGGAATTGA TGGATGAGGA TTACCAGGGG CGTTTATGCA ACCCACTGAA CCCATTTGTC
1921 AGCCATAAAG AAATTGAATC AATGAACAAT GCAGTGATGA TGCCAGCACA TGGTCCAGCC
1981 AAAAACATGG AGTATGATGC TGTTGCAACA ACACACTCCT GGATCCCCAA AAGAAATCGA
2041 TCCATCTTGA ATACAAGTCA AAGAGGAGTA CTTGAAGATG AACAAATGTA CCAAAGGTGC
2101 TGCAATTTAT TTGAAAAATT CTTCCCCAGC AGTTCATACA GAAGACCAGT CGGGATATCC
2161 AGTATGGTGG AGGCTATGGT TTCCAGAGCC CGAATTGATG CACGGATTGA TTTCGAATCT
2221 GGAAGGATAA AGAAAGAAGA GTTCACTGAG ATCATGAAGA TCTGTTCCAC CATTGAAGAG
2281 CTCAGACGGC AAAAATAGTG AATTTAGCTT GTCCTTCATG AAAAAATGCC TTGTTCCTAC
2341 T
Segment 3: NC_002022.1 from the NCBI website (provided as SEQ ID NO: 53 herein).
      1 AGCGAAAGCA GGTACTGATC CAAAATGGAA GATTTTGTGC GACAATGCTT CAATCCGATG
    61 ATTGTCGAGC TTGCGGAAAA AACAATGAAA GAGTATGGGG AGGACCTGAA AATCGAAACA
  121 AACAAATTTG CAGCAATATG CACTCACTTG GAAGTATGCT TCATGTATTC AGATTTCCAC
  181 TTCATCAATG AGCAAGGCGA GTCAATAATC GTAGAACTTG GTGATCCTAA TGCACTTTTG
  241 AAGCACAGAT TTGAAATAAT CGAGGGAAGA GATCGCACAA TGGCCTGGAC AGTAGTAAAC
  301 AGTATTTGCA ACACTACAGG GGCTGAGAAA CCAAAGTTTC TACCAGATTT GTATGATTAC
  361 AAGGAAAATA GATTCATCGA AATTGGAGTA ACAAGGAGAG AAGTTCACAT ATACTATCTG
  421 GAAAAGGCCA ATAAAATTAA ATCTGAGAAA ACACACATCC ACATTTTCTC GTTCACTGGG
  481 GAAGAAATGG CCACAAAGGC CGACTACACT CTCGATGAAG AAAGCAGGGC TAGGATCAAA
  541 ACCAGGCTAT TCACCATAAG ACAAGAAATG GCCAGCAGAG GCCTCTGGGA TTCCTTTCGT
  601 CAGTCCGAGA GAGGAGAAGA GACAATTGAA GAAAGGTTTG AAATCACAGG AACAATGCGC
  661 AAGCTTGCCG ACCAAAGTCT CCCGCCGAAC TTCTCCAGCC TTGAAAATTT TAGAGCCTAT
  721 GTGGATGGAT TCGAACCGAA CGGCTACATT GAGGGCAAGC TGTCTCAAAT GTCCAAAGAA
  781 GTAAATGCTA GAATTGAACC TTTTTTGAAA ACAACACCAC GACCACTTAG ACTTCCGAAT
  841 GGGCCTCCCT GTTCTCAGCG GTCCAAATTC CTGCTGATGG ATGCCTTAAA ATTAAGCATT
  901 GAGGACCCAA GTCATGAAGG AGAGGGAATA CCGCTATATG ATGCAATCAA ATGCATGAGA
  961 ACATTCTTTG GATGGAAGGA ACCCAATGTT GTTAAACCAC ACGAAAAGGG AATAAATCCA
1021 AATTATCTTC TGTCATGGAA GCAAGTACTG GCAGAACTGC AGGACATTGA GAATGAGGAG
1081 AAAATTCCAA AGACTAAAAA TATGAAAAAA ACAAGTCAGC TAAAGTGGGC ACTTGGTGAG
1141 AACATGGCAC CAGAAAAGGT AGACTTTGAC GACTGTAAAG ATGTAGGTGA TTTGAAGCAA
1201 TATGATAGTG ATGAACCAGA ATTGAGGTCG CTTGCAAGTT GGATTCAGAA TGAGTTCAAC
1261 AAGGCATGCG AACTGACAGA TTCAAGCTGG ATAGAGCTTG ATGAGATTGG AGAAGATGTG
1321 GCTCCAATTG AACACATTGC AAGCATGAGA AGGAATTATT TCACATCAGA GGTGTCTCAC
1381 TGCAGAGCCA CAGAATACAT AATGAAGGGG GTGTACATCA ATACTGCCTT ACTTAATGCA
1441 TCTTGTGCAG CAATGGATGA TTTCCAATTA ATTCCAATGA TAAGCAAGTG TAGAACTAAG
1501 GAGGGAAGGC GAAAGACCAA CTTGTATGGT TTCATCATAA AAGGAAGATC CCACTTAAGG
1561 AATGACACCG ACGTGGTAAA CTTTGTGAGC ATGGAGTTTT CTCTCACTGA CCCAAGACTT
1621 GAACCACACA AATGGGAGAA GTACTGTGTT CTTGAGATAG GAGATATGCT TCTAAGAAGT
1681 GCCATAGGCC AGGTTTCAAG GCCCATGTTC TTGTATGTGA GGACAAATGG AACCTCAAAA
1741 ATTAAAATGA AATGGGGAAT GGAGATGAGG CGTTGTCTCC TCCAGTCACT TCAACAAATT
1801 GAGAGTATGA TTGAAGCTGA GTCCTCTGTC AAAGAGAAAG ACATGACCAA AGAGTTCTTT
1861 GAGAACAAAT CAGAAACATG GCCCATTGGA GAGTCTCCCA AAGGAGTGGA GGAAAGTTCC
1921 ATTGGGAAGG TCTGCAGGAC TTTATTAGCA AAGTCGGTAT TTAACAGCTT GTATGCATCT
1981 CCACAACTAG AAGGATTTTC AGCTGAATCA AGAAAACTGC TTCTTATCGT TCAGGCTCTT
2041 AGGGACAATC TGGAACCTGG GACCTTTGAT CTTGGGGGGC TATATGAAGC AATTGAGGAG
2101 TGCCTAATTA ATGATCCCTG GGTTTTGCTT AATGCTTCTT GGTTCAACTC CTTCCTTACA
2161 CATGCATTGA GTTAGTTGTG GCAGTGCTAC TATTTGCTAT CCATACTGTC CAAAAAAGTA
2221 CCTTGTTTCT ACT
Segment 4: NC_002017.1 from the NCBI website (provided as SEQ ID NO: 54 herein).
      1 AGCAAAAGCA GGGGAAAATA AAAACAACCA AAATGAAGGC AAACCTACTG GTCCTGTTAT
    61 GTGCACTTGC AGCTGCAGAT GCAGACACAA TATGTATAGG CTACCATGCG AACAATTCAA
  121 CCGACACTGT TGACACAGTG CTCGAGAAGA ATGTGACAGT GACACACTCT GTTAACCTGC
  181 TCGAAGACAG CCACAACGGA AAACTATGTA GATTAAAAGG AATAGCCCCA CTACAATTGG
  241 GGAAATGTAA CATCGCCGGA TGGCTCTTGG GAAACCCAGA ATGCGACCCA CTGCTTCCAG
  301 TGAGATCATG GTCCTACATT GTAGAAACAC CAAACTCTGA GAATGGAATA TGTTATCCAG
  361 GAGATTTCAT CGACTATGAG GAGCTGAGGG AGCAATTGAG CTCAGTGTCA TCATTCGAAA
  421 GATTCGAAAT ATTTCCCAAA GAAAGCTCAT GGCCCAACCA CAACACAACC AAAGGAGTAA
  481 CGGCAGCATG CTCCCATGCG GGGAAAAGCA GTTTTTACAG AAATTTGCTA TGGCTGACGG
  541 AGAAGGAGGG CTCATACCCA AAGCTGAAAA ATTCTTATGT GAACAAGAAA GGGAAAGAAG
  601 TCCTTGTACT GTGGGGTATT CATCACCCGT CTAACAGTAA GGATCAACAG AATATCTATC
  661 AGAATGAAAA TGCTTATGTC TCTGTAGTGA CTTCAAATTA TAACAGGAGA TTTACCCCGG
  721 AAATAGCAGA AAGACCCAAA GTAAGAGATC AAGCTGGGAG GATGAACTAT TACTGGACCT
  781 TGCTAAAACC CGGAGACACA ATAATATTTG AGGCAAATGG AAATCTAATA GCACCAAGGT
  841 ATGCTTTCGC ACTGAGTAGA GGCTTTGGGT CCGGCATCAT CACCTCAAAC GCATCAATGC
  901 ATGAGTGTAA CACGAAGTGT CAAACACCCC TGGGAGCTAT AAACAGCAGT CTCCCTTTCC
  961 AGAATATACA CCCAGTCACA ATAGGAGAGT GCCCAAAATA CGTCAGGAGT GCCAAATTGA
1021 GGATGGTTAC AGGACTAAGG AACATTCCGT CCATTCAATC CAGAGGTCTA TTTGGAGCCA
1081 TTGCCGGTTT TATTGAAGGG GGATGGACTG GAATGATAGA TGGATGGTAC GGTTATCATC
1141 ATCAGAATGA ACAGGGATCA GGCTATGCAG CGGATCAAAA AAGCACACAA AATGCCATTA
1201 ACGGGATTAC AAACAAGGTG AACTCTGTTA TCGAGAAAAT GAACATTCAA TTCACAGCTG
1261 TGGGTAAAGA ATTCAACAAA TTAGAAAAAA GGATGGAAAA TTTAAATAAA AAAGTTGATG
1321 ATGGATTTCT GGACATTTGG ACATATAATG CAGAATTGTT AGTTCTACTG GAAAATGAAA
1381 GGACTCTGGA TTTCCATGAC TCAAATGTGA AGAATCTGTA TGAGAAAGTA AAAAGCCAAT
1441 TAAAGAATAA TGCCAAAGAA ATCGGAAATG GATGTTTTGA GTTCTACCAC AAGTGTGACA
1501 ATGAATGCAT GGAAAGTGTA AGAAATGGGA CTTATGATTA TCCCAAATAT TCAGAAGAGT
1561 CAAAGTTGAA CAGGGAAAAG GTAGATGGAG TGAAATTGGA ATCAATGGGG ATCTATCAGA
1621 TTCTGGCGAT CTACTCAACT GTCGCCAGTT CACTGGTGCT TTTGGTCTCC CTGGGGGCAA
1681 TCAGTTTCTG GATGTGTTCT AATGGATCTT TGCAGTGCAG AA
Segment 5: NC_002019.1 from the NCBI website (provided as SEQ ID NO: 55 herein).
      1 AGCAAAAGCA GGGTAGATAA TCACTCACTG AGTGACATCA AAATCATGGC GTCCCAAGGC
    61 ACCAAACGGT CTTACGAACA GATGGAGACT GATGGAGAAC GCCAGAATGC CACTGAAATC
  121 AGAGCATCCG TCGGAAAAAT GATTGGTGGA ATTGGACGAT TCTACATCCA AATGTGCACA
  181 GAACTTAAAC TCAGTGATTA TGAGGGACGG TTGATCCAAA ACAGCTTAAC AATAGAGAGA
  241 ATGGTGCTCT CTGCTTTTGA CGAAAGGAGA AATAAATACC TGGAAGAACA TCCCAGTGCG
  301 GGGAAGGATC CTAAGAAAAC TGGAGGACCT ATATACAGAA GAGTAAACGG AAAGTGGATG
  361 AGAGAACTCA TCCTTTATGA CAAAGAAGAA ATAAGGCGAA TCTGGCGCCA AGCTAATAAT
  421 GGTGACGATG CAACGGCTGG TCTGACTCAC ATGATGATCT GGCATTCCAA TTTGAATGAT
  481 GCAACTTATC AGAGGACAAG GGCTCTTGTT CGCACCGGAA TGGATCCCAG GATGTGCTCT
  541 CTGATGCAAG GTTCAACTCT CCCTAGGAGG TCTGGAGCCG CAGGTGCTGC AGTCAAAGGA
  601 GTTGGAACAA TGGTGATGGA ATTGGTCAGG ATGATCAAAC GTGGGATCAA TGATCGGAAC
  661 TTCTGGAGGG GTGAGAATGG ACGAAAAACA AGAATTGCTT ATGAAAGAAT GTGCAACATT
  721 CTCAAAGGGA AATTTCAAAC TGCTGCACAA AAAGCAATGA TGGATCAAGT GAGAGAGAGC
  781 CGGGACCCAG GGAATGCTGA GTTCGAAGAT CTCACTTTTC TAGCACGGTC TGCACTCATA
  841 TTGAGAGGGT CGGTTGCTCA CAAGTCCTGC CTGCCTGCCT GTGTGTATGG ACCTGCCGTA
  901 GCCAGTGGGT ACGACTTTGA AAGAGAGGGA TACTCTCTAG TCGGAATAGA CCCTTTCAGA
  961 CTGCTTCAAA ACAGCCAAGT GTACAGCCTA ATCAGACCAA ATGAGAATCC AGCACACAAG
1021 AGTCAACTGG TGTGGATGGC ATGCCATTCT GCCGCATTTG AAGATCTAAG AGTATTGAGC
1081 TTCATCAAAG GGACGAAGGT GGTCCCAAGA GGGAAGCTTT CCACTAGAGG AGTTCAAATT
1141 GCTTCCAATG AAAATATGGA GACTATGGAA TCAAGTACAC TTGAACTGAG AAGCAGGTAC
1201 TGGGCCATAA GGACCAGAAG TGGAGGAAAC ACCAATCAAC AGAGGGCATC TGCGGGCCAA
1261 ATCAGCATAC AACCTACGTT CTCAGTACAG AGAAATCTCC CTTTTGACAG AACAACCGTT
1321 ATGGCAGCAT TCACTGGGAA TACAGAGGGG AGAACATCTG ACATGAGGAC CGAAATCATA
1381 AGGATGATGG AAAGTGCAAG ACCAGAAGAT GTGTCTTTCC AGGGGCGGGG AGTCTTCGAG
1441 CTCTCGGACG AAAAGGCAGC GAGCCCGATC GTGCCTTCCT TTGACATGAG TAATGAAGGA
1501 TCTTATTTCT TCGGAGACAA TGCAGAGGAG TACGACAATT AAAGAAAAAT ACCCTTGTTT
1561 CTACT
Segment 6: NC_002018.1 from the NCBI website (provided as SEQ ID NO: 56 herein).
      1 AGCGAAAGCA GGGGTTTAAA ATGAATCCAA ATCAGAAAAT AATAACCATT GGATCAATCT
    61 GTCTGGTAGT CGGACTAATT AGCCTAATAT TGCAAATAGG GAATATAATC TCAATATGGA
  121 TTAGCCATTC AATTCAAACT GGAAGTCAAA ACCATACTGG AATATGCAAC CAAAACATCA
  181 TTACCTATAA AAATAGCACC TGGGTAAAGG ACACAACTTC AGTGATATTA ACCGGCAATT
  241 CATCTCTTTG TCCCATCCGT GGGTGGGCTA TATACAGCAA AGACAATAGC ATAAGAATTG
  301 GTTCCAAAGG AGACGTTTTT GTCATAAGAG AGCCCTTTAT TTCATGTTCT CACTTGGAAT
  361 GCAGGACCTT TTTTCTGACC CAAGGTGCCT TACTGAATGA CAGGCATTCA AATGGGACTG
  421 TTAAGGACAG AAGCCCTTAT AGGGCCTTAA TGAGCTGCCC TGTCGGTGAA GCTCCGTCCC
  481 CGTACAATTC AAGATTTGAA TCGGTTGCTT GGTCAGCAAG TGCATGTCAT GATGGCATGG
  541 GCTGGCTAAC AATCGGAATT TCAGGTCCAG ATAATGGAGC AGTGGCTGTA TTAAAATACA
  601 ACGGCATAAT AACTGAAACC ATAAAAAGTT GGAGGAAGAA AATATTGAGG ACACAAGAGT
  661 CTGAATGTGC CTGTGTAAAT GGTTCATGTT TTACTATAAT GACTGATGGC CCGAGTGATG
  721 GGCTGGCCTC GTACAAAATT TTCAAGATCG AAAAGGGGAA GGTTACTAAA TCAATAGAGT
  781 TGAATGCACC TAATTCTCAC TATGAGGAAT GTTCCTGTTA CCCTGATACC GGCAAAGTGA
  841 TGTGTGTGTG CAGAGACAAT TGGCATGGTT CGAACCGGCC ATGGGTGTCT TTCGATCAAA
  901 ACCTGGATTA TCAAATAGGA TACATCTGCA GTGGGGTTTT CGGTGACAAC CCGCGTCCCA
  961 AAGATGGAAC AGGCAGCTGT GGTCCAGTGT ATGTTGATGG AGCAAACGGA GTAAAGGGAT
1021 TTTCATATAG GTATGGTAAT GGTGTTTGGA TAGGAAGGAC CAAAAGTCAC AGTTCCAGAC
1081 ATGGGTTTGA GATGATTTGG GATCCTAATG GATGGACAGA GACTGATAGT AAGTTCTCTG
1141 TGAGGCAAGA TGTTGTGGCA ATGACTGATT GGTCAGGGTA TAGCGGGAGT TTCGTTCAAC
1201 ATCCTGAGCT AACAGGGCTA GACTGTATAA GGCCGTGCTT CTGGGTTGAA TTAATCAGGG
1261 GACGACCTAA AGAAAAAACA ATCTGGACTA GTGCGAGCAG CATTTCTTTT TGTGGCGTGA
1321 ATAGTGATAC TGTAGATTGG TCTTGGCCAG ACGGTGCTGA GTTGCCATTC ACCATTGACA
1381 AGTAGTCTGT TCAAAAAACT CCTTGTTTCT ACT
Segment 7: NC_002016.1 from the NCBI website (provided as SEQ ID NO: 57 herein).
      1 AGCGAAAGCA GGTAGATATT GAAAGATGAG TCTTCTAACC GAGGTCGAAA CGTACGTTCT
    61 CTCTATCATC CCGTCAGGCC CCCTCAAAGC CGAGATCGCA CAGAGACTTG AAGATGTCTT
  121 TGCAGGGAAG AACACCGATC TTGAGGTTCT CATGGAATGG CTAAAGACAA GACCAATCCT
  181 GTCACCTCTG ACTAAGGGGA TTTTAGGATT TGTGTTCACG CTCACCGTGC CCAGTGAGCG
  241 AGGACTGCAG CGTAGACGCT TTGTCCAAAA TGCCCTTAAT GGGAACGGGG ATCCAAATAA
  301 CATGGACAAA GCAGTTAAAC TGTATAGGAA GCTCAAGAGG GAGATAACAT TCCATGGGGC
  361 CAAAGAAATC TCACTCAGTT ATTCTGCTGG TGCACTTGCC AGTTGTATGG GCCTCATATA
  421 CAACAGGATG GGGGCTGTGA CCACTGAAGT GGCATTTGGC CTGGTATGTG CAACCTGTGA
  481 ACAGATTGCT GACTCCCAGC ATCGGTCTCA TAGGCAAATG GTGACAACAA CCAACCCACT
  541 AATCAGACAT GAGAACAGAA TGGTTTTAGC CAGCACTACA GCTAAGGCTA TGGAGCAAAT
  601 GGCTGGATCG AGTGAGCAAG CAGCAGAGGC CATGGAGGTT GCTAGTCAGG CTAGGCAAAT
  661 GGTGCAAGCG ATGAGAACCA TTGGGACTCA TCCTAGCTCC AGTGCTGGTC TGAAAAATGA
  721 TCTTCTTGAA AATTTGCAGG CCTATCAGAA ACGAATGGGG GTGCAGATGC AACGGTTCAA
  781 GTGATCCTCT CGCTATTGCC GCAAATATCA TTGGGATCTT GCACTTGATA TTGTGGATTC
  841 TTGATCGTCT TTTTTTCAAA TGCATTTACC GTCGCTTTAA ATACGGACTG AAAGGAGGGC
  901 CTTCTACGGA AGGAGTGCCA AAGTCTATGA GGGAAGAATA TCGAAAGGAA CAGCAGAGTG
  961 CTGTGGATGC TGACGATGGT CATTTTGTCA GCATAGAGCT GGAGTAAAAA ACTACCTTGT
1021 TTCTACT
Segment 8: NC_002020.1 from the NCBI website (provided as SEQ ID NO: 58 herein).
      1 AGCAAAAGCA GGGTGACAAA GACATAATGG ATCCAAACAC TGTGTCAAGC TTTCAGGTAG
    61 ATTGCTTTCT TTGGCATGTC CGCAAACGAG TTGCAGACCA AGAACTAGGT GATGCCCCAT
  121 TCCTTGATCG GCTTCGCCGA GATCAGAAAT CCCTAAGAGG AAGGGGCAGC ACTCTTGGTC
  181 TGGACATCGA GACAGCCACA CGTGCTGGAA AGCAGATAGT GGAGCGGATT CTGAAAGAAG
  241 AATCCGATGA GGCACTTAAA ATGACCATGG CCTCTGTACC TGCGTCGCGT TACCTAACCG
  301 ACATGACTCT TGAGGAAATG TCAAGGGAAT GGTCCATGCT CATACCCAAG CAGAAAGTGG
  361 CAGGCCCTCT TTGTATCAGA ATGGACCAGG CGATCATGGA TAAAAACATC ATACTGAAAG
  421 CGAACTTCAG TGTGATTTTT GACCGGCTGG AGACTCTAAT ATTGCTAAGG GCTTTCACCG
  481 AAGAGGGAGC AATTGTTGGC GAAATTTCAC CATTGCCTTC TCTTCCAGGA CATACTGCTG
  541 AGGATGTCAA AAATGCAGTT GGAGTCCTCA TCGGAGGACT TGAATGGAAT GATAACACAG
  601 TTCGAGTCTC TGAAACTCTA CAGAGATTCG CTTGGAGAAG CAGTAATGAG AATGGGAGAC
  661 CTCCACTCAC TCCAAAACAG AAACGAGAAA TGGCGGGAAC AATTAGGTCA GAAGTTTGAA
  721 GAAATAAGAT GGTTGATTGA AGAAGTGAGA CACAAACTGA AGGTAACAGA GAATAGTTTT
  781 GAGCAAATAA CATTTATGCA AGCCTTACAT CTATTGCTTG AAGTGGAGCA AGAGATAAGA
841 ACTTTCTCAT TTCAGCTTAT TTAATAATAA AAAACACCCT TGTTTCTACT
```

A DNA sequence for the Influenza B genome, strain Bisbane, with coding regions for each of the eight single stranded RNA segments, is available under the following accession numbers:

```
Segment 1: CY018707.1 from the NCBI website (provided as SEQ ID NO: 59 herein).
      1 GATGAATATA AATCCTTATT TTCTCTTCAT AGATGTGCCC ATACAGGCAG CAATTTCAAC
    61 AACATTCCCA TACACTGGTG TTCCCCCTTA TTCCCATGGA ACGGGAACAG GCTACACAAT
  121 AGACACAGTG ATCAGAACAC ATGAGTACTC AAACAAGGGG AAACAGTACA TTTCTGATGT
  181 TACAGGATGC ACAATGGTAG ATCCAACAAA TGGACCATTA CCCGAAGATA ATGAGCCGAG
  241 TGCCTATGCG CAATTAGATT GCGTTTTGGA GGCTTTGGAT AGAATGGATG AAGAACACCC
  301 AGGTCTGTTT CAAGCAGCCT CACAGAATGC TATGGAGGCC CTAATGGTCA CAACTGTAGA
  361 CAAATTAACC CAGGGGAGAC AGACTTTTGA TTGGACAGTA TGCAGAAACC AACCTGCTGC
  421 AACGGCACTG AACACAACAA TAACCTCTTT TAGGTTGAAT GATTTAAATG GAGCCGACAA
  481 AGGTGGATTA GTACCTTTTT GCCAGGATAT CATTGATTCA TTAGACAGAC CTGAAATGAC
  541 TTTCTTCTCA GTAAAGAATA TAAAGAAAAA ATTGCCTGCC AAAAACAGAA AGGGTTTCCT
  601 CATAAAGAGG ATACCAATGA AGGTAAAAGA CAAAATAACC AAAGTGGAAT ACATCAAAAG
  661 AGCATTATCA TTAAACACAA TGACAAAAGA CGCTGAAAGA GGCAAACTAA AAAGAAGAGC
  721 GATTGCCACC GCTGGAATAC AAATCAGAGG ATTTGTATTA GTAGTTGAAA ACTTGGCTAA
  781 AAATATATGT GAAAATCTAG AACAAAGTGG TTTACCAGTA GGTGGAAACG AGAAGAAAGC
  841 CAAACTGTCA AACGCAGTGG CCAAAATGCT CAGTAACTGC CCACCAGGAG GGATCAGCAT
  901 GACAGTAACA GGAGACAATA CTAAATGGAA TGAATGTTTA AACCCAAGAA TCTTTTTGGC
  961 TATGACTGAA AGAATAACCA GAGACAGCCC AATTTGGTTC AGGGATTTTT GTAGTATAGC
1021 ACCGGTCCTG TTCTCCAATA AGATAGCCAG ATTGGGGAAA GGGTTTATGA TAACAAGCAA
1081 AACAAAAAGA CTGAAGGCTC AAATACCTTG TCCTGATCTG TTTAGTATAC CATTAGAAAG
1141 ATATAATGAA GAAACAAGGG CAAAATTGAA AAAGCTAAAA CCATTCTTCA ATGAAGAAGG
1201 AACTGCATCT TTGTCGCCTG GGATGATGAT GGGAATGTTT AACATGCTAT CTACCGTGTT
1261 GGGAGTAGCC GCACTAGGTA TCAAGAACAT TGGAAACAAA GAATACTTAT GGGATGGACT
1321 GCAATCTTCT GATGATTTTG CTCTATTTGT TAATGCAAAG GATGAAGAAA CATGTATGGA
1381 AGGAATAAAC GACTTTTACC GAACATGTAA ATTATTGGGA ATAAACATGA GCAAAAAGAA
1441 AAGTTACTGT AATGAGACTG GAATGTTTGA ATTTACAAGC ATGTTCTACA GAGATGGATT
1501 TGTATCTAAT TTTGCAATGG AACTCCCTTC ATTTGGGGTT GCTGGAGTAA ATGAATCAGC
1561 AGATATGGCA ATAGGGATGA CAATAATAAA GAACAACATG ATCAACAATG GAATGGGTCC
1621 GGCAACAGCA CAAACAGCCA TACAGTTATT CATAGCTGAT TATAGATACA CCTACAAATG
1681 CCACAGGGGA GATTCCAAAG TAGAAGGAAA GAGAATGAAA ATCATAAAGG AGTTATGGGA
1741 AAACACTAAA GGAAGAGATG GTCTATTAGT AGCAGATGGT GGGCCCAACA TTTACAATTT
1801 GAGAAACTTG CATATTCCAG AAATAGTATT AAAGTATAAC CTAATGGACC CTGAATACAA
1861 AGGGCGATTA CTTCATCCTC AAAATCCCTT TGTGGGACAT TTGTCTATTG AGGGCATCAA
1921 AGAGGCAGAT ATAACCCCAG CACATGGTCC AGTAAAGAAA ATGGACTACG ATGCGGTGTC
1981 TGGAACTCAT AGTTGGAGAA CCAAAAGAAA CAGATCTATA CTAAACACTG ATCAGAGGAA
2041 CATGATTCTT GAGGAACAAT GCTACGCTAA GTGTTGCAAC CTATTTGAGG CCTGTTTTAA
2101 CAGTGCATCA TACAGGAAGC CAGTGGGTCA ACATAGCATG CTTGAGGCTA TGGCCCACAG
2161 ATTAAGAATG GATGCACGAT TAGATTATGA ATCAGGAAGA ATGTCAAAGG ATGATTTTGA
2221 GAAAGCAATG GCTCACCTTG GTGAGATTGG GTACATATAA GCTTCGAAGA TGTCTATGGG
2281 GTTATTGGTC ATCATTGAAT ACATGCGATA CACAAATGAT TAAAATGA
Segment 2: CY018708.1 from the NCBI website (provided as SEQ ID NO: 60 herein).
      1 GATGACATTG GCCAAAATTG AATTGTTAAA ACAACTGCTA AGGGACAATG AAGCCAAAAC
    61 AGTTTTGAAG CAAACAACGG TAGACCAATA TAACATAATA AGAAAATTCA ATACATCAAG
  121 GATTGAAAAG AATCCTTCAC TAAGGATGAA GTGGGCCATG TGTTCTAATT TTCCCTTGGC
  181 TCTAACCAAG GGCGATATGG CAAATAGAAT CCCCTTGGAA TACAAAGGAA TACAACTTAA
  241 AACAAATGCT GAAGACATAG GAACCAAAGG CCAAATGTGC TCAATAGCAG CAGTTACTTG
  301 GTGGAATACA TATGGACCAA TAGGAGATAC TGAAGGTTTC GAAAGGGTCT ACGAAAGCTT
  361 TTTTCTCAGA AAAATGAGAC TTGACAACGC CACTTGGGGC CGAATAACTT TTGGCCCAGT
  421 TGAAAGAGTG AGAAAAAGGG TACTGCTAAA CCCTCTCACC AAGGAAATGC CTCCAGATGA
  481 GGCGAGCAAT GTGATAATGG AAATATTGTT CCCTAAAGAA GCAGGAATAC CAAGAGAATC
  541 CACTTGGATA CATAGGGAAC TGATAAAAGA AAAAAGAGAA AAATTGAAAG GAACAATGAT
  601 AACTCCAATC GTACTGGCAT ACATGCTTGA AAGAGAACTG GTTGCTCGAA GAAGATTCTT
  661 GCCAGTGGCA GGAGCAACAT CAGCTGAGTT CATAGAAATG CTACACTGCT TACAAGGTGA
  721 AAATTGGAGA CAAATATATC ACCCAGGAGG GAATAAATTA ACTGAGTCTA GGTCTCAATC
  781 AATGATAGTA GCTTGTAGAA AAATAATCAG AAGATCAATA GTCGCTTCAA ACCCACTGGA
  841 GCTAGCTGTA GAAATTGCAA ACAAGACTGT GATAGATACT GAACCTTTAA AGTCATGTCT
  901 GGCAGCCATA GACGGAGGTG ATGTAGCTTG TGACATAATA AGAGCTGCAT TAGGACTAAA
  961 GATCAGACAA AGACAAAGAT TTGGACGGCT TGAGCTAAAA AGAATATCAG GAAGAGGATT
1021 CAAAAATGAT GAAGAAATAT TAATAGGGAA CGGAACAATA CAGAAGATTG GAATATGGGA
1081 CGGGGAAGAG GAGTTCCATG TAAGATGTGG TGAATGCAGG GGAATATTAA AAAAGAGTAA
1141 AATGAAACTG GAAAAACTAC TGATAAATTC AGCCAAAAAG GAGGATATGA GAGATTTAAT
1201 AATCTTATGC ATGGTATTTT CTCAAGACAC TAGGATGTTC CAAGGGGTGA GAGGAGAAAT
1261 AAATTTTCTT AATCGAGCAG GCCAACTTTT ATCTCCAATG TACCAACTCC AACGATATTT
1321 TTTGAATAGG AGCAACGACC TTTTTGATCA ATGGGGGTAT GAGGAATCAC CCAAAGCAAG
1381 TGAACTACAT GGGATAAATG AATCAATGAA TGCATCTGAC TATACATTGA AAGGGGTTGT
1441 AGTGACAAGA AATGTAATTG ACGACTTTAG CTCTACTGAA ACAGAAAAAG TATCCATAAC
1501 AAAAAATCTT AGTTTAATAA AAAGGACTGG GGAAGTCATA ATGGGAGCTA ATGACGTGAG
1561 TGAATTAGAA TCACAAGCAC AGCTGATGAT AACATATGAT ACACCTAAGA TGTGGGAAAT
1621 GGGAACAACC AAAGAACTGG TGCAAAACAC TTATCAATGG GTGCTAAAAA ACTTGGTAAC
1681 ACTGAAGGCT CAGTTTCTTC TAGGAAAAGA GGACATGTTC CAATGGGATG CATTTGAAGC
1741 ATTTGAGAGC ATAATTCCTC AGAAAATGGC TGGTCAGTAC AGTGGATTTG CAAGAGCAGT
1801 GCTCAAACAA ATGAGAGACC AGGAGGTTAT GAAAACTGAC CAGTTCATAA AGTTGTTGCC
1861 TTTTTGTTTC TCACCACCAA AATTAAGGAG CAATGGGGAG CCTTATCAAT TCTTAAAACT
1921 TGTATTGAAA GGAGGAGGGG AAAATTTCAT CGAAGTAAGG AAAGGGTCCC CTCTATTTTC
1981 CTATAATCCA CAAACAGAGG TCCTAACTAT ATGCGGCAGA ATGATGTCAT TAAAAGGGAA
2041 AATTGAAGAT GAAGAAAGGA ATAGATCAAT GGGGAATGCA GTATTAGCAG GCTTTCTCGT
2101 TAGTGGCAAG TATGACCCAG ATCTTGGAGA TTTCAAAACT ATTGAAGAAC TTGAAAAGCT
2161 GAAACCGGGG GAAAAGGCAA ACATCTTACT TTATCAAGGA AAGCCAGTTA AAGTAGTTAA
2221 AAGGAAAAGG TATAGTGCTT TGTCCAATGA CATTTCACAA GGAATTAAGA GACAAAGAAT
2281 GACAGTTGAG TCCATGGGGT GGGCCTTGAG CTAATATAAA TTTATCCATT AATTCAATGA
2341 ACGCAATTGA GT
Segment 3: CY018706.1 from the NCBI website (provided as SEQ ID NO: 61 herein).
      1 TTTGATTTGT CATAATGGAT ACTTTTATTA CAAGAAACTT CCAGACTACA ATAATACAAA
    61 AGGCCAAAAA CACAATGGCA GAATTTAGTG AAGATCCTGA ATTACAACCA GCAATGCTAT
  121 TCAATATCTG CGTCCATCTA GAGGTTTGCT ATGTAATAAG TGACATGAAT TTTCTTGACG
  181 AAGAAGGAAA AGCATATACA GCATTAGAAG GACAAGGGAA AGAACAAAAT TTGAGACCAC
  241 AATATGAAGT AATTGAGGGA ATGCCAAGAA CCATAGCATG GATGGTCCAA AGATCCTTAG
  301 CTCAAGAGCA TGGAATAGAG ACTCCCAAGT ATCTGGCTGA TTTGTTTGAT TATAAAACCA
  361 AGAGATTTAT AGAAGTTGGA ATAACAAAAG GATTGGCTGA TGATTACTTT TGGAAAAAGA
  421 AAGAAAAGTT GGGAAATAGC ATGGAACTGA TGATATTCAG CTACAATCAA GACTACTCGT
  481 TAAGTAATGA ATCCTCATTG GATGAGGAAG GGAAAGGGAG AGTGCTAAGC AGACTCACAG
  541 AACTTCAGGC TGAATTAAGT CTGAAAAACC TATGGCAAGT TCTCATAGGA GAAGAAGATG
  601 TTGAAAAGGG AATTGACTTT AAACTTGGAC AAACAATATC TAGACTAAGG GATATATCTG
  661 TTCCAGCTGG TTTCTCCAAT TTTGAAGGAA TGAGGAGCTA CATAGACAAT ATAGACCCAA
  721 AAGGAGCAAT AGAGAGAAAT CTAGCAAGGA TGTCTCCCTT AGTATCAGTC ACACCTAAAA
  781 AGTTAACATG GGAGGACCTA AGACCAATAG GGCCTCACAT TTACAACCAT GAGCTACCAG
  841 AAGTTCCATA TAATGCCTTT CTTCTAATGT CTGATGAACT GGGGCTGGCC AATATGACTG
  901 AGGGAAAGTC CAAAAAACCG AAGACATTAG CCAAAGAATG TCTAGAAAAG TACTCAACAC
  961 TACGGGATCA AACTGACCCA ATATTAATAA TGAAAAGCGA AAAAGCTAAC GAAAATTTCC
1021 TATGGAAGCT TTGGAGAGAC TGTGTAAATA CAATAAGTAA TGAGGAAATG AATAACGAGT
1081 TACAGAAAAC CAATTATGCC AAGTGGGCCA CAGGGGATGG ATTAACATAC CAGAAAATAA
1141 TGAAAGAAGT AGCAATAGAT GACGAAACAA TGTGCCAAGA AGAGCCTAAA ATCCCTAACA
1201 AATGTAGAGT GGCTGCTTGG GTTCAAACAG AGATGAATCT ATTGAGCACT CTGACAAGTA
1261 AAAGAGCTCT GGACCTACCA GAAATAGGGC CAGACGTAGC ACCCGTGGAG CATGTAGGGA
1321 GTGAAAGAAG GAAATACTTT GTTAATGAAA TCAACTACTG TAAGGCCTCT ACAGTTATGA
1381 TGAAGTATGT GCTTTTTCAC ACTTCATTGT TGAATGAAAG CAATGCCAGC ATGGGAAAAT
1441 ACAAAGTAAT ACCAATAACC AATAGAGTAG TAAATGAAAA AGGAGAAAGT TTCGACATGC
1501 TTTATGGTCT GGCGGTTAAA GGACAATCTC ATCTGAGGGG AGATACTGAT GTTGTAACAG
1561 TTGTAACTTT CGAATTTAGT AGTACAGACC CAAGAGTGGA CTCAGGAAAG TGGCCAAAAT
1621 ATACTGTGTT TAGGATTGGC TCCCTATTTG TGAGTGGGAG GGAAAAATCT GTGTACCTGT
1681 ATTGCCGAGT GAATGGCACA AATAAGATCC AAATGAAATG GGGAATGGAA GCTAGAAGAT
1741 GTCTGCTTCA ATCAATGCAA CAAATGGAAG CAATTGTTGA ACAGGAATCA TCGATACAAG
1801 GATATGACAT GACCAAAGCT TGTTTCAAGG GAGACAGAGT AAATAGCCCC AAAACTTTCA
1861 GTATTGGAAC TCAAGAAGGA AAACTAGTAA AAGGATCCTT TGGAAAAGCA CTAAGAGTAA
1921 TATTTACTAA ATGTTTGATG CACTATGTAT TTGGAAATGC CCAATTGGAG GGGTTTAGTG
1981 CCGAGTCTAG GAGACTTCTA CTGTTGATTC AAGCATTAAA GGACAGAAAG GGCCCTTGGG
2041 TGTTCGACTT AGAGGGAATG TATTCTGGAA TAGAAGAATG TATTAGTAAC AACCCTTGGG
2101 TAATACAGAG TGCATACTGG TTCAATGAAT GGTTGGGCTT TGAAAAGGAG GGGAGTAAAG
2161 TGTTAGAATC AGTGGATGAA ATAATGGATG AATAAAAGGA CATGGTACTC AAT
Segment 4: CY018701.1 from the NCBI website (provided as SEQ ID NO: 62 herein).
      1 ATATCCACAA AATGAAGGCA ATAATTGTAC TACTCATGGT AGTAACATCC AATGCAGATC
    61 GAATCTGCAC TGGGATAACA TCGTCAAACT CACCCCATGT GGTCAAAACT GCTACTCAAG
  121 GGGAGGTCAA TGTGACTGGT GTGATACCAC TGACAACAAC ACCCACCAAA TCTCATTTTG
  181 CAAATCTCAA AGGAACAAAA ACCAGAGGGA AACTATGCCC AAAATGCCTC AACTGCACAG
  241 ATCTGGACGT GGCCTTGGGC AGACCAAAAT GCACGGGGAA CATACCCTCG GCAAAAGTTT
  301 CAATACTCCA TGAAGTCAGA CCTGTTACAT CTGGGTGCTT TCCTATAATG CACGACAGAA
  361 CAAAAATTAG ACAGCTGCCC AATCTTCTCA GAGGATACGA ACATATCAGG TTATCAACTC
  421 ATAACGTTAT CAATGCAGAA AAGGCACCAG GAGGACCCTA CAAAATTGGA ACCTCAGGGT
  481 CTTGCCCTAA CGTTACCAAT GGAAACGGAT TTTTCGCAAC AATGGCTTGG GCCGTCCCAA
  541 AAAACGACAA CAACAAAACA GCAACAAATT CATTAACAAT AGAAGTACCA TACATTTGTA
  601 CAGAAGGAGA AGACCAAATT ACCGTTTGGG GGTTCCACTC TGATAACGAA GCCCAAATGG
  661 CAAAACTCTA TGGGGACTCA AAGCCCCAGA AGTTCACCTC ATCTGCCAAC GGAGTGACCA
  721 CACATTACGT TTCACAGATT GGTGGCTTCC CAAATCAAAC AGAAGACGGA GGACTACCAC
  781 AAAGTGGTAG AATTGTTGTT GATTACATGG TGCAAAAATC TGGGAAAACA GGAACAATTA
  841 CCTATCAAAG AGGTATTTTA TTGCCTCAAA AAGTGTGGTG CGCAAGTGGC AGGAGCAAGG
  901 TAATAAAAGG ATCCTTGCCT TTAATTGGAG AAGCAGATTG CCTCCACGAA AAATACGGTG
  961 GATTAAACAA AAGCAAGCCT TACTACACAG GGGAACATGC AAAGGCCATA GGAAATTGCC
1021 CAATATGGGT GAAAACACCC TTGAAGCTGG CCAATGGAAC CAAATATAGA CCTCCTGCAA
1081 AACTATTAAA GGAAAGAGGT TTCTTCGGAG CTATTGCTGG TTTCTTAGAA GGAGGATGGG
1141 AAGGAATGAT TGCAGGTTGG CACGGATACA CATCCCATGG GGCACATGGA GTAGCAGTGG
1201 CAGCAGACCT TAAGAGTACT CAAGAAGCCA TAAACAAGAT AACAAAAAAT CTCAACTCTT
1261 TGAGTGAGCT GGAAGTAAAG AATCTTCAAA GACTAAGCGG TGCCATGGAT GAACTCCACA
1321 ACGAAATACT AGAACTAGAC GAGAAAGTGG ATGATCTCAG AGCTGATACA ATAAGCTCAC
1381 AAATAGAACT CGCAGTCTTG CTTTCCAATG AAGGAATAAT AAACAGTGAA GATGAGCATC
1441 TCTTGGCGCT TGAAAGAAAG CTGAAGAAAA TGCTGGGCCC CTCTGCTGTA GAGATAGGGA
1501 ATGGATGCTT CGAAACCAAA CACAAGTGCA ACCAGACCTG TCTCGACAGA ATAGCTGCTG
1561 GTACCTTTGA TGCAGGAGAA TTTTCTCTCC CCACTTTTGA TTCACTGAAT ATTACTGCTG
1621 CATCTTTAAA TGACGATGGA TTGGATAATC ATACTATACT GCTTTACTAC TCAACTGCTG
1681 CCTCCAGTTT GGCTGTAACA TTGATGATAG CTATCTTTGT TGTTTATATG GTCTCCAGAG
1741 ACAATGTTTC TTGCTCCATC TGTCTATAAG GAAAGTTAAG CCCTGTATTT TCCTTTATTG
1801 TAGTGCTTGT TTGCTTGTTA CCATTACAAA AAAACGTTAT TGA
Segment 5: CY018704.1 from the NCBI website (provided as SEQ ID NO: 63 herein).
      1 TTTCTTGTGA ACTTCAAGTG CTAACAAAAG AACTGAAAAT CAAAATGTCC AACATGGATA
    61 TTGACGGTAT CAACACTGGG ACAATTGACA AAGCACCGGA AGAAATAACT TCTGGAACCA
  121 GTGGGACAAC CAGACCAATC ATCAGACCAG CAACCCTTGC CCCACCAAGC AACAAACGAA
  181 CCCGGAACCC ATCCCCGGAA AGAGCAACCA CAATCAGTGA AGCTGATGTC GGAAGGAAAA
  241 ACCAAAAGAA ACAGACCCCG ACAGAGATAA AGAAGAGCGT CTACAATATG GTAGTGAAAC
  301 TGGGTGAATT CTATAACCAG ATGATGGTCA AAGCTGGACT TAACGATGAC ATGGAGAGAA
  361 ACCTAATTCA AAATGCGCAT GCTGTGGAAA GAATTCTATT GGCTGCCACT GATGACAAGA
  421 AAACTGAATT CCAGAAGAAA AAGAATGCCA GAGATGTCAA AGAAGGGAAA GAAGAAATAG
  481 ATCACAACAA AACAGGGGGC ACCTTTTACA AGATGGTAAG AGATGATAAA ACCATCTACT
  541 TCAGCCCTAT AAGAGTCACC TTTTTAAAAG AAGAGGTAAA AACAATGTAC AAAACCACCA
  601 TGGGGAGTGA TGGCTTCAGC GGACTAAATC ACATAATGAT TGGGCATTCA CAGATGAATG
  661 ATGTCTGTTT CCAAAGATCA AAGGCACTAA AAAGAGTTGG ACTTGACCCT TCATTAATCA
  721 GTACCTTTGC AGGAAGCACA CTCCCCAGAA GATCAGGTGC AACTGGTGTT GCGATCAAAG
  781 GAGGTGGAAC TCTAGTGGCT GAAGCCATTC GATTTATAGG AAGAGCAATG GCAGACAGAG
  841 GGCTATTGAG AGACATCAAA GCTAAGACTG CTTATGAAAA GATTCTTCTG AATCTAAAAA
  901 ACAAATGCTC TGCGCCCCAA CAAAAGGCTC TAGTTGATCA AGTGATCGGA AGTAGAAATC
  961 CAGGGATCGC AGACATTGAA GACCTAACCC TGCTTGCTCG TAGTATGGTC GTTGTTAGGC
1021 CCTCTGTGGC GAGCAAAGTA GTGCTTCCCA TAAGCATTTA CGCCAAAATA CCTCAACTAG
1081 GGTTCAACGT TGAAGAGTAC TCTATGGTTG GGTATGAAGC CATGGCTCTT TACAATATGG
1141 CAACACCTGT TTCCATATTA AGAGTGGGAG ATGATGCAAA AGACAAATCA CAATTATTCT
1201 TCATGTCTTG CTTCGGAGCT GCCTATGAAG ACCTGAGAGT TTTGTCTGCA TTAACAGGCA
1261 CAGAGTTCAA GCCTAGATCA GCATTAAAAT GCAAGGGTTT CCATGTTCCA GCAAAGGAAC
1321 AGGTGGAAGG AATGGGGGCA GCTCTGATGT CCATCAAGCT CCAGTTTTGG GCTCCAATGA
1381 CCAGATCTGG GGGGAACGAA GTAGGTGGAG ACGGGGGGTC TGGCCAAATA AGTTGCAGCC
1441 CAGTGTTTGC AGTAGAAAGA CCTATTGCTC TAAGCAAGCA AGCTGTAAGA AGAATGCTGT
1501 CAATGAATAT TGAGGGACGT GATGCAGATG TCAAAGGAAA TCTACTCAAG ATGATGAATG
1561 ACTCAATGGC TAAGAAAGCC AATGGAAATG CTTTCATTGG GAAGAAAATG TTTCAAATAT
1621 CAGACAAAAA CAAAACCAAT CCCGTTGAAA TTCCAATTAA GCAAACCATC CCCAATTTCT
1681 TCTTTGGGAG GGACACAGCA GAGGATTATG ATGACCTCGA TTATTAAAGC AACAAAATAG
1741 ACACTATGAC TGTGATTGTT TCAATACGTT TGGAATGTGG GTGTTTACTC TTATTAAAAT
1801 AAATATAAA
Segment 6: CY018703.1 from the NCBI website (provided as SEQ ID NO: 64 herein).
      1 AAACTGAGGC AAATAGGCCA AAAATGAACA ATGCTACCTT CAACTATACA AACGTTAACC
    61 CTATTTCTCA CATCAGGGGG AGTATTATTA TCACTATATG TGTCAGCTTC ATTGTCATAC
  121 TTACTATATT CGGATATATT GCTAAAATTC TCACCAACAG AAATAACTGC ACCAACAATG
  181 CCATTGGATT GTGCAAACGC ATCAAATGTT CAGGCTGTGA ACCGTTCTGC AACAAAAGGG
  241 GTGACACTTC TTCTCCCAGA ACCAGAGTGG ACATACCCGC GTTTATCTTG CCCGGGCTCA
  301 ACCTTTCAGA AAGCACTCCT AATTAGCCCT CATAGATTCG GAGAAACCAA AGGAAACTCA
  361 GCTCCCTTGA TAATAAGGGA ACCTTTTATT GCTTGTGGAC CAAAGGAATG CAAACACTTT
  421 GCTCTAACCC ATTATGCAGC CCAACCAGGG GGATACTACA ATGGAACAAG AGGAGACAGA
  481 AACAAGCTGA GGCATCTAAT TTCAGTCAAA TTGGGCAAAA TCCCAACAGT AGAAAACTCC
  541 ATTTTCCACA TGGCAGCATG GAGCGGGTCC GCATGCCATG ATGGTAAAGA ATGGACATAT
  601 ATCGGAGTTG ATGGCCCTGA CAATAATGCA TTGCTCAAAA TAAAATATGG AGAAGCATAT
  661 ACTGACACAT ACCATTCCTA TGCAAACAAC ATCCTAAGAA CACAAGAAAG TGCCTGCAAT
  721 TGCATCGGGG GAAATTGTTA TCTTATGATA ACTGATGGCT CAGCTTCAGG TATTAGTGAA
  781 TGCAGATTTC TTAAAATTCG AGAGGGCCGA ATAATAAAAG AAATATTTCC AACAGGAAGA
  841 GTAAAACATA CTGAAGAATG CACATGCGGA TTTGCCAGCA ATAAGACCAT AGAATGTGCC
  901 TGTAGAGATA ACAGTTACAC AGCAAAAAGA CCCTTTGTCA AATTAAACGT GGAGACTGAT
  961 ACAGCAGAAA TAAGATTGAT GTGCACAGAG ACTTATTTGG ACACCCCCAG ACCAGATGAT
1021 GGAAGCATAA CAGGGCCTTG TGAATCTAAT GGGGACAAAG GGAGTGGAGG CATCAAGGGA
1081 GGATTTGTTC ATCAAAGAAT GGCATCCAAG ATTGGAAGGT GGTACTCTCG AACGATGTCT
1141 AAAACTAAAA GGATGGGGAT GGGACTGTAT GTCAAGTATG ATGGAGACCC ATGGGCTGAC
1201 AGTGATGCCC TTGCTCTTAG TGGAGTAATG GTTTCAATGG AAGAACCTGG TTGGTACTCC
1261 TTTGGCTTCG AAATAAAAGA TAAGAAATGT GATGTCCCCT GTATTGGAAT AGAGATGGTA
1321 CATGATGGTG GAAAAGAGAC TTGGCACTCA GCAGCAACAG CCATTTACTG TTTAATGGGC
1381 TCAGGACAGC TGCTGTGGGA CACTGTCACA GGTGTTGATA TGGCTCTGTA ATGGAGGAAT
1441 GGTTGAGTCT GTTCTAAACC CTTTGTTCCT ATTTTGTTTG AACAATTGTC CTTACTGAAC
1501 TTAATTGTTT CTGAAA
Segment 7: CY018702.1 from the NCBI website (provided as SEQ ID NO: 65 herein).
      1 AAAATGTCGC TGTTTGGAGA CACAATTGCC TACCTGCTTT CATTGACAGA AGATGGAGAA
    61 GGCAAAGCAG AACTAGCAGA AAAATTACAC TGTTGGTTTG GTGGGAAAGA ATTTGACCTA
  121 GACTCTGCCT TGGAATGGAT AAAAAACAAA AGATGCTTAA CTGATATACA AAAAGCACTA
  181 ATTGGTGCCT CTATCTGCTT TTTAAAACCC AAAGACCAGG AAAGAAAAAG AAGATTCATC
  241 ACAGAGCCCT TATCAGGAAT GGGAACAACA GCAACAAAAA AGAAAGGCCT GATTCTGGCT
  301 GAGAGAAAAA TGAGAAGATG TGTGAGCTTT CATGAAGCAT TTGAAATAGC AGAAGGCCAT
  361 GAAAGCTCAG CGCTACTATA CTGTCTCATG GTCATGTACC TGAATCCTGG AAATTATTCA
  421 ATGCAAGTAA AACTAGGAAC GCTCTGTGCT TTGTGCGAGA AACAAGCATC ACATTCACAC
  481 AGGGCTCATA GCAGAGCAGC GAGATCTTCA GTGCCTGGAG TGAGACGAGA AATGCAGATG
  541 GTCTCAGCTA TGAACACAGC AAAAACAATG AATGGAATGG GAAAAGGAGA AGACGTCCAA
  601 AAGCTGGCAG AAGAGCTGCA AAGCAACATT GGAGTGCTGA GATCTCTTGG GGCAAGTCAA
  661 AAGAATGGGG AAGGAATTGC AAAGGATGTA ATGGAGGTGC TAAAGCAGAG CTCTATGGGA
  721 AATTCAGCTC TTGTGAAGAA ATATCTATAA TGCTCGAACC ATTTCAGATT CTTTCAATTT
  781 GTTCTTTTAT CTTATCAGCT CTCCATTTCA TGGCTTGGAC AATAGGGCAT TTGAATCAAA
  841 TAAAAAGAGG AGTAAACATG AAGATACGAA TAAAAAGTCC AAACAAAGAG ACAATAAACA
  901 GAGAGGTATC AATTTTGAGA CACAGTTACC AAAAAGAAAT CCAGGCCAAA GAAACAATGA
  961 AGGAAGTACT CTCTGACAAC ATGGAGGTAT TGAGTGACCA CATGGTGATT GAGGGGCTTT
1021 CTGCCGAAGA GATAATAAAA ATGGGTGAAA CAGTTTTGGA GATAGAAGAA TTGCATTAAA
1081 TTCAATTTTT ACTGTATTTC TTACCATGCA TTTAAGCAAA TTGTAATCAA TGTCAGCAAA
1141 TAAACT
Segment 8: CY018705.1 from the NCBI website (provided as SEQ ID NO: 66 herein).
      1 TCACTGGCAA ACAGGAAAAA TGGCGAACAA CATGACCACA ACACAAATTG AGGTGGGTCC
    61 GGGAGCAACC AATGCCACCA TAAACTTTGA AGCAGGAATT CTGGAGTGCT ATGAAAGGCT
  121 TTCATGGCAA AGAGCCCTTG ACTACCCTGG ACAAGACCGC CTAAACAGAC TAAAGAGAAA
  181 ATTAGAGTCA AGAATAAAGA CTCACAACAA AAGTGAGCCT GAAAGTAAAA GGATGTCCCT
  241 TGAAGAGAGA AAAGCAATTG GAGTAAAAAT GATGAAAGTA CTCCTATTTA TGAATCCGTC
  301 TGCTGGAATT GAAGGGTTTG AGCCATACTG TATGAAAAGT TCCTCAAATA GCAACTGTAC
  361 GAAATACAAT TGGACCGATT ACCCTTCAAC ACCAGGGAGG TGCCTTGATG ACATAGAAGA
  421 AGAACCAGAG GATGTTGATG GCCCAACTGA AATAGTATTA AGGGACATGA ACAACAAAGA
  481 TGCAAGGCAA AAGATAAAGG AGGAAGTAAA CACTCAGAAA GAAGGGAAGT TCCGTTTGAC
  541 AATAAAAAGG GATATGCGTA ATGTATTGTC CTTGAGAGTG TTGGTAAACG GAACATTCCT
  601 CAAACACCCC AATGGATACA AGTCCTTATC AACTCTGCAT AGATTGAATG CATATGACCA
  661 GAGTGGAAGG CTTGTTGCTA AACTTGTTGC TACTGATGAT CTTACAGTGG AGGATGAAGA
  721 AGATGGCCAT CGGATCCTCA ACTCACTCTT CGAGCGTCTT AATGAAGGAC ATTCAAAGCC
  781 AATTCGAGCA GCTGAAACTG CGGTGGGAGT CTTATCCCAA TTTGGTCAAG AGCACCGATT
  841 ATCACCAGAA GAGGGAGACA ATTAGACTGG TCACGGAAGA ACTTTATCTT TTAAGTAAAA
  901 GAATTGATGA TAACATATTG TTCCACAAAA CAGTAATAGC TAACAGCTCC ATAATAGCTG
  961 ACATGGTTGT ATCATTATCA TTATTAGAAA CATTGTATGA AATGAAGGAT GTGGTTGAAG
1021 TGTACAGCAG GCAGTGCTTG TGAATTTAAA ATAAA
```

The Influenza viral genome is RNA. Hence, in some cases the Influenza viral genome can be a copy of the foregoing DNA sequence, where the thymine (T) residues are uracil (U) residues. In some cases, the Influenza viral genome can be a complement of the foregoing DNA sequence.

However, the Influenza viral genome can also have sequence variation. For example, the Influenza viral genome can be for various Influenza strains including the foregoing sequence for strain H1N1, or other strains such as H3N2, or any of the Influenza A 18 distinct subtypes of hemagglutinin (HA) and 11 distinct subtypes of neuraminidase (NA). Variations in the Influenza B virus can be any of strains B/Lee/1940, B/Brisbane/60/2008, B/Victoria/504/2000, or other strains.

Sequencing has confirmed that Influenza viruses share a common genetic ancestry; however, they have genetically diverged, such that reassortment—the exchange of viral RNA segments between viruses—has been reported to occur within each genus, or type, but not across types. This genetic reassortment has led to a standard naming convention for Influenza viruses that includes virus type; species from which it was isolated (if non-human); location at which it was isolated; isolate number; isolate year; and, for influenza A viruses only, HA and NA subtype. In Influenza A and B viruses, genome segments 1, 3, 4, and 5 encode just one protein per segment: the PB2, PA, HA and NP proteins. All Influenza viruses encode the polymerase subunit PB1 on segment 2; in some strains of Influenza A virus, this segment also codes for the accessory protein PB1-F2, a small, 87-amino acid protein with pro-apoptotic activity, in a +1 alternate reading frame. No analogue to PB1-F2 has been identified in influenza B or C viruses. Conversely, segment 6 of the Influenza A virus encodes only the NA protein, while that of Influenza B virus encodes both the NA protein and, in a −1 alternate reading frame, the NB matrix protein, which is an integral membrane protein corresponding to the influenza A virus M2 protein. Segment 7 of both influenza A and B viruses code for the M1 matrix protein. In the influenza A genome, the M2 ion channel is also expressed from segment 7 by RNA splicing, while influenza B virus encodes its BM2 membrane protein in a +2 alternate reading frame. Finally, both influenza A and B viruses possess a single RNA segment, segment 8, from which they express the interferon-antagonist NS1 protein and, by mRNA splicing, the NEP/NS2, which is involved in viral RNP export from the host cell nucleus. The genomic organization of influenza C viruses is generally similar to that of influenza A and B viruses; however, the HEF protein of influenza C replaces the HA and NA proteins, and thus the influenza C virus genome has one fewer segment than that of influenza A or B viruses.

### Cas13 Protein:

Any suitable CRISPR-associated RNA-targeting endonuclease, such as a Cas13 protein variant, can be used in the methods and compositions described herein. The Cas13 protein can complex with at least one CRISPR guide RNA (crRNA) to at least one reporter RNA for a period of time sufficient to form at least one RNA cleavage product.

The Cas13 protein can, for example, be a Cas13a protein, Cas13b protein, or a combination thereof. Cas13 contains two Higher Eukaryotes and Prokaryotes Nucleotide-binding (HEPN) domains for RNA cleavage, consistent with known roles for HEPN domains in other proteins. In some embodiments, the Cas13 proteins can have sequence variation and/or be from other organisms. For example, the Cas13 proteins can have at least about 75%, at least about 80%, at least about 85%, at least about 90%, at least about 95%, at least about 96%, at least about 97%, at least about 98%, at least about 99%, or about 100% sequence identity to any of the foregoing Cas 13 sequences or to a Cas13 in the following bacteria: Leptotrichia wadei, Leptotrichia buccalis, Rhodobacter capsulatus, Herbinix hemicellulosilytica, Leptotrichia buccalis (Lbu), Listeria seeligeri, Paludibacter propionicigenes, Lachnospiraceae bacterium, [Eubacterium] rectale, Listeria newyorkensis, Clostridium aminophilum, and/or Leptotrichia shahii.

For example, a Leptotrichia wadei Cas13a endonuclease can be used that has the following sequence (SEQ ID NO: 38; NCBI accession no. WP_036059678.1).

```
      1 MKITKIDGVS HYKKQDKGIL KKKWKDLDER KQREKIEARY
    41 NKQIESKIYK EFFRLKNKKR IEKEEDQNIK SLYFFIKELY
    81 LNEKNEEWEL KNINLEILDD KERVIKGYKF KEDVYFFKEG
  121 YKEYYLRILF NNLIEKVQNE NREKVRKNKE FLDLKEIFKK
  161 YKNRKIDLLL KSINNNKINL EYKKENVNEE IYGINPTNDR
  201 EMTFYELLKE IIEKKDEQKS ILEEKLDNED ITNFLENIEK
  241 IFNEETEINI IKGKVLNELR EYIKEKEENN SDNKLKQIYN
  281 LELKKYIENN FSYKKQKSKS KNGKNDYLYL NFLKKIMFIE
  321 EVDEKKEINK EKFKNKINSN FKNLFVQHIL DYGKLLYYKE
  361 NDEYIKNTGQ LETKDLEYIK TKETLIRKMA VLVSFAANSY
  401 YNLFGRVSGD ILGTEVVKSS KTNVIKVGSH IFKEKMLNYE
  441 FDFEIFDANK IVEILESISY SIYNVRNGVG HENKLILGKY
  481 KKKDINTNKR IEEDLNNNEE IKGYFIKKRG EIERKVKEKF
  521 LSNNLQYYYS KEKIENYFEV YEFEILKRKI PFAPNFKRII
  561 KKGEDLENNK NNKKYEYFKN FDKNSAEEKK EFLKTRNELL
  601 KELYYNNFYK EFLSKKEEFE KIVLEVKEEK KSRGNINNKK
  641 SGVSFQSIDD YDTKINISDY IASIHKKEME RVEKYNEEKQ
  681 KDTAKYIRDF VEEIFLTGFI NYLEKDKRLH FLKEEFSILC
  721 NNNNNVVDEN ININEEKIKE FLKENDSKTL NLYLFENMID
  761 SKRISEFRNE LVKYKQFTKK RLDEEKEFLG IKIELYETLI
  801 EFVILTREKL DTKKSEEIDA WLVDKLYVKD SNEYKEYEEI
  841 LKLFVDEKIL SSKEAPYYAT DNKTPILLSN FEKTRKYGTQ
  881 SFLSEIQSNY KYSKVEKENI EDYNKKEEIE QKKKSNIEKL
  921 QDLKVELHKK WEQNKITEKE IEKYNNTTRK INEYNYLKNK
  961 EELQNVYLLH EMLSDLLARN VAFFNKWERD FKFIVIAIKQ
1001 FLRENDKEKV NEFLNPPDNS KGKKVYFSVS KYKNTVENID
1041 GIHKNEMNLI FLNNKFMNRK IDKMNCAIWV YERNYIAHFL
1081 HLHTKNEKIS LISQMNLLIK LFSYDKKVQN HILKSTKTLL
1121 EKYNIQINFE ISNDKNEVEK YKIKNRLYSK KGKMLGKNNK
1161 LENEFLE NVKAMLEYSE
```

Other sequences for Leptotrichia wadei Cas13a endonucleases are also available, such as those NCBI accession nos. BBM46759.1, BBM48616.1, BBM48974.1, BBM48975.1, and WP_021746003.1.

In another example, a Herbinix hemicellulosilytica Cas13a endonuclease can be used that has the following sequence (SEQ ID NO. 39; NCBI accession no. WP_103203632.1).

```
      1 MKLTRRRISG NSVDQKITAA FYRDMSQGLL YYDSEDNDCT
    41 DKVIESMDFE RSWRGRILKN GEDDKNPFYM FVKGLVGSND
    81 KIVCEPIDVD SDPDNLDILI NKNLTGFGRN LKAPDSNDTL
  121 ENLIRKIQAG IPEEEVLPEL KKIKEMIQKD IVNRKEQLLK
  161 SIKNNRIPFS LEGSKLVPST KKMKWLFKLI DVPNKTENEK
  201 MLEKYWEIYD YDKLKANITN RLDKTDKKAR SISRAVSEEL
  241 REYHKNLRTN YNRFVSGDRP AAGLDNGGSA KYNPDKEEFL
  281 LFLKEVEQYF KKYFPVKSKH SNKSKDKSLV DKYKNYCSYK
  321 VVKKEVNRSI INQLVAGLIQ QGKLLYYFYY NDTWQEDELN
  361 SYGLSYIQVE EAFKKSVMTS LSWGINRLTS FFIDDSNTVK
  401 FDDITTKKAK EAIESNYENK LRTCSRMQDH FKEKLAFFYP
  441 VYVKDKKDRP DDDIENLIVL VKNAIESVSY LRNRTFHFKE
  481 SSLLELLKEL DDKNSGQNKI DYSVAAEFIK RDIENLYDVE
  521 REQIRSLGIA EYYKADMISD CFKTCGLEFA LYSPKNSLMP
  561 AFKNVYKRGA NLNKAYIRDK GPKETGDQGQ NSYKALEEYR
  601 ELTWYIEVKN NDQSYNAYKN LLQLIYYHAF LPEVRENEAL
  641 ITDFINRTKE WNRKETEERL NTKNNKKHKN FDENDDITVN
  681 TYRYESIPDY QGESLDDYLK VLQRKQMARA KEVNEKEEGN
  721 NNYIQFIRDV VVWAFGAYLE NKLKNYKNEL QPPLSKENIG
  761 LNDTLKELFP EEKVKSPENI KCRFSISTFI DNKGKSTDNT
  801 SAEAVKTDGK EDEKDKKNIK RKDLLCFYLF LRLLDENEIC
  841 KLQHQFIKYR CSLKERRFPG NRTKLEKETE LLAELEELME
  881 LVRFTMPSIP EISAKAESGY DTMIKKYFKD FIEKKVEKNP
  921 KTSNLYYHSD SKTPVTRKYM ALLMRSAPLH LYKDIFKGYY
  961 LITKKECLEY IKLSNIIKDY QNSLNELHEQ LERIKLKSEK
1001 QNGKDSLYLD KKDFYKVKEY VENLEQVARY KHLQHKINFE
1041 SLYRIFRIHV DIAARMVGYT QDWERDMHEL FKALVYNGVL
1081 EERRFEAIFN NNDDNNDGRI VKKIQNNLNN KNRELVSMLC
1121 WNKKLNKNEF GAIIWKRNPI AHLNHFTQTE QNSKSSLESL
1161 INSLRILLAY DRKRQNAVTK TINDLLLNDY HIRIKWEGRV
1201 DEGQIYFNIK EKEDIENEPI IHLKHLHKKD CYIYKNSYMF
1241 DKQKEWICNG IKEEVYDKSI LKCIGNLFKF DYEDKNKSSA
1281 NPKHT
```

However, in some cases the Cas13 proteins with the SEQ ID NO: 39 sequence are not used.

In another example, a Leptotrichia buccalis Cas13a endonuclease can be used that has the following sequence (SEQ ID NO: 40; NCBI accession no. WP_015770004.1).

```
      1 MKVTKVGGIS HKKYTSEGRL VKSESEENRT DERLSALLNM
    41 RLDMYIKNPS STETKENQKR IGKLKKFFSN KMVYLKDNTL
    81 SLKNGKKENI DREYSETDIL ESDVRDKKNF AVLKKIYLNE
  121 NVNSEELEVF RNDIKKKLNK INSLKYSFEK NKANYQKINE
  161 NNIEKVEGKS KRNIIYDYYR ESAKRDAYVS NVKEAFDKLY
  201 KEEDIAKLVL EIENLTKLEK YKIREFYHEI IGRKNDKENF
  241 AKIIYEEIQN VNNMKELIEK VPDMSELKKS QVFYKYYLDK
  281 EELNDKNIKY AFCHFVEIEM SQLLKNYVYK RLSNISNDKI
  321 KRIFEYQNLK KLIENKLINK LDTYVRNCGK YNYYLQDGEI
  361 ATSDFIARNR QNEAFLRNII GVSSVAYFSL RNILETENEN
  401 DITGRMRGKT VKNNKGEEKY VSGEVDKIYN ENKKNEVKEN
  441 LKMFYSYDEN MDNKNEIEDF FANIDEAISS IRHGIVHENL
  481 ELEGKDIFAF KNIAPSEISK KMFQNEINEK KLKLKIFRQL
  521 NSANVFRYLE KYKILNYLKR TRFEFVNKNI PFVPSFTKLY
  561 SRIDDLKNSL GIYWKTPKTN DDNKTKEIID AQIYLLKNIY
  601 YGEFLNYFMS NNGNFFEISK EIIELNKNDK RNLKTGFYKL
  641 QKFEDIQEKI PKEYLANIQS LYMINAGNQD EEEKDTYIDF
  681 IQKIFLKGFM TYLANNGRLS LIYIGSDEET NTSLAEKKQE
  721 FDKELKKYEQ NNNIKIPYEI NEFLREIKLG NILKYTERLN
  761 MFYLILKLLN HKELTNLKGS LEKYQSANKE EAFSDQLELI
  801 NLLNLDNNRV TEDFELEADE IGKFLDENGN KVKDNKELKK
  841 FDTNKIYFDG ENIIKHRAFY NIKKYGMLNL LEKIADKAGY
  881 KISIEELKKY SNKKNEIEKN HKMQENLHRK YARPRKDEKF
  921 TDEDYESYKQ AIENIEEYTH LKNKVEFNEL NLLQGLLLRI
  961 LHRLVGYTSI WERDLRERLK GEFPENQYIE EIFNFENKKN
1001 VKYKGGQIVE KYIKFYKELH QNDEVKINKY SSANIKVLKQ
1041 EKKDLYIRNY IAHFNYIPHA EISLLEVLEN LRKLLSYDRK
1081 LKNAVMKSVV DILKEYGFVA TFKIGADKKI GIQTLESEKI
1121 VHLKNLKKKK LMTDRNSEEL CKLVKIMFEY KMEEKKSEN
```

However, in some cases the Cas13 proteins with the SEQ ID NO: 40 sequence are not used.

In another example, a Leptotrichia seeligeri Cas13a endonuclease can be used that has the following sequence (SEQ ID NO: 41; NCBI accession no. WP_012985477.1).

```
      1 MWISIKTLIH HLGVLFFCDY MYNRREKKII EVKTMRITKV
    41 EVDRKKVLIS RDKNGGKLVY ENEMQDNTEQ IMHHKKSSFY
    81 KSVVNKTICR PEQKQMKKLV HGLLQENSQE KIKVSDVTKL
  121 NISNFLNHRF KKSLYYFPEN SPDKSEEYRI EINLSQLLED
  161 SLKKQQGTFI CWESFSKDME LYINWAENYI SSKTKLIKKS
  201 IRNNRIQSTE SRSGQLMDRY MKDILNKNKP FDIQSVSEKY
  241 QLEKLTSALK ATFKEAKKND KEINYKLKST LQNHERQIIE
  281 ELKENSELNQ FNIEIRKHLE TYFPIKKTNR KVGDIRNLEI
  321 GEIQKIVNHR LKNKIVQRIL QEGKLASYEI ESTVNSNSLQ
  361 KIKIEEAFAL KFINACLFAS NNLRNMVYPV CKKDILMIGE
  401 FKNSFKEIKH KKFIRQWSQF FSQEITVDDI ELASWGLRGA
  441 IAPIRNEIIH LKKHSWKKFF NNPTFKVKKS KIINGKTKDV
  481 TSEFLYKETL FKDYFYSELD SVPELIINKM ESSKILDYYS
  521 SDQLNQVFTI PNFELSLLTS AVPFAPSFKR VYLKGEDYQN
  561 QDEAQPDYNL KLNIYNEKAF NSEAFQAQYS LFKMVYYQVF
  601 LPQFTINNDL FKSSVDFILT LNKERKGYAK AFQDIRKMNK
  641 DEKPSEYMSY IQSQLMLYQK KQEEKEKINH FEKFINQVFI
  681 KGFNSFIEKN RLTYICHPTK NTVPENDNIE IPFHTDMDDS
  721 NIAFWLMCKL LDAKQLSELR NEMIKFSCSL QSTEEISTFT
  761 KAREVIGLAL LNGEKGCNDW KELFDDKEAW KKNMSLYVSE
  801 ELLQSLPYTQ EDGQTPVINR SIDLVKKYGT ETILEKLFSS
  841 SDDYKVSAKD IAKLHEYDVT EKIAQQESLH KQWIEKPGLA
  881 RDSAWTKKYQ NVINDISNYQ WAKTKVELTQ VRHLHQLTID
  921 LLSRLAGYMS IADRDFQFSS NYILERENSE YRVTSWILLS
  961 ENKNKNKYND YELYNLKNAS IKVSSKNDPQ LKVDLKQLRL
1001 TLEYLELFDN RLKEKRNNIS HFNYLNGQLG NSILELEDDA
1041 RDVLSYDRKL KNAVSKSLKE ILSSHGMEVT FKPLYQTNHH
1081 LKIDKLQPKK IHHLGEKSTV SSNQVSNEYC QLVRTLLTMK
```

For example, a Paludibacter propionicigenes Cas13a endonuclease can be used that has the following sequence (SEQ ID NO: 42; NCBI accession no. WP_013443710.1).

```
      1 MRVSKVKVKD GGKDKMVLVH RKTTGAQLVY SGQPVSNETS
    41 NILPEKKRQS FDLSTINKTI IKFDTAKKQK LNVDQYKIVE
    81 KIFKYPKQEL PKQIKAEEIL PFLNHKFQEP VKYWKNGKEE
  121 SFNLTLLIVE AVQAQDKRKL QPYYDWKTWY IQTKSDLLKK
  161 SIENNRIDLT ENLSKRKKAL LAWETEFTAS GSIDLTHYHK
  201 VYMTDVLCKM LQDVKPLTDD KGKINTNAYH RGLKKALQNH
  241 QPAIFGTREV PNEANRADNQ LSIYHLEVVK YLEHYFPIKT
  281 SKRRNTADDI AHYLKAQTLK TTIEKQLVNA IRANIIQQGK
  321 TNHHELKADT TSNDLIRIKT NEAFVLNLTG TCAFAANNIR
  361 NMVDNEQTND ILGKGDFIKS LLKDNTNSQL YSFFFGEGLS
  401 TNKAEKETQL WGIRGAVQQI RNNVNHYKKD ALKTVENISN
  441 FENPTITDPK QQTNYADTIY KARFINELEK IPEAFAQQLK
  481 TGGAVSYYTI ENLKSLLTTF QFSLCRSTIP FAPGFKKVEN
  521 GGINYQNAKQ DESFYELMLE QYLRKENFAE ESYNARYFML
  561 KLIYNNLFLP GFTTDRKAFA DSVGFVQMQN KKQAEKVNPR
  601 KKEAYAFEAV RPMTAADSIA DYMAYVQSEL MQEQNKKEEK
  641 VAEETRINFE KFVLQVFIKG FDSFLRAKEF DFVQMPQPQL
  681 TATASNQQKA DKLNQLEASI TADCKLTPQY AKADDATHIA
  721 FYVFCKLLDA AHLSNLRNEL IKFRESVNEF KFHHLLEIIE
  761 ICLLSADVVP TDYRDLYSSE ADCLARLRPF IEQGADITNW
  801 SDLFVQSDKH SPVIHANIEL SVKYGTTKLL EQIINKDTQF
  841 KTTEANFTAW NTAQKSIEQL IKQREDHHEQ WVKAKNADDK
  881 EKQERKREKS NFAQKFIEKH GDDYLDICDY INTYNWLDNK
  921 MHFVHLNRLH GLTIELLGRM AGFVALEDRD FQFFDEQQIA
  961 DEFKLHGFVN LHSIDKKLNE VPTKKIKEIY DIRNKIIQIN
1001 GNKINESVRA NLIQFISSKR NYYNNAFLHV SNDEIKEKQM
1041 YDIRNHIAHF NYLTKDAADF SLIDLINELR ELLHYDRKLK
1081 NAVSKAFIDL FDKHGMILKL KLNADHKLKV ESLEPKKIYH
1121 LGSSAKDKPE YQYCTNQVMM AYCNMCRSLL EMKK
```

For example, a Lachnospiraceae bacterium Cas13a endonuclease can be used that has the following sequence (SEQ ID NO: 43; NCBI accession no. WP_022785443.1).

```
      1 MKISKVREEN RGAKLTVNAK TAVVSENRSQ EGILYNDPSR
    41 YGKSRKNDED RDRYIESRLK SSGKLYRIEN EDKNKRETDE
    81 LQWFLSEIVK KINRRNGLVL SDMLSVDDRA FEKAFEKYAE
  121 LSYTNRRNKV SGSPAFETCG VDAATAERLK GIISETNFIN
  161 RIKNNIDNKV SEDIIDRIIA KYLKKSLCRE RVKRGLKKLL
  201 MNAFDLPYSD PDIDVQRDFI DYVLEDFYHV RAKSQVSRSI
  241 KNMNMPVQPE GDGKFAITVS KGGTESGNKR SAEKEAFKKE
  281 LSDYASLDER VRDDMLRRMR RLVVLYFYGS DDSKLSDVNE
  321 KFDVWEDHAA RRVDNREFIK LPLENKLANG KTDKDAERIR
  361 KNTVKELYRN QNIGCYRQAV KAVEEDNNGR YFDDKMLNME
  401 FIHRIEYGVE KIYANLKQVT EKIWKDLINY EKIWKDLINY
  441 ISIKYIAMGK AVYNYAMDEL NASDKKEIEL GKISEEYLSG
  481 ISSFDYELIK AEEMLQRETA VYVAFAARHL SSQTVELDSE
  521 NSDFLLLKPK GTMDKNDKNK LASNNILNFL KDKETLRDTI
  561 LQYFGGHSLW TDFPFDKYLA GGKDDVDELT DLKDVIYSMR
  601 NDSFHYATEN HNNGKWNKEL ISAMFEHETE RMTVVMKDKF
  641 YSNNLPMFYK NDDLKKLLID LYKDNVERAS QVPSFNKVFV
  681 RKNFPALVRD KDNLGIELDL KADADKGENE LKFYNALYYM
  721 FKEIYYNAFL NDKNVRERFI TKATKVADNY DRNKERNLKD
  761 RIKSAGSDEK KKLREQLQNY IAENDEGQRI KNIVQVNPDY
  801 TLAQICQLIM TEYNQQNNGC MQKKSAARKD INKDSYQHYK
  841 MLLLVNLRKA FLEFIKENYA FVLKPYKHDL CDKADFVPDF
  881 AKYVKPYAGL ISRVAGSSEL QKWYIVSREL SPAQANHMLG
  921 FLHSYKQYVW DIYRRASETG TEINHSIAED KIAGVDITDV
  961 DAVIDLSVKL CGTISSEISD YFKDDEVYAE YISSYLDFEY
1001 DGGNYKDSLN RFCNSDAVND QKVALYYDGE HPKLNRNIIL
1041 SKLYGERRFL EKITDRVSRS DIVEYYKLKK ETSQYQTKGI
1081 FDSEDEQKNI KKFQEMKNIV EFRDLMDYSE IADELQGQLI
1121 NWIYLRERDL MNFQLGYHYA CLNNDSNKQA TYVTLDYQGK
1161 KNRKINGAIL YQICAMYING LPLYYVDKDS SEWTVSDGKE
1201 STGAKIGEFY RYAKSFENTS DCYASGLEIF ENISEHDNIT
1241 ELRNYIEHER YYSSFDRSEL GIYSEVEDRF FTYDLKYRKN
1281 VPTILYNILL QHFVNVRFEF VSGKKMIGID KKDRKIAKEK
1321 ECARITIREK NGVYSEQFTY KLKNGTVYVD ARDKRYLQSI
1361 IRLLFYPEKV NMDEMIEVKE KKKPSDNNTG KGYSKRDRQQ
1401 DRKEYDKYKE KKKKEGNFLS GMGGNINWDE INAQLKN
```

For example, a Leptotrichia shahii Cas13a endonuclease can be used that has the following amino acid sequence (SEQ ID NO: 44; NCBI accession no. BBM39911.1).

```
      1 MGNLFGHKRW YEVRDKKDFK IKRKVKVKRN YDGNKYILNI
    41 NENNNKEKID NNKFIRKYIN YKKNDNILKE FTRKFHAGNI
    81 LFKLKGKEGI IRIENNDDEL ETEEVVLYIE AYGKSEKLKA
  121 LGITKKKIID EAIRQGITKD DKKIEIKRQE NEEEIEIDIR
  161 DEYTNKTLND CSIILRIIEN DELETKKSIY EIFKNINMSL
  201 YKIIEKIIEN ETEKVFENRY YEEHLREKLL KDDKIDVILT
  241 NFMEIREKIK SNLEILGFVK FYLNVGGDKK KSKNKKMLVE
  281 KILNINVDLT VEDIADFVIK ELEFWNITKR IEKVKKVNNE
  321 FLEKRRNRTY IKSYVLLDKH EKFKIERENK KDKIVKFFVE
  361 NIKNNSIKEK IEKILAEFKI DELIKKLEKE LKKGNCDTEI
  401 FGIFKKHYKV NEDSKKESKK SDEEKELYKI IYRYLKGRIE
  441 KILVNEQKVR LKKMEKIEIE KILNESILSE KILKRVKQYT
  481 LEHIMYLGKL RHNDIDMTTV NTDDFSRLHA KEELDLELIT
  521 FFASTNMELN KIFSRENINN DENIDFFGGD REKNYVLDKK
  561 ILNSKIKIIR DLDFIDNKNN ITNNFIRKFT KIGTNERNRI
  601 LHAISKERDL QGTQDDYNKV INIIQNLKIS DEEVSKALNL
  641 DVVFKDKKNI ITKINDIKIS EENNNDIKYL PSFSKVLPEI
  681 LNLYRNNPKN EPFDTIETEK IVLNALIYVN KELYKKLILE
  721 DDLEENESKN IFLQELKKTL GNIDEIDENI IENYYKNAQI
  761 SASKGNNKAI KKYQKKVIEC YIGYLRKNYE ELFDFSDFKM
  801 NIQEIKKQIK DINDNKTYER ITVKTSDKTI VINDDFEYII
  841 SIFALLNSNA VINKIRNRFF ATSVWLNTSE YQNIIDILDE
  881 IMQLNTLRNE CITENWNLNL EEFIQKMKEI EKDFDDFKIQ
  921 TKKEIFNNYY EDIKNNILTE FKDDINGCDV LEKKLEKIVI
  961 FDDETKFEID KKSNILQDEQ RKLSNINKKD LKKKVDQYIK
1001 DKDQEIKSKI LCRIIFNSDF LKKYKKEIDN LIEDMESENE
1041 NKFQEIYYPK ERKNELYIYK KNLFLNIGNP NEDKIYGLIS
1081 NDIKMADAKF LENIDGKNIR KNKISEIDAI LKNLNDKLNG
1121 YSKEYKEKYI KKLKENDDFF AKNIQNKNYK SFEKDYNRVS
1161 EYKKIRDLVE FNYLNKIESY LIDINWKLAI QMARFERDMH
1201 YIVNGLRELG IIKLSGYNTG ISRAYPKRNG SDGFYTTTAY
1241 YKFFDEESYK KFEKICYGFG IDLSENSEIN KPENESIRNY
1281 ISHFYIVRNP FADYSIAEQI DRVSNLLSYS TRYNNSTYAS
1321 VFEVFKKDVN LDYDELKKKF KLIGNNDILE RLMKPKKVSV
1361 LELESYNSDY IKNLIIELLT KIENTNDTL
```

In another example, a Leptotrichia buccalis C-1013-b Cas13a endonuclease can have the following amino acid sequence (SEQ ID NO: 45; NCBI accession no. C7NBY4; AltName LbuC2c2).

```
      1 MKVTKVGGIS HKKYTSEGRL VKSESEENRT DERLSALLNM
    41 RLDMYIKNPS STETKENQKR IGKLKKFFSN KMVYLKDNTL
    81 SLKNGKKENI DREYSETDIL ESDVRDKKNF AVLKKIYLNE
  121 NVNSEELEVF RNDIKKKLNK INSLKYSFEK NKANYQKINE
  161 NNIEKVEGKS KRNIIYDYYR ESAKRDAYVS NVKEAFDKLY
  201 KEEDIAKLVL EIENLTKLEK YKIREFYHEI IGRKNDKENE
  241 AKIIYEEIQN VNNMKELIEK VPDMSELKKS QVFYKYYLDK
  281 EELNDKNIKY AFCHFVEIEM SQLLKNYVYK RLSNISNDKI
  321 KRIFEYQNLK KLIENKLINK LDTYVRNCGK YNYYLQDGEI
  361 ATSDFIARNR QNEAFLRNII GVSSVAYFSL RNILETENEN
  401 DITGRMRGKT VKNNKGEEKY VSGEVDKIYN ENKKNEVKEN
  441 LKMFYSYDEN MDNKNEIEDF FANIDEAISS IRHGIVHENL
  481 ELEGKDIFAF KNIAPSEISK KMFQNEINEK KLKLKIFRQL
  521 NSANVERYLE KYKILNYLKR TRFEFVNKNI PFVPSFTKLY
  561 SRIDDLKNSL GIYWKTPKTN DDNKTKEIID AQIYLLKNIY
  601 YGEFLNYFMS NNGNFFEISK EIIELNKNDK RNLKTGFYKL
  641 QKFEDIQEKI PKEYLANIQS LYMINAGNQD EEEKDTYIDE
  681 IQKIFLKGFM TYLANNGRLS LIYIGSDEET NTSLAEKKQE
  721 FDKELKKYEQ NNNIKIPYEI NEFLREIKLG NILKYTERLN
  761 MFYLILKLLN HKELTNLKGS LEKYQSANKE EAFSDQLELI
  801 NLLNLDNNRV TEDFELEADE IGKFLDENGN KVKDNKELKK
  841 FDTNKIYFDG ENIIKHRAFY NIKKYGMLNL LEKIADKAGY
  881 KISIEELKKY SNKKNEIEKN HKMQENLHRK YARPRKDEKF
  921 TDEDYESYKQ AIENIEEYTH LKNKVEFNEL NLLQGLLLRI
  961 LHRLVGYTSI WERDLRFRLK GEFPENQYIE EIFNFENKKN
1001 VKYKGGQIVE KYIKFYKELH QNDEVKINKY SSANIKVLKQ
1041 EKKDLYIRNY IAHFNYIPHA EISLLEVLEN LRKLLSYDRK
1081 LKNAVMKSVV DILKEYGFVA TFKIGADKKI GIQTLESEKI
1121VHLKNLKKKK LMTDRNSEEL CKLVKIMFEY KMEEKKSEN
```

The inventors have evaluated the kinetics of other Cas13a and Cas13b proteins. Such work indicates that in some cases Cas13b works faster in a target viral RNA detection assay than Cas13a.

For example, a Cas13b from Prevotella buccae can be used in the Influenza RNA detection methods, compositions and devices. An amino acid sequence for a Prevotella buccae Cas13b protein (NCBI accession no. WP_004343973.1) is shown below as SEQ ID NO:46.

```
      1 MQKQDKLFVD RKKNAIFAFP KYITIMENKE KPEPIYYELT
    41 DKHFWAAFLN LARHNVYTTI NHINRRLEIA ELKDDGYMMG
    81 IKGSWNEQAK KLDKKVRLRD LIMKHFPFLE AAAYEMTNSK
  121 SPNNKEQREK EQSEALSLNN LKNVLFIFLE KLQVLRNYYS
  161 HYKYSEESPK PIFETSLLKN MYKVEDANVR LVKRDYMHHE
  201 NIDMQRDFTH LNRKKQVGRT KNIIDSPNFH YHFADKEGNM
  241 TIAGLLFFVS LFLDKKDAIW MQKKLKGFKD GRNLREQMTN
  281 EVFCRSRISL PKLKLENVQT KDWMQLDMLN ELVRCPKSLY
  321 ERLREKDRES FKVPFDIFSD DYNAEEEPFK NTLVRHQDRE
  361 PYFVLRYFDL NEIFEQLRFQ IDLGTYHFSI YNKRIGDEDE
  401 VRHLTHHLYG FARIQDFAPQ NQPEEWRKLV KDLDHFETSQ
  441 EPYISKTAPH YHLENEKIGI KFCSAHNNLF PSLQTDKTCN
  481 GRSKENLGTQ FTAEAFLSVH ELLPMMFYYL LLTKDYSRKE
  521 SADKVEGIIR KEISNIYAIY DAFANNEINS IADLTRRLQN
  561 TNILQGHLPK QMISILKGRQ KDMGKEAERK IGEMIDDTQR
  601 RLDLLCKQTN QKIRIGKRNA GLLKSGKIAD WLVNDMMRFQ
  641 PVQKDQNNIP INNSKANSTE YRMLQRALAL FGSENFRLKA
  681 YFNQMNLVGN DNPHPFLAET QWEHQTNILS FYRNYLEARK
  721 KYLKGLKPQN WKQYQHFLIL KVQKTNRNTL VTGWKNSENL
  761 PRGIFTQPIR EWFEKHNNSK RIYDQILSFD RVGFVAKAIP
  801 LYFAEEYKDN VQPFYDYPEN IGNRLKPKKR QFLDKKERVE
  841 LWQKNKELFK NYPSEKKKTD LAYLDFLSWK KFERELRLIK
  881 NQDIVTWLMF KELENMATVE GLKIGEIHLR DIDTNTANEE
  921 SNNILNRIMP MKLPVKTYET DNKGNILKER PLATFYIEET
  961 ETKVLKQGNF KALVKDRRLN GLFSFAETTD LNLEEHPISK
1001 LSVDLELIKY QTTRISIFEM TLGLEKKLID KYSTLPTDSF
1041 RNMLERWLQC KANRPELKNY VNSLIAVRNA FSHNQYPMYD
1081 ATLFAEVKKF TLFPSVDTKK IELNIAPQLL EIVGKAIKEI
1121 EKSENKN
```

Such a Prevotella buccae Cas13b protein can have a Km (Michaelis constant) substrate concentration of about 20 micromoles and a Kcat of about 987/second (see, e.g., Slaymaker et al. Cell Rep 26 (13): 3741-3751 (2019)).

Another Prevotella buccae Cas13b protein (NCBI accession no. WP_004343581.1) that can be used in the SARS-CoV-2 RNA detection methods, compositions and devices has the amino acid sequence shown below as SEQ ID NO: 47.

```
      1 MQKQDKLFVD RKKNAIFAFP KYITIMENQE KPEPIYYELT
    41 DKHFWAAFLN LARHNVYTTI NHINRRLEIA ELKDDGYMMD
    81 IKGSWNEQAK KLDKKVRLRD LIMKHEPFLE AAAYEITNSK
  121 SPNNKEQREK EQSEALSLNN LKNVLFIFLE KLQVLRNYYS
  161 HYKYSEESPK PIFETSLLKN MYKVEDANVR LVKRDYMHHE
  201 NIDMQRDFTH LNRKKQVGRT KNIIDSPNFH YHFADKEGNM
  241 TIAGLLFFVS LFLDKKDAIW MQKKLKGFKD GRNLREQMTN
  281 EVFCRSRISL PKLKLENVQT KDWMQLDMLN ELVRCPKSLY
  321 ERLREKDRES FKVPFDIFSD DYDAEEEPFK NTLVRHQDRE
  361 PYFVLRYFDL NEIFEQLRFQ IDLGTYHFSI YNKRIGDEDE
  401 VRHLTHHLYG FARIQDFAQQ NQPEVWRKLV KDLDYFEASQ
  441 EPYIPKTAPH YHLENEKIGI KFCSTHNNLF PSLKTEKTCN
  481 GRSKENLGTQ FTAEAFLSVH ELLPMMFYYL LLTKDYSRKE
  521 SADKVEGIIR KEISNIYAIY DAFANGEINS IADLTCRLQK
  561 TNILQGHLPK QMISILEGRQ KDMEKEAERK IGEMIDDTQR
  601 RLDLLCKQTN QKIRIGKRNA GLLKSGKIAD WLVNDMMRFQ
  641 PVQKDQNNIP INNSKANSTE YRMLQRALAL FGSENFRLKA
  681 YFNQMNLVGN DNPHPFLAET QWEHQTNILS FYRNYLEARK
  721 KYLKGLKPQN WKQYQHFLIL KVQKINRNTL VTGWKNSENL
  761 PRGIFTQPIR EWFEKHNNSK RIYDQILSFD RVGFVAKAIP
  801 LYFAEEYKDN VQPFYDYPFN IGNKLKPQKG QFLDKKERVE
  841 LWQKNKELFK NYPSEKKKTD LAYLDELSWK KFERELRLIK
  881 NQDIVTWLMF KELENMATVE GLKIGEIHLR DIDTNTANEE
  921 SNNILNRIMP MKLPVKTYET DNKGNILKER PLATFYIEET
  961 ETKVLKQGNF KVLAKDRRLN GLLSFAETTD IDLEKNPITK
1001 LSVDHELIKY QTTRISIFEM TLGLEKKLIN KYPTLPTDSF
1041 RNMLERWLQC KANRPELKNY VNSLIAVRNA FSHNQYPMYD
1081 ATLFAEVKKF TLFPSVDTKK IELNIAPQLL EIVGKAIKEI
1121 EKSENKN
```

An example of a Bergeyella zoohelcum Cas13b (R1177A) mutant amino acid sequence (NCBI accession no. 6AAY_A) is shown below as SEQ ID NO: 48.

```
      1 XENKTSLGNN IYYNPFKPQD KSYFAGYFNA AXENTDSVER
    41 ELGKRLKGKE YTSENFFDAI FKENISLVEY ERYVKLLSDY
    81 FPXARLLDKK EVPIKERKEN FKKNFKGIIK AVRDLRNFYT
  121 HKEHGEVEIT DEIFGVLDEX LKSTVLTVKK KKVKTDKTKE
  161 ILKKSIEKQL DILCQKKLEY LRDTARKIEE KRRNQRERGE
  201 KELVAPFKYS DKRDDLIAAI YNDAFDVYID KKKDSLKESS
  241 KAKYNTKSDP QQEEGDLKIP ISKNGVVFLL SLFLTKQEIH
  281 AFKSKIAGFK ATVIDEATVS EATVSHGKNS ICFXATHEIF
  321 SHLAYKKLKR KVRTAEINYG EAENAEQLSV YAKETLXXQX
  361 LDELSKVPDV VYQNLSEDVQ KTFIEDWNEY LKENNGDVGT
  401 XEEEQVIHPV IRKRYEDKEN YFAIRFLDEF AQFPTLRFQV
  441 HLGNYLHDSR PKENLISDRR IKEKITVEGR LSELEHKKAL
  481 FIKNTETNED REHYWEIFPN PNYDFPKENI SVNDKDEPIA
  521 GSILDREKQP VAGKIGIKVK LLNQQYVSEV DKAVKAHQLK
  561 QRKASKPSIQ NIIEEIVPIN ESNPKEAIVF GGQPTAYLSX
  601 NDIHSILYEF FDKWEKKKEK LEKKGEKELR KEIGKELEKK
  641 IVGKIQAQIQ QIIDKDTNAK ILKPYQDGNS TAIDKEKLIK
  681 DLKQEQNILQ KLKDEQTVRE KEYNDFIAYQ DKNREINKVR
  721 DRNHKQYLKD NLKRKYPEAP ARKEVLYYRE KGKVAVWLAN
  761 DIKRFXPTDF KNEWKGEQHS LLQKSLAYYE QCKEELKNLL
  801 PEKVFQHLPF KLGGYFQQKY LYQFYTCYLD KRLEYISGLV
  841 QQAENFKSEN KVFKKVENEC FKFLKKQNYT HKELDARVQS
  881 ILGYPIFLER GFXDEKPTII KGKTFKGNEA LFADWFRYYK
  921 EYQNFQTFYD TENYPLVELE KKQADRKRKT KIYQQKKNDV
  961 FTLLXAKHIF KSVFKQDSID QFSLEDLYQS REERLGNQER
1001 ARQTGERNTN YIWNKTVDLK LCDGKITVEN VKLKNVGDFI
1041 KYEYDQRVQA FLKYEENIEW QAFLIKESKE EENYPYVVER
1081 EIEQYEKVRR EELLKEVHLI EEYILEKVKD KEILKKGDNQ
1121 NFKYYILNGL LKQLKNEDVE SYKVENLNTE PEDVNINQLK
1161 QEATDLEQKA FVLTYIANKE AHNQLPKKEF WDYCQEKYGK
1201 IEKEKTYAEY FAEVEKKEKE ALIKLEHHHH HH
```

Another example of a Cas13b protein sequence from Prevotella sp. MSX73 (NCBI accession no. WP_007412163.1) that can be used in the target viral RNA detection methods, compositions and devices is shown below as SEQ ID NO: 49.

```
      1 MQKQDKLFVD RKKNAIFAFP KYITIMENQE KPEPIYYELT
    41 DKHFWAAFLN LARHNVYTTI NHINRRLEIA ELKDDGYMMG
    81 IKGSWNEQAK KLDKKVRLRD LIMKHFPFLE AAAYEITNSK
  121 SPNNKEQREK EQSEALSLNN LKNVLFIFLE KLQVLRNYYS
  161 HYKYSEESPK PIFETSLLKN MYKVEDANVR LVKRDYMHHE
  201 NIDMQRDFTH LNRKKQVGRT KNIIDSPNFH YHFADKEGNM
  241 TIAGLLFFVS LFLDKKDAIW MQKKLKGFKD GRNLREQMTN
  281 EVFCRSRISL PKLKLENVQT KDWMQLDMLN ELVRCPKSLY
  321 ERLREKDRES FKVPFDIFSD DYDAEEEPFK NTLVRHQDRE
  361 PYFVLRYFDL NEIFEQLRFQ IDLGTYHFSI YNKRIGDEDE
  401 VRHLTHHLYG FARIQDFAPQ NQPEEWRKLV KDLDHFETSQ
  441 EPYISKTAPH YHLENEKIGI KFCSTHNNLF PSLKREKTCN
  481 GRSKENLGTQ FTAEAFLSVH ELLPMMFYYL LLTKDYSRKE
  521 SADKVEGIIR KEISNIYAIY DAFANNEINS IADLTCRLQK
  561 TNILQGHLPK QMISILEGRQ KDMEKEAERK IGEMIDDTQR
  601 RLDLLCKQTN QKIRIGKRNA GLLKSGKIAD WLVSDMMRFQ
  641 PVQKDTNNAP INNSKANSTE YRMLQHALAL FGSESSRLKA
  681 YFRQMNLVGN ANPHPFLAET QWEHQTNILS FYRNYLEARK
  721 KYLKGLKPQN WKQYQHFLIL KVQKTNRNTL VTGWKNSFNL
  761 PRGIFTQPIR EWFEKHNNSK RIYDQILSFD RVGFVAKAIP
  801 LYFAEEYKDN VQPFYDYPFN IGNKLKPQKG QFLDKKERVE
  841 LWQKNKELFK NYPSEKNKTD LAYLDELSWK KFERELRLIK
  881 NQDIVTWLMF KELFKTTTVE GLKIGEIHLR DIDTNTANEE
  921 SNNILNRIMP MKLPVKTYET DNKGNILKER PLATFYIEET
  961 ETKVLKQGNF KVLAKDRRLN GLLSFAETTD IDLEKNPITK
1001 LSVDYELIKY QTTRISIFEM TLGLEKKLID KYSTLPTDSF
1041 RNMLERWLQC KANRPELKNY VNSLIAVRNA FSHNQYPMYD
1081 ATLFAEVKKF TLFPSVDTKK IELNIAPQLL EIVGKAIKEI
1121 EKSENKN
```

Hence, the sample can be incubated with at least one CRISPR RNA (crRNA) and at least one Cas13 protein. The Cas13 protein can, for example, be a Cas13a protein, Cas13b protein, or a combination thereof.

### (CRISPR)/CRISPR-Associated (Cas) Systems

Genomic editing has been performed by using clustered regularly interspaced short palindromic repeats (CRISPR)/CRISPR-associated (Cas) systems (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties).

However, a CRISPR guide RNA system can be adapted for use in the methods and compositions described herein. Two RNAs can be used in CRISPR genomic editing systems: a CRISPR RNA (crRNA), which is a 17-20 nucleotide sequence complementary to the target RNA, and a trans-activating crRNA (tracrRNA) that is a binding scaffold for the Cas nuclease. In some cases, the two RNAs are fused to make a single guide RNA (sgRNA). The tracrRNA forms a stem loop that is recognized and bound by the cas nuclease. The crRNA typically has shorter sequence than the tracrRNA. The term “guide RNA” as used herein refers to either a single guide RNA (sgRNA) or a crRNA. The CRISPR technique is generally described, for example, by Mali et al. Science 339:823-6 (2013); which is incorporated by reference herein in its entirety.

The guide RNA system used herein is encoded within or adjacent to the ncRNA coding region of the expression cassettes. Hence, upon transcription of the guide RNA, it can target a Cas enzyme to the desired location in the genome, where it can cleave the genomic RNA for generation of a genomic modification.

There are several types of CRISPR systems, some of which are summarized in the chart below.

### CRISPR System Types Overview

| System | Features | Examples |
|---|---|---|
| Type I | Multiple proteins (5-7 proteins | Staphylococcus epidermidis |
| | typical), crRNA. DNA Cleavage | (Type IA) |
| | is catalyzed by Cas3. | |
| Type II | 3-4 proteins (one protein (Cas9) | Streptococcus pyogenes |
| | has nuclease activity) two RNAs. | CRISPR/Cas9, Francisella |
| | Target DNA cleavage catalyzed by | novicida U112 Cpf1 |
| | Cas9 and RNA components. | |
| Type III | Five or six proteins required for | S. epidermidis (Type IIIA); |
| | cutting, number of required RNAs | P. furiosus; (Type IIIB). |
| | unknown but expected to be one. | |
| | Type IIIB systems have the ability | |
| | to target RNA. | |

A “guide RNA” or “gRNA” as provided herein refers to a ribonucleotide sequence capable of binding a cas nuclease, thereby forming ribonucleoprotein complex. The gRNA includes a nucleotide sequence complementary to a target site (e.g., near or at a genomic site to be edited). In some cases, the guide RNA includes one or more RNA molecules. TracrRNAs can be used to facilitate assembly of a ribonucleoprotein complex that includes the gRNA together with the tracrRNA and a cas nuclease. A complementary nucleotide sequence of the guide RNA can mediate binding of the ribonucleoprotein complex to the target site thereby providing the sequence specificity of the ribonucleoprotein complex. Thus, the guide RNA includes a sequence that is complementary to a target nucleic acid sequence such that the guide RNA binds a target nucleic acid sequence.

In some cases, the complement of the guide RNA includes a sequence having a sequence identity of about 50%, 55%, 60%, 65%, 70%, 75%, 80%, 85%, 90%, 95%, 96%, 97%, 98%, 99%, or 100% to a target nucleic acid (e.g., a target viral RNA sequence). In some cases, the guide RNA includes a sequence having sequence identity of about 50%, 55%, 60%, 65%, 70%, 75%, 80%, 85%, 90%, 95%, 96%, 97%, 98%, 99%, or 100% to the target nucleic acid sequence. In some cases, the guide RNA or complement thereof, includes a sequence having a sequence identity of at least about 90%, 95%, or 100% to a target viral RNA sequence. In some cases, segment bound by a guide RNA within the target nucleic acid is about or at least about 10, 15, 20, 25, or more nucleotides in length.

The guide RNA is a single-stranded ribonucleic acid, although in some cases it may form some double-stranded regions by folding onto itself. In some cases, the guide RNA is about 10, 20, 30, 40, 50, 60, 70, 80, 90, 100 or more nucleic acid residues in length. In some cases, the guide RNA is from about 10 to about 30 nucleic acid residues in length. In some cases, the guide RNA is about 20 nucleic acid residues in length. For example, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides or residues in length. In some cases, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more nucleotides or residues in length. In some cases, the guide RNA is from 10 to 15, 10 to 20, 10 to 30, 10 to 40, or 10 to 50 residues in length.

### Definitions

The term “about” as used herein when referring to a measurable value such as an amount, a length, and the like, is meant to encompass variations of ±20% or ±10%, more preferably ±5%, even more preferably ±1%, and still more preferably ±0.1% from the specified value.

“Recombinant” as used herein to describe a nucleic acid molecule means a polynucleotide of genomic, cDNA, bacterial, viral, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed organisms, for example, as described herein. The host organism expresses the foreign nucleic acids to produce the RNA, RT-DNA, or protein under expression conditions.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide or a selected RNA, is a nucleic acid molecule which is transcribed (in the case of DNA templates) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, ncRNAs, tracrRNAs, ncRNAs modified to include heterologous sequences, cDNA from viral, prokaryotic or eukaryotic ncRNA, mRNA, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper polymerases are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein, DNA, or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Mammalian cell” refers to any cell derived from a mammalian subject suitable for transfection with vector systems comprising, as described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “donor polynucleotide” or “donor DNA” refers to a nucleic acid or polynucleotide that provides a nucleotide sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide (donor DNA). The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system are derived from a particular organism comprising an endogenous CRISPR system, such as Streptococcus pyogenes. In general, a CRISPR system can be characterized by elements that promote the formation of a CRISPR complex at the site of a target sequence (also referred to as a protospacer in the context of an endogenous CRISPR system).

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al., Biochim. Biophys. Acta 1307:26-30 (1996); and Mojica et al., Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al., Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et al., Mol. Microbiol., 43:1565-1575 (2002); and Mojica et al, (2005)) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme (e.g., cas9) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

“Administering” a nucleic acid, such as an expression cassette, comprises transducing, transfecting, electroporating, translocating, fusing, phagocytosing, shooting or ballistic methods, etc., i.e., any means by which a nucleic acid can be transported across a cell membrane.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

The following Examples illustrate some of the materials, methods, and experiments that were used or performed in the development of the invention.

### EXAMPLES

### Example 1: Cas13a Detection of SARS-CoV-2 Transcripts

CRISPR RNA guides (crRNAs) were designed and validated for Influenza A, strains H1N2 and H3N2, and Influenza B. Twenty-two (22) crRNAs were designed for Influenza A and fifteen (15) crRNAs were designed for Influenza A. Each crRNA includes a crRNA stem that is derived from a bacterial sequence, while the spacer sequence is derived from the Influenza genome (reverse complement). See Table 1 (reproduced below) for crRNA sequences.

TABLE 1
Examples of Influenza A and B crRNA Sequences

| SEQ ID NO | Name | Full crRNA (stem + TARGET) |
|---|---|---|
| SEQ ID NO: 1 | FluA_cr01m | gaccaccccaaaaaugaaggggacuaaaac GAGCCGAACGGCUGCAUUGA |
| SEQ ID NO: 2 | FluA_cr02m | gaccaccccaaaaaugaaggggacuaaaac CUGGACAGUGGUGAACAGUA |
| SEQ ID NO: 3 | FluA_cr03m | gaccaccccaaaaaugaaggggacuaaaac CCUGAAGUCUGCUUAAAGUG |
| SEQ ID NO: 4 | FluA_cr04m | gaccaccccaaaaaugaaggggacuaaaac AAUUUUUCCCUAGUAGUUCA |
| SEQ ID NO: 5 | FluA_cr05m | gaccaccccaaaaaugaaggggacuaaaac CCCUAGUAGUUCAUAUAGGA |
| SEQ ID NO: 6 | FluA_cr06m | gaccaccccaaaaaugaaggggacuaaaac GUGCCAUAGAGGAGACACAC |
| SEQ ID NO: 7 | FluA_cr07 | gaccaccccaaaaaugaaggggacuaaaac UAUGGCCAUAAUUAAGAAGU |
| SEQ ID NO: 8 | FluA_cr08 | gaccaccccaaaaaugaaggggacuaaaac AAUUUUUCCCUAGUAGUUCA |
| SEQ ID NO: 9 | FluA_cr09 | gaccaccccaaaaaugaaggggacuaaaac UCAGCUGACAUGAGUAUUGG |
| SEQ ID NO: 10 | FluA_cr10 | gaccaccccaaaaaugaaggggacuaaaac CAGCUGACAUGAGUAUUGGA |
| SEQ ID NO: 11 | FluA_cr11 | gaccaccccaaaaaugaaggggacuaaaac GACAUGAGUAUUGGAGUAAC |
| SEQ ID NO: 12 | FluA_cr12 | gaccaccccaaaaaugaaggggacuaaaac CCUGAAGUCUGCUUAAAGUG |
| SEQ ID NO: 13 | FluA_cr13 | gaccaccccaaaaaugaaggggacuaaaac GAGUCAGGAAGGCUAAUAGA |
| SEQ ID NO: 14 | FluA_cr14 | gaccaccccaaaaaugaaggggacuaaaac CGGUUGGAAUUUCUAGCAUG |
| SEQ ID NO: 15 | FluA_cr15 | gaccaccccaaaaaugaaggggacuaaaac GGUUGGAAUUUCUAGCAUGG |
| SEQ ID NO: 16 | FluA_cr16 | gaccaccccaaaaaugaaggggacuaaaac AUAUGAAGCAAUCGAGGAGU |
| SEQ ID NO: 17 | FluA_cr17 | gaccaccccaaaaaugaaggggacuaaaac UAUGAAGCAAUCGAGGAGUG |
| SEQ ID NO: 18 | FluA_cr18 | gaccaccccaaaaaugaaggggacuaaaac CCUGCCUGCUUGUGUGUAUG |
| SEQ ID NO: 19 | FluA_cr19 | gaccaccccaaaaaugaaggggacuaaaac CGGCAAUGGUGUUUGGAUAG |
| SEQ ID NO: 20 | FluA_cr20 | gaccaccccaaaaaugaaggggacuaaaac AACGUACGUUCUUUCUAUCA |
| SEQ ID NO: 21 | FluA_cr21 | gaccaccccaaaaaugaaggggacuaaaac UAUACAGAGAUUCGCUUGGA |
| SEQ ID NO: 22 | FluA_cr22 | gaccaccccaaaaaugaaggggacuaaaac AUACAGAGAUUCGCUUGGAG |
| SEQ ID NO: 23 | FluB_cr01 | gaccaccccaaaaaugaaggggacuaaaac UACUACAAAAAUCCCGGAAC |
| SEQ ID NO: 24 | FluB_cr02 | gaccaccccaaaaaugaaggggacuaaaac AGAUAGCAUAUUAAACAUUC |
| SEQ ID NO: 25 | FluB_cr03 | gaccaccccaaaaaugaaggggacuaaaac AUUUAUCCCAUGUAGCUCAC |
| SEQ ID NO: 26 | FluB_cr04 | gaccaccccaaaaaugaaggggacuaaaac GACAUCAUUCUGCCGCAUAU |
| SEQ ID NO: 27 | FluB_cr05 | gaccaccccaaaaaugaaggggacuaaaac AGACAUCUUCUAGCUUCCAU |
| SEQ ID NO: 28 | FluB_cr06 | gaccaccccaaaaaugaaggggacuaaaac UCCCAGUGCAGAUUCGAUCU |
| SEQ ID NO: 29 | FluB_cr07 | gaccaccccaaaaaugaaggggacuaaaac AUCCCAGUGCAGAUUCGAUC |
| SEQ ID NO: 30 | FluB_cr08 | gaccaccccaaaaaugaaggggacuaaaac UAUCCCAGUGCAGAUUCGAU |
| SEQ ID NO: 31 | FluB_cr09 | gaccaccccaaaaaugaaggggacuaaaac UUAUCCCAGUGCAGAUUCGA |
| SEQ ID NO: 32 | FluB_cr10 | gaccaccccaaaaaugaaggggacuaaaac GUCGUGCAUUAUAGGAAAGC |
| SEQ ID NO: 33 | FluB_cr11 | gaccaccccaaaaaugaaggggacuaaaac UUCAUACCCAACCAUAGAGU |
| SEQ ID NO: 34 | FluB_cr12 | gaccaccccaaaaaugaaggggacuaaaac GACAGCAUUCUUCUUACAGC |
| SEQ ID NO: 35 | FluB_cr13 | gaccaccccaaaaaugaaggggacuaaaac GAUAAGACUCCCACCGCAGU |
| SEQ ID NO: 36 | FluB_cr14 | gaccaccccaaaaaugaaggggacuaaaac GCUGUACACUUCAACCACAU |
| SEQ ID NO: 37 | FluB_cr15 | gaccaccccaaaaaugaaggggacuaaaac UGCCUGCUGUACACUUCAAC |

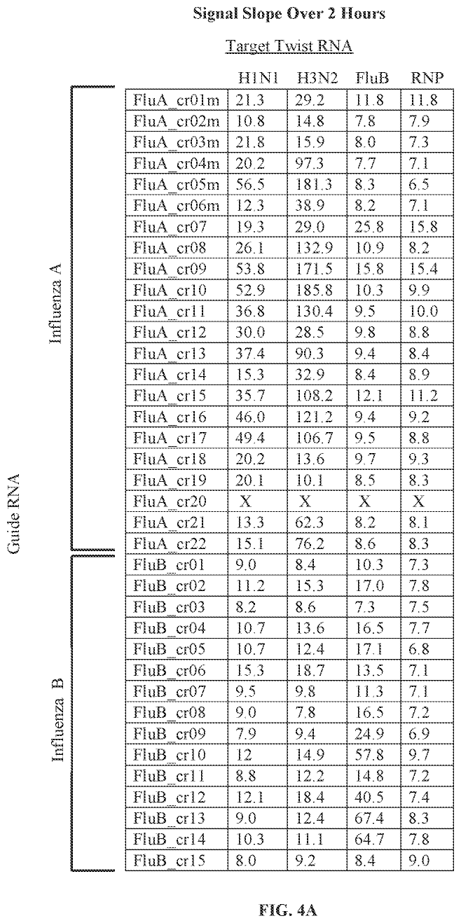

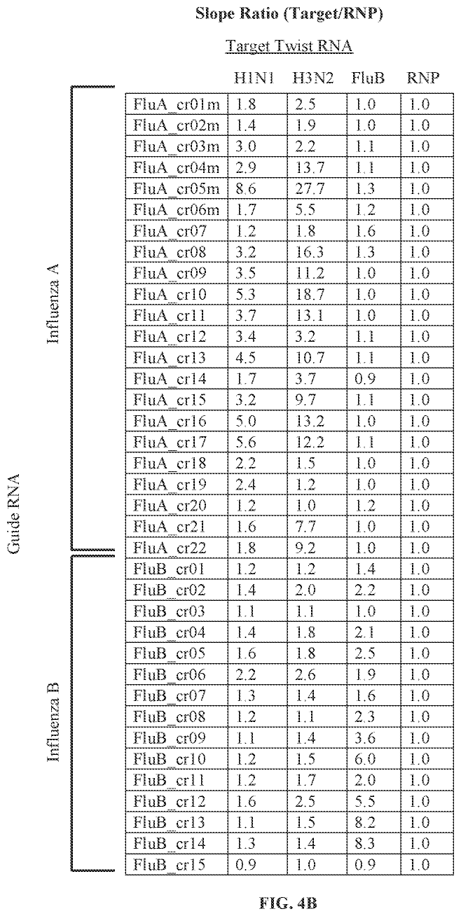

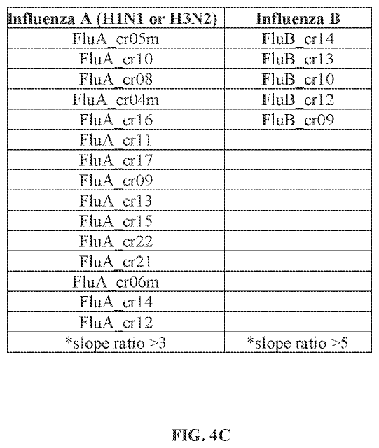

FIGS. 4A-C shows the detection of influenza strains with the specific RNA guides of Table 1. The RNA guides were tested against H1N1, H3N2, FluB target viral RNA, and ribonucleoprotein (RNP) background control with no target viral RNA. As shown in FIG. 4A, the signals from each reaction were measured over two hours and the signal slopes were calculated. Slope ratios were calculated by dividing the slope of a guide RNA+target (i.e. RNP+target viral RNA) reaction by the slope of guide RNA+no target (i.e. RNP control only) reaction. As shown in FIG. 4B, the signal slopes of H1N1, H3N2, or FluB RNA guides was divided by the signal slopes of the RNP control determine comparative slope ratio between the target viral RNA and the RNP control. When the comparative ratio is high (greater than 1), the guide RNAs employed in the assay mixture detect H1N1, H3N2, or FluB target viral RNA strains more efficiently. But when the comparative ratio is low (less than 1), the guide RNAs employed in the assay mixture detect the target viral RNA similarly to the RNP control. FIG. 4C shows the RNA guides for H1N1 and H3N2 strains of Influenza A with a slope ratio of more than three and the RNA guides for FluB with a slope ratio of more than five.

### Example 2: Cas13a Detection of Influenza B RNA in Nasal Swabs

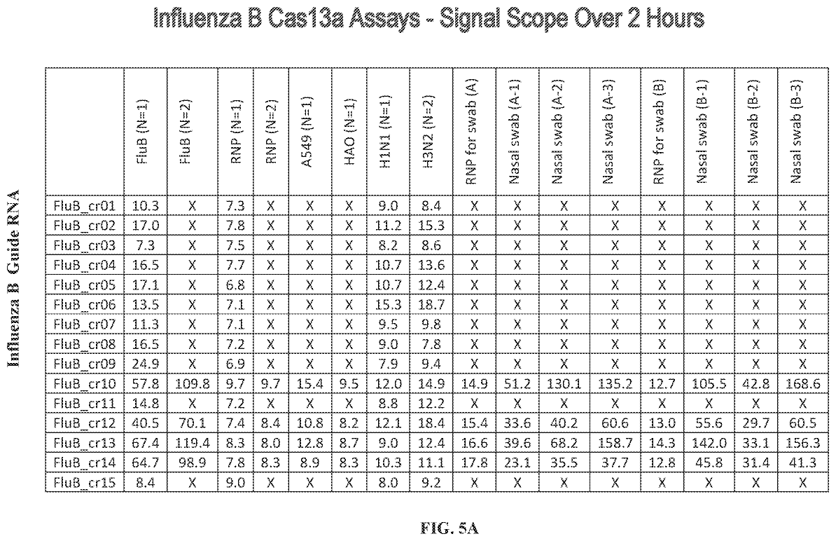

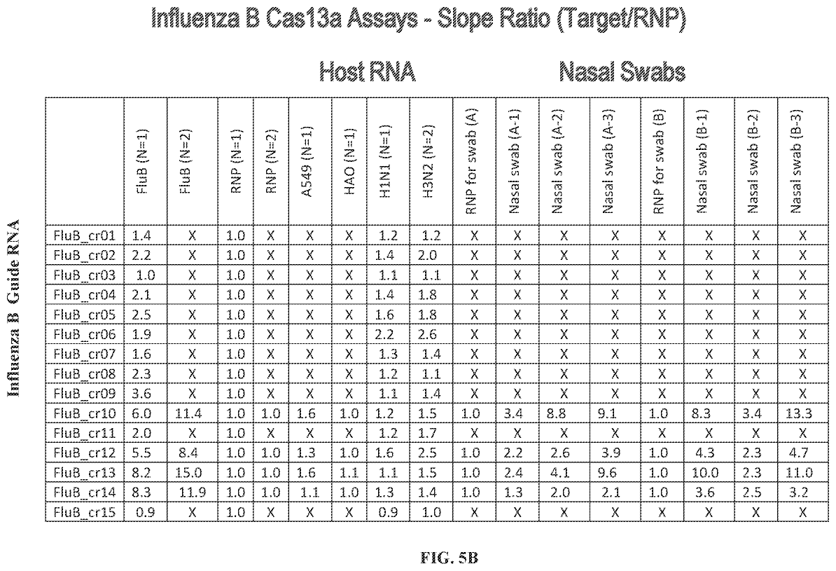

FIGS. 5A-B show the validation and cross-reactivity of Influenza B (FluB) RNA guides against host RNA and nasal swabs. The RNA guides for FluB having a slope ratio of more than five, as shown in FIG. 4C, were tested against host RNA and nasal swabs. The signals from each reaction were measured over two hours and the signal slopes were calculated. To prepare the graph shown in FIG. 5B, the signal slopes of the RNA guides for FluB was divided by the signal slopes of the RNP control determine a comparative slope ratio between the target viral RNA and the RNP control. FluB RNA guides FluB_cr10 and FluB-cr13 were found to cross-react significantly with nasal swab material that was positive for FluB. FluB_cr12 and FluB-cr14 were found to not cross-react to the same extent with the nasal swab material that was positive for FluB.

### Example 3: Improving Detection of Influenza B by Combining RNA Guides of SEQ. ID. NOs: 34 and 36

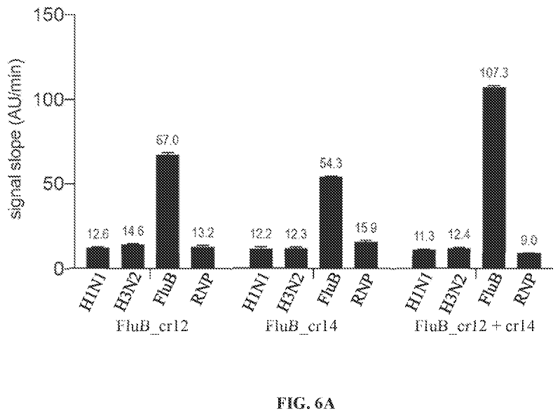

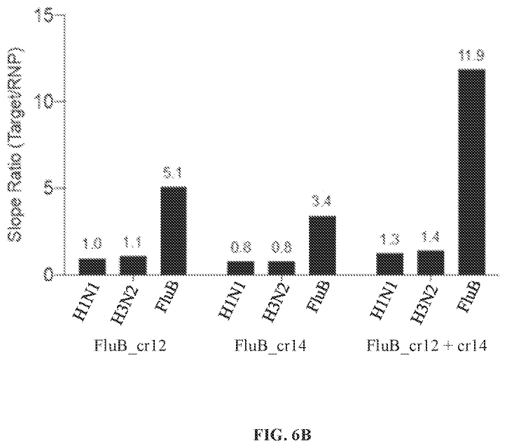

FIGS. 6A-B illustrate the effect on target viral RNA detection of combining the RNA guides FluB_cr12 and FluB-cr14 (SEQ. ID. NOs: 34 and 36). The signals slope from each reaction of target viral RNA for H3N1, H1N1, FluB, or RNP alone with the RNA guides FluB_cr12 alone, FluB-cr14 alone, or FluB_cr12 and FluB-cr14 combined were measured over two hours and the signal slopes were calculated and shown in FIG. 6A. To prepare the graph shown in FIG. 6B, the signal slopes of FIG. 6A were divided by the signal slopes of the RNP control to determine comparative slope ratio between the target viral RNA and the RNP control. Combining the RNA guides FluB_cr12 and FluB-cr14 improves detection of FluB target viral RNA more than use of these RNA guides separately. Detection of H3N1, H1N1, or RNP alone was not increased by combining the RNA guides FluB_cr12 and FluB-cr14.

### Example 4: Validation and Cross-Reactivity of Influenza a (H1N1 and H3N2 Strains) RNA Guides Against Host RNA and Nasal Swabs

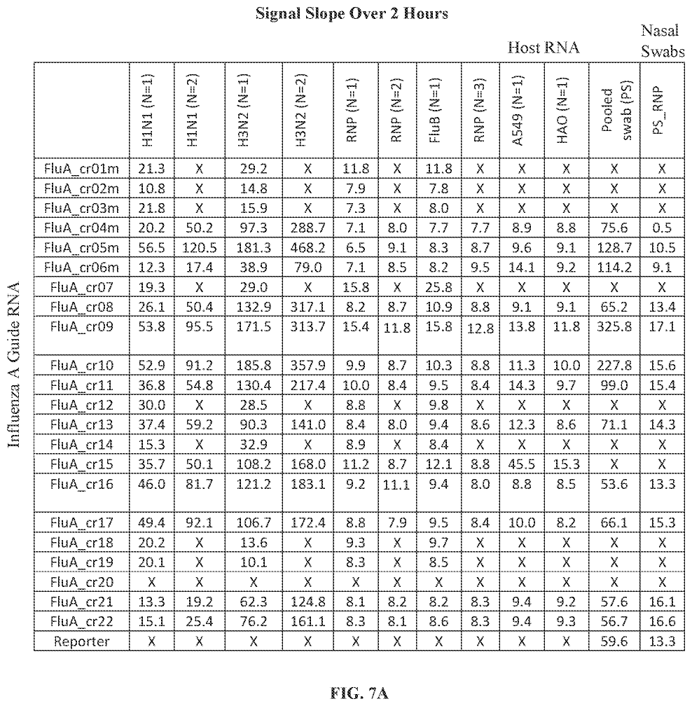

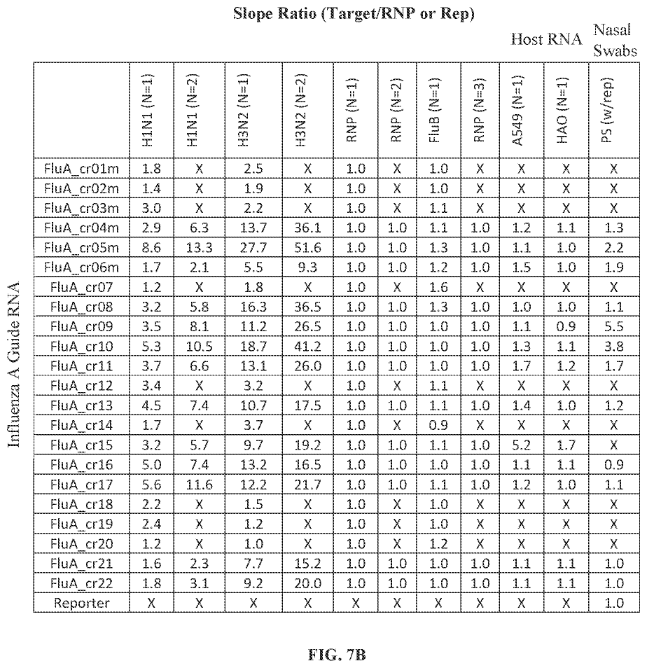

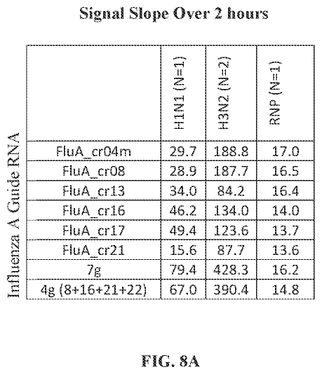

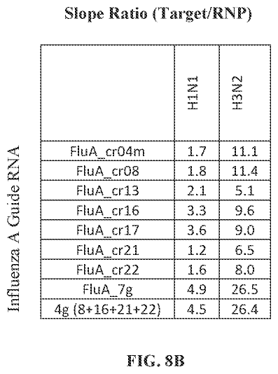

FIGS. 7A-B show the validation and cross-reactivity of Influenza A (H1N1 and H3N2 strains) RNA guides against host RNA and nasal swabs. The signals from each reaction were measured over two hours and the signal slopes were calculated, as shown in FIG. 7A. The RNA guides for Influenza A having a slope ratio of more than three, as shown in FIG. 4C, were included in the test against host RNA and nasal swabs. To prepare the graph shown in FIG. 7B, the signal slopes of the RNA guides for FluB was divided by the signal slopes of the RNP control determine a comparative slope ratio between the target viral RNA and the RNP control. Influenza A RNA guides identified in the boxes had the best detection of the target viral RNA in the nasal swabs and were selected for a combination experiment shown in FIGS. 8A-B.

### Example 5: Improving Detection of Influenza a by Combining RNA Guides of SEQ. ID. NOs: 4, 8, 13, 16, 17, 21, 22 and, Independently, 8, 16, 21, and 22

FIGS. 8A-B shows the effect of combining the best Influenza A RNA guides of FIG. 7A on Influenza A target viral RNA detection. A combination of seven Influenza A RNA guides (the “7 g”: cr04m, cr08, cr13, cr16, cr17, cr21, cr22 (SEQ. ID. NOs: 4, 8, 13, 16, 17, 21, 22, respectively)) and four Influenza RNA guides (the “4 g”: cr08, cr16, cr21, cr22 (SEQ. ID. NOs: 8, 16, 21, and 22, respectively)) were tested against target viral RNA for Influenza A (strains H1N1 and H3N2). The signals from each reaction were measured over two hours and the signal slopes were calculated, as shown in FIG. 8A. To prepare the graph shown in FIG. 8B, the signal slopes of the RNA guides for Influenza A were divided by the signal slopes of the RNP control determine a comparative slope ratio between the target viral RNA and the RNP control. The slope ratios for the Influenza A RNA guides in the 7 g group were 4.9 and 26.5 for the target viral RNA for H1N1 and H3N2, respectively. The slope ratios for the Influenza A RNA guides in the 4 g group were 4.5 and 26.4 for the target viral RNA for H1N1 and H3N2, respectively. The slope ratios for each of the 7 g and 4 g RNA guide groups were significantly higher than for any of the Influenza A RNA guides alone.

### REFERENCES

Abudayyeh O. O., Gootenberg J S, Essletzbichler P, Han S, Joung J, Belanto J J, Verdine V, Cox D B T, Kellner M J, Regev A, Lander E S, Voytas D F, Ting A Y, Zhang F. RNA targeting with CRISPR-Cas13. Nature. Nature Publishing Group; 2017 Oct. 12; 550(7675):280-4.
Abudayyeh, O. O., Gootenberg, J. S., Konermann, S., Joung, J., Slaymaker, I. M., Cox, D. B., Shmakov, S., Makarova, K. S., Semenova, E., Minakhin, L., et al. (2016). C2c2 is a single-component programmable RNA-guided RNA-targeting CRISPR effector. Science. 353(6299): 353, aaf5573.
Babin, S. M., Hsieh, Y. H., Rothman, R. E., and Gaydos, C. A. (2011). A meta-analysis of point-of-care laboratory tests in the diagnosis of novel 2009 swine-lineage pandemic influenza A (H1N1). Diagn Microbiol Infect Dis. 69(4), 410-418.
Chartrand, C., Leeflang, M. M., Minion, J., Brewer, T., and Pai, M. (2012). Accuracy of rapid influenza diagnostic tests: a meta-analysis. Ann Intern Med. 156(7), 500-511. Published online 2012/03/01 DOI: 10.7326/0003-4819-156-7-201204030-00403.
Chen, J. S., Ma, E., Harrington, L. B., Da Costa, M., Tian, X., Palefsky, J. M., and Doudna, J. A. (2018). CRISPR-Cas12a target binding unleashes indiscriminate single-stranded DNase activity. Science. 360(6387), 436-439. Published online 2018/02/17 DOI: 10.1126/science.aar6245.
Chu, H., Lofgren, E. T., Halloran, M. E., Kuan, P. F., Hudgens, M., and Cole, S. R. (2012). Performance of rapid influenza H1N1 diagnostic tests: a meta-analysis. Influenza Other Respir Viruses. 6(2), 80-86. Published online 2011/09/03 DOI: 10.1111/j.1750-2659.2011.00284.x.
East-Seletsky, A., O'Connell, M. R., Burstein, D., Knott, G. J., and Doudna, J. A. (2017). RNA Targeting by Functionally Orthogonal Type VI-A CRISPR-Cas Enzymes. Mol Cell. 66(3), 373-383 e373.
East-Seletsky, A., O'Connell, M. R., Knight, S. C., Burstein, D., Cate, J. H., Tjian, R., and Doudna, J. A. (2016). Two distinct RNase activities of CRISPR-C2c2 enable guide-RNA processing and RNA detection. Nature. 538(7624), 270-273.
Gootenberg, J. S., Abudayyeh, O. O., Kellner, M. J., Joung, J., Collins, J. J., and Zhang, F. (2018). Multiplexed and portable nucleic acid detection platform with Cas13, Cas12a, and Csm6. Science. 360(6387), 439-444.
Gootenberg, J. S., Abudayyeh, O. O., Lee, J. W., Essletzbichler, P., Dy, A. J., Joung, J., Verdine, V., Donghia, N., Daringer, N. M., Freije, C. A., et al. (2017). Nucleic acid detection with CRISPR-Cas13a/C2c2. Science. 356(6336), 438-442.
Green, D. A., and StGeorge, K. (2018). Rapid Antigen Tests for Influenza: Rationale and Significance of the FDA Reclassification. J Clin Microbiol. 56(10). Published online 2018/06/15 DOI: 10.1128/JCM.00711-18.
Gu W, Crawford E D, O'Donovan B D, Wilson M R, Chow E D, Retallack H, DeRisi J L. Depletion of Abundant Sequences by Hybridization (DASH): using Cas9 to remove unwanted high-abundance species in sequencing libraries and molecular counting applications. Genome Biol. BioMed Central; 2016 Mar. 4; 17(1):41.
Myhrvold, C., Freije, C. A., Gootenberg, J. S., Abudayyeh, O. O., Metsky, H. C., Durbin, A. F., Kellner, M. J., Tan, A. L., Paul, L. M., Parham, L. A., et al. (2018). Field-deployable viral diagnostics using CRISPR-Cas13. Science. 360(6387), 444-448. Published online 2018/04/28 DOI: 10.1126/science.aas8836.
Smith, A. M., and Perelson, A. S. (2011). Influenza A virus infection kinetics: quantitative data and models. Wiley Interdiscip Rev Syst Biol Med. 3(4), 429-445. Published online 2011/01/05 DOI: 10.1002/wsbm.129.
Zetsche, B., Gootenberg, J. S., Abudayyeh, O. O., Slaymaker, I. M., Makarova, K. S., Essletzbichler, P., Volz, S. E., Joung, J., van der Oost, J., Regev, A., et al. (2015). Cpf1 is a single RNA-guided endonuclease of a class 2 CRISPR-Cas system. Cell. 163(3), 759-771. Published online 2015/10/01 DOI: 10.1016/j.cell.2015.09.038.

All publications, patent applications, patents and other references mentioned herein are expressly incorporated by reference in their entirety, to the same extent as if each were incorporated by reference individually. In case of conflict, the present specification, including definitions, will control.

The following statements provide a summary of some aspects of the inventive nucleic acids and methods described herein.

### Statements:

1. A method comprising:
(a) incubating a sample suspected of containing Influenza A or B RNA or virus with one or more Cas13 protein, at least one CRISPR guide RNA (crRNA), and at least one reporter RNA for a period of time sufficient to form at least one RNA cleavage product; and
(b) detecting reporter RNA cleavage product(s) with a detector.
   2. The method of statement 1, wherein the at least one CRISPR guide RNA (crRNA) binds a target site in at least one of an Influenza A or Influenza B nucleic acid.
   3. The method of statement 1 or 2, wherein one or more of the Cas13 proteins has a protein sequence with at least 95% sequence identity to any of SEQ ID NOs: 38-49.
   4. The method of any one of statements 1-3, wherein one or more of the Cas13 proteins has any one SEQ ID NOs: 38-49.
   5. The method of any one of statements 1 or 2, wherein the Influenza A RNA is from a variant of Influenza A.
   6. The method of any one of statements 1-5, wherein the at least one CRISPR guide RNA (crRNA) has a sequence segment with at least 95% sequence identity to any of SEQ ID NOs:1-37.
   7. The method of any one of statements 1-6, wherein the at least one CRISPR guide RNA (crRNA) has a sequence of any of SEQ ID NOs: 1-37.
   8. The method of any one of statements 1-7, wherein the at least one CRISPR guide RNA (crRNA) has a sequence segment with at least 95% sequence identity to any of SEQ ID NOs: 32, 34, 35, 36, or a combination thereof.
   9. The method of any one of statements 1-8, wherein the at least one CRISPR guide RNA (crRNA) has a sequence of any of SEQ ID NOs: 32, 34, 35, 36, or a combination thereof.
   10. The method of statement 9, wherein the at least one CRISPR guide RNA (crRNA) is a combination of SEQ ID NOs: 34 and 36.
   11. The method of any one of statements 1-10, wherein the at least one CRISPR guide RNA (crRNA) has a sequence segment with at least 95% sequence identity to any of SEQ ID NOs: 4, 8, 13, 16, 17, 21, 22, or a combination thereof.
   12. The method of statement 1, wherein the at least one CRISPR guide RNA (crRNA) has a sequence of any of SEQ ID NOs: 4, 8, 13, 16, 17, 21, 22, or a combination thereof.
   13. The method of statement 12, wherein the at least one CRISPR guide RNA (crRNA) is a combination of SEQ ID NOs: 8, 16, 21, and 22.
   14. The method of any one of statements 1, 2, 5-13, wherein one or more of the Cas13 protein is a Cas13a or Cas13b protein.
   15. The method of statement 1, wherein the at least one CRISPR guide RNA (crRNA) is two or more CRISPR guide RNAs (crRNAs).
   16. The method of statement 1, wherein the Cas13 protein is complexed with the at least one CRISPR guide RNA (crRNA) prior to incubation with the sample suspected of containing the target viral RNA.
   17. The method of statement 16, wherein the one or more of the Cas13 proteins is complexed with the at least one CRISPR guide RNA (crRNA) and prepared as a lyophilized bead.
   18. The method of statement 1, wherein the sample suspected of containing the target viral RNA is saliva, sputum, mucus, nasopharyngeal materials, blood, serum, plasma, urine, aspirate, biopsy tissue, or a combination thereof.
   19. The method of statement 1, wherein the sample suspected of containing RNA is a lysed biological sample.
   20. The method of statement 1, wherein cleavage of the reporter RNA produces a light signal, an electronic signal, an electrochemical signal, an electrostatic signal, a steric signal, a van der Waals interaction signal, a hydration signal, a Resonant frequency shift signal, or a combination thereof.
   21. The method of statement 1, wherein the reporter RNA reporter comprises at least one fluorophore and at least one fluorescence quencher.
   22. The method of statement 21, wherein the at least one fluorophore is Alexa 430, STAR 520, Brilliant Violet 510, Brilliant Violet 605, Brilliant Violet 610, or a combination thereof.
   23. The method of any of statements 1, 21, or 22, wherein the detector comprises a light detector, a fluorescence detector, a color filter, an electronic detector, an electrochemical signal detector, an electrostatic signal detector, a steric signal detector, a van der Waals interaction signal detector, a hydration signal detector, a Resonant frequency shift signal detector, or a combination.
   24. The method of statement 1, wherein the target viral RNA is detected when a signal from the reporter RNA cleavage product(s) is distinguishable from a control assay signal.
   25. The method of statement 24, wherein the control assay contains no target viral RNA.
   26. The method of statement 24, wherein the control assay contains viral RNA that is not the target viral RNA.
   27. The method of statement 1, wherein the sample comprises at least one RNA from a common cold coronavirus, SARS-CoV-2, hepatitis virus, respiratory syncytial virus (RSV), or human immunodeficiency virus (HIV).
   28. The method of statement 27, wherein the common cold coronavirus is at least one of strain NL63, OC43, or 229E.
   29. The method of statement 27, wherein the hepatitis virus is hepatitis C virus (HCV).
   30. The method of any one of statements 27-29, wherein at least one CRISPR guide RNAs can bind to at least one RNA from the common cold coronavirus, SARS-CoV-2, hepatitis virus, respiratory syncytial virus (RSV), or human immunodeficiency virus (HIV).
   31. A method comprising treating a subject with detectable Influenza A or B infection detected by the method of any of statements 1-26.
   32. A kit comprising a package containing at least one Cas13 protein, at least one CRISPR guide RNA (crRNA) that binds a target site in at least one of an Influenza A or Influenza B nucleic acid, at least one reporter RNA, and instructions for detecting and/or quantifying the target viral RNA in a sample.
   33. The kit of statement 32, wherein the at least one CRISPR guide RNA (crRNA) has a sequence with at least 95% sequence identity to any of SEQ ID NO: 1-37.
   34. The kit of any one of statements 32 or 33, wherein at least one of the CRISPR guide RNAs (crRNAs) has a sequence of any of SEQ ID NOs:1-37.
   35. The kit of any one of statements 32-34, wherein the at least one CRISPR guide RNA (crRNA) has a sequence segment with at least 95% sequence identity to any of SEQ ID NOs: 32, 34, 35, 36, or a combination thereof.
   36. The kit of any one of statements 32-35, wherein the at least one CRISPR guide RNA (crRNA) has any of SEQ ID NOs: 32, 34, 35, 36, or a combination thereof.
   37. The kit of statement 32, wherein the at least one CRISPR guide RNA (crRNA) is a combination of SEQ ID NOs: 34 and 36.
   38. The kit of statement 32, wherein the at least one CRISPR guide RNA (crRNA) has a sequence segment with at least 95% sequence identity to any of SEQ ID NOs: 4, 8, 13, 16, 17, 21, 22, or a combination thereof.
   39. The kit of statement 32, wherein the at least one CRISPR guide RNA (crRNA) has a sequence of any of SEQ ID NOs: 4, 8, 13, 16, 17, 21, 22, or a combination thereof.
   40. The kit of statement 28, wherein the at least one CRISPR guide RNA (crRNA) is a combination of SEQ ID NOs: 8, 16, 21, and 22.
   41. The kit of any one of statements 32-40, wherein the at least one CRISPR guide RNA (crRNA) is two or more CRISPR guide RNAs (crRNAs).
   42. The kit of any one of statements 32-41, wherein the Cas13 protein is complexed with the at least one CRISPR guide RNA (crRNA).
   43. The kit of any one of statements 32-42, wherein the one or more of the Cas13 proteins is complexed with the at least one CRISPR guide RNA (crRNA) and prepared as a lyophilized bead.
   44. The kit of any one of statements 32, 42, or 43, wherein the Cas13 protein is a Cas13a or Cas13b protein.
   45. The kit of statement 32, wherein the reporter RNA reporter comprises at least one fluorophore and at least one fluorescence quencher.
   46. The kit of statement 45, wherein the at least one fluorophore is Alexa 430, STAR 520, Brilliant Violet 510, Brilliant Violet 605, Brilliant Violet 610, or a combination thereof.
   47. The kit of any one of statements 32 or 43, further comprising a sample chamber, assay mixture reaction chamber, or a combination thereof.
   48. The kit of statement 43, wherein the lyophilized bead is included in the assay mixture reaction chamber.
   49. The kit of statement 32, further comprising a detector.

1. A method comprising:
(a) incubating a sample suspected of containing Influenza A or B RNA or virus with one or more Cas13 protein, at least one CRISPR guide RNA (crRNA), and at least one reporter RNA for a period of time sufficient to form at least one RNA cleavage product; and
(b) detecting reporter RNA cleavage product(s) with a detector.

2. The method of claim 1, wherein the sample comprises RNA from a variant of Influenza A.

3. The method of claim 1, wherein the at least one CRISPR guide RNA (crRNA) has a sequence segment with at least 95% sequence identity to any of SEQ ID NOs:1-37.

4. The method of claim 1, wherein the at least one CRISPR guide RNA (crRNA) has a sequence of any of SEQ ID NOs: 1-37.

5. The method of claim 1, wherein the at least one CRISPR guide RNA (crRNA) has a sequence segment with at least 95% sequence identity to any of SEQ ID NOs: 32, 34, 35, 36, or a combination thereof.

6. The method of claim 1, wherein the at least one CRISPR guide RNA (crRNA) has a sequence of any of SEQ ID NOs: 32, 34, 35, 36, or a combination thereof.

7. The method of claim 6, wherein the at least one CRISPR guide RNA (crRNA) is a combination of SEQ ID NOs: 34 and 36.

8. The method of claim 1, wherein the at least one CRISPR guide RNA (crRNA) has a sequence segment with at least 95% sequence identity to any of SEQ ID NOs: 4, 8, 13, 16, 17, 21, 22, or a combination thereof.

9. The method of claim 1, wherein the at least one CRISPR guide RNA (crRNA) has a sequence of any of SEQ ID NOs: 4, 8, 13, 16, 17, 21, 22, or a combination thereof.

10. The method of claim 9, wherein the at least one CRISPR guide RNA (crRNA) is a combination of SEQ ID NOs: 8, 16, 21, and 22.

11. The method of claim 1, wherein one or more of the Cas13 protein is a Cas13a or Cas13b protein.

12. The method of claim 1, wherein the at least one CRISPR guide RNA (crRNA) is two or more CRISPR guide RNAs (crRNAs).

13. The method of claim 1, wherein the Cas13 protein is complexed with the at least one CRISPR guide RNA (crRNA) prior to incubation with the sample suspected of containing the target viral RNA.

14. The method of claim 13, wherein the one or more of the Cas13 proteins is complexed with the at least one CRISPR guide RNA (crRNA) and prepared as a lyophilized bead.

15. The method of claim 1, wherein the sample suspected of containing the target viral RNA is saliva, sputum, mucus, nasopharyngeal materials, blood, serum, plasma, urine, aspirate, biopsy tissue, or a combination thereof.

16. The method of claim 1, wherein the sample suspected of containing RNA is a lysed biological sample.

17. The method of claim 1, wherein cleavage of the reporter RNA produces a light signal, an electronic signal, an electrochemical signal, an electrostatic signal, a steric signal, a van der Waals interaction signal, a hydration signal, a Resonant frequency shift signal, or a combination thereof.

18. The method of claim 1, wherein the reporter RNA reporter comprises at least one fluorophore and at least one fluorescence quencher.

19. The method of claim 18, wherein the at least one fluorophore is Alexa 430, STAR 520, Brilliant Violet 510, Brilliant Violet 605, Brilliant Violet 610, or a combination thereof.

20. The method of claim 1, wherein the detector comprises a light detector, a fluorescence detector, a color filter, an electronic detector, an electrochemical signal detector, an electrostatic signal detector, a steric signal detector, a van der Waals interaction signal detector, a hydration signal detector, a Resonant frequency shift signal detector, or a combination.

21. The method of claim 1, wherein the target viral RNA is detected when a signal from the reporter RNA cleavage product(s) is distinguishable from a control assay signal.

22. The method of claim 21, wherein the control assay contains no target viral RNA.

23. The method of claim 21, wherein the control assay contains viral RNA that is not the target viral RNA.

24. The method of claim 1, wherein the sample comprises at least one RNA from a common cold coronavirus, SARS-CoV-2, hepatitis virus, respiratory syncytial virus (RSV), or human immunodeficiency virus (HIV).

25. The method of claim 24, wherein the common cold coronavirus is at least one of strain NL63, OC43, or 229E.

26. The method of claim 24, wherein the hepatitis virus is hepatitis C virus (HCV).

27. The method of claim 24, wherein the at least one CRISPR guide RNAs can bind to the at least one RNA from the common cold coronavirus, SARS-CoV-2, hepatitis virus, respiratory syncytial virus (RSV), or human immunodeficiency virus (HIV).

28. A method comprising treating a subject with detectable Influenza A or B infection detected by the method of claim 1.

29-47. (canceled)


SEQUENCE LISTING (66 sequences)

SEQ ID NO: 1 - RNA, 50 nt, synthetic construct
  source 1..50: mol_type = other RNA
gaccaccccaaaaatgaaggggactaaaacgagccgaacggctgcattga

SEQ ID NO: 2 - RNA, 50 nt, synthetic construct
  source 1..50: mol_type = other RNA
gaccaccccaaaaatgaaggggactaaaacctggacagtggtgaacagta

SEQ ID NO: 3 - RNA, 50 nt, synthetic construct
  source 1..50: mol_type = other RNA
gaccaccccaaaaatgaaggggactaaaaccctgaagtctgcttaaagtg

SEQ ID NO: 4 - RNA, 50 nt, synthetic construct
  source 1..50: mol_type = other RNA
gaccaccccaaaaatgaaggggactaaaacaatttttccctagtagttca

SEQ ID NO: 5 - RNA, 50 nt, synthetic construct
  source 1..50: mol_type = other RNA
gaccaccccaaaaatgaaggggactaaaacccctagtagttcatatagga

SEQ ID NO: 6 - RNA, 50 nt, synthetic construct
  source 1..50: mol_type = other RNA
gaccaccccaaaaatgaaggggactaaaacgtgccatagaggagacacac

SEQ ID NO: 7 - RNA, 50 nt, synthetic construct
  source 1..50: mol_type = other RNA
gaccaccccaaaaatgaaggggactaaaactatggccataattaagaagt

SEQ ID NO: 8 - RNA, 50 nt, synthetic construct; identical to SEQ ID NO: 4
  source 1..50: mol_type = other RNA

SEQ ID NO: 9 - RNA, 50 nt, synthetic construct
  source 1..50: mol_type = other RNA
gaccaccccaaaaatgaaggggactaaaactcagctgacatgagtattgg

SEQ ID NO: 10 - RNA, 50 nt, synthetic construct
  source 1..50: mol_type = other RNA
gaccaccccaaaaatgaaggggactaaaaccagctgacatgagtattgga

SEQ ID NO: 11 - RNA, 50 nt, synthetic construct
  source 1..50: mol_type = other RNA
gaccaccccaaaaatgaaggggactaaaacgacatgagtattggagtaac

SEQ ID NO: 12 - RNA, 50 nt, synthetic construct; identical to SEQ ID NO: 3
  source 1..50: mol_type = other RNA

SEQ ID NO: 13 - RNA, 50 nt, synthetic construct
  source 1..50: mol_type = other RNA
gaccaccccaaaaatgaaggggactaaaacgagtcaggaaggctaataga

SEQ ID NO: 14 - RNA, 49 nt, synthetic construct
  source 1..49: mol_type = other RNA
gaccaccccaaaaataaggggactaaaaccggttggaatttctagcatg

SEQ ID NO: 15 - RNA, 50 nt, synthetic construct
  source 1..50: mol_type = other RNA
gaccaccccaaaaatgaaggggactaaaacggttggaatttctagcatgg

SEQ ID NO: 16 - RNA, 50 nt, synthetic construct
  source 1..50: mol_type = other RNA
gaccaccccaaaaatgaaggggactaaaacatatgaagcaatcgaggagt

SEQ ID NO: 17 - RNA, 50 nt, synthetic construct
  source 1..50: mol_type = other RNA
gaccaccccaaaaatgaaggggactaaaactatgaagcaatcgaggagtg

SEQ ID NO: 18 - RNA, 50 nt, synthetic construct
  source 1..50: mol_type = other RNA
gaccaccccaaaaatgaaggggactaaaaccctgcctgcttgtgtgtatg

SEQ ID NO: 19 - RNA, 50 nt, synthetic construct
  source 1..50: mol_type = other RNA
gaccaccccaaaaatgaaggggactaaaaccggcaatggtgtttggatag

SEQ ID NO: 20 - RNA, 50 nt, synthetic construct
  source 1..50: mol_type = other RNA
gaccaccccaaaaatgaaggggactaaaacaacgtacgttctttctatca

SEQ ID NO: 21 - RNA, 50 nt, synthetic construct
  source 1..50: mol_type = other RNA
gaccaccccaaaaatgaaggggactaaaactatacagagattcgcttgga

SEQ ID NO: 22 - RNA, 50 nt, synthetic construct
  source 1..50: mol_type = other RNA
gaccaccccaaaaatgaaggggactaaaacatacagagattcgcttggag

SEQ ID NO: 23 - RNA, 50 nt, synthetic construct
  source 1..50: mol_type = other RNA
gaccaccccaaaaatgaaggggactaaaactactacaaaaatcccggaac

SEQ ID NO: 24 - RNA, 50 nt, synthetic construct
  source 1..50: mol_type = other RNA
gaccaccccaaaaatgaaggggactaaaacagatagcatattaaacattc

SEQ ID NO: 25 - RNA, 50 nt, synthetic construct
  source 1..50: mol_type = other RNA
gaccaccccaaaaatgaaggggactaaaacatttatcccatgtagctcac

SEQ ID NO: 26 - RNA, 50 nt, synthetic construct
  source 1..50: mol_type = other RNA
gaccaccccaaaaatgaaggggactaaaacgacatcattctgccgcatat

SEQ ID NO: 27 - RNA, 50 nt, synthetic construct
  source 1..50: mol_type = other RNA
gaccaccccaaaaatgaaggggactaaaacagacatcttctagcttccat

SEQ ID NO: 28 - RNA, 50 nt, synthetic construct
  source 1..50: mol_type = other RNA
gaccaccccaaaaatgaaggggactaaaactcccagtgcagattcgatct

SEQ ID NO: 29 - RNA, 50 nt, synthetic construct
  source 1..50: mol_type = other RNA
gaccaccccaaaaatgaaggggactaaaacatcccagtgcagattcgatc

SEQ ID NO: 30 - RNA, 50 nt, synthetic construct
  source 1..50: mol_type = other RNA
gaccaccccaaaaatgaaggggactaaaactatcccagtgcagattcgat

SEQ ID NO: 31 - RNA, 50 nt, synthetic construct
  source 1..50: mol_type = other RNA
gaccaccccaaaaatgaaggggactaaaacttatcccagtgcagattcga

SEQ ID NO: 32 - RNA, 50 nt, synthetic construct
  source 1..50: mol_type = other RNA
gaccaccccaaaaatgaaggggactaaaacgtcgtgcattataggaaagc

SEQ ID NO: 33 - RNA, 50 nt, synthetic construct
  source 1..50: mol_type = other RNA
gaccaccccaaaaatgaaggggactaaaacttcatacccaaccatagagt

SEQ ID NO: 34 - RNA, 50 nt, synthetic construct
  source 1..50: mol_type = other RNA
gaccaccccaaaaatgaaggggactaaaacgacagcattcttcttacagc

SEQ ID NO: 35 - RNA, 50 nt, synthetic construct
  source 1..50: mol_type = other RNA
gaccaccccaaaaatgaaggggactaaaacgataagactcccaccgcagt

SEQ ID NO: 36 - RNA, 50 nt, synthetic construct
  source 1..50: mol_type = other RNA
gaccaccccaaaaatgaaggggactaaaacgctgtacacttcaaccacat

SEQ ID NO: 37 - RNA, 50 nt, synthetic construct
  source 1..50: mol_type = other RNA
gaccaccccaaaaatgaaggggactaaaactgcctgctgtacacttcaac

SEQ ID NO: 38 - protein, 1177 aa, Leptotrichia wadei
MKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKLENEFLENVKAMLEYSE

SEQ ID NO: 39 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 40 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 41 - protein, 1120 aa, Leptotrichia seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 42 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 43 - protein, 1437 aa, Lachnospiraceae bacterium
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 44 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 45 - protein, 1159 aa, Leptotrichia buccalis; identical to SEQ ID NO: 40

SEQ ID NO: 46 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 47 - protein, 1127 aa, Prevotella buccae
MQKQDKLFVDRKKNAIFAFPKYITIMENQEKPEPIYYELTDKHFWAAFLNLARHNVYTTINHINRRLEIAELKDDGYMMDIKGSWNEQAKKLDKKVRLRDLIMKHFPFLEAAAYEITNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYDAEEEPFKNTLVRHQDRFPYFVLRYFDLNEIFEQLRFQIDLGTYHFSIYNKRIGDEDEVRHLTHHLYGFARIQDFAQQNQPEVWRKLVKDLDYFEASQEPYIPKTAPHYHLENEKIGIKFCSTHNNLFPSLKTEKTCNGRSKFNLGTQFTAEAFLSVHELLPMMFYYLLLTKDYSRKESADKVEGIIRKEISNIYAIYDAFANGEINSIADLTCRLQKTNILQGHLPKQMISILEGRQKDMEKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNKLKPQKGQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKVLAKDRRLNGLLSFAETTDIDLEKNPITKLSVDHELIKYQTTRISIFEMTLGLEKKLINKYPTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 48 - protein, 1232 aa, Bergeyella zoohelcum
XENKTSLGNNIYYNPFKPQDKSYFAGYFNAAXENTDSVFRELGKRLKGKEYTSENFFDAIFKENISLVEYERYVKLLSDYFPXARLLDKKEVPIKERKENFKKNFKGIIKAVRDLRNFYTHKEHGEVEITDEIFGVLDEXLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFXATHEIFSHLAYKKLKRKVRTAEINYGEAENAEQLSVYAKETLXXQXLDELSKVPDVVYQNLSEDVQKTFIEDWNEYLKENNGDVGTXEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSXNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFXPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFXDEKPTIIKGKTFKGNEALFADWFRYYKEYQNFQTFYDTENYPLVELEKKQADRKRKTKIYQQKKNDVFTLLXAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIANKFAHNQLPKKEFWDYCQEKYGKIEKEKTYAEYFAEVFKKEKEALIKLEHHHHHH

SEQ ID NO: 49 - protein, 1127 aa, Prevotella buccae
MQKQDKLFVDRKKNAIFAFPKYITIMENQEKPEPIYYELTDKHFWAAFLNLARHNVYTTINHINRRLEIAELKDDGYMMGIKGSWNEQAKKLDKKVRLRDLIMKHFPFLEAAAYEITNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYDAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSTHNNLFPSLKREKTCNGRSKFNLGTQFTAEAFLSVHELLPMMFYYLLLTKDYSRKESADKVEGIIRKEISNIYAIYDAFANNEINSIADLTCRLQKTNILQGHLPKQMISILEGRQKDMEKEAERKIGEMIDDTQRRLDLLCKQTNQKIRIGKRNAGLLKSGKIADWLVSDMMRFQPVQKDTNNAPINNSKANSTEYRMLQHALALFGSESSRLKAYFRQMNLVGNANPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNKLKPQKGQFLDKKERVELWQKNKELFKNYPSEKNKTDLAYLDFLSWKKFERELRLIKNQDIVTWLMFKELFKTTTVEGLKIGEIHLRDIDTNTANEESNNILNRIMPMKLPVKTYETDNKGNILKERPLATFYIEETETKVLKQGNFKVLAKDRRLNGLLSFAETTDIDLEKNPITKLSVDYELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 50 - skipped

SEQ ID NO: 51 - DNA, 2341 bp, Influenza A
  source 1..2341: mol_type = other DNA
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaatgacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaagaatgatgatgttgatcaaagcttgattattgctgctaggaacatagtgagaagagctgcagtatcagcagacccactagcatctttattggagatgtgccacagcacacagattggtggaattaggatggtagacatccttaagcagaacccaacagaagagcaagccgtgggtatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagttagaggtgatctgaatttcgtcaatagggcgaatcagcgactgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccggttcttgagagtccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtgagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactacaacaaggccacgaagagactcacagttctcggaaaggatgctggcactttaaccgaagacccagatgaaggcacagctggagtggagtccgctgttctgaggggattcctcattctgggcaaagaagacaggagatatgggccagcattaagcatcaatgaactgagcaaccttgcgaaaggagagaaggctaatgtgctaattgggcaaggagacgtggtgttggtaatgaaacgaaaacgggactctagcatacttactgacagccagacagcgaccaaaagaattcggatggccatcaattagtgtcgaatagtttaaaaacgaccttgtttctact

SEQ ID NO: 52 - DNA, 2341 bp, Influenza A
  source 1..2341: mol_type = other DNA
agcgaaagcaggcaaaccatttgaatggatgtcaatccgaccttacttttcttaaaagtgccagcacaaaatgctataagcacaactttcccttataccggagaccctccttacagccatgggacaggaacaggatacaccatggatactgtcaacaggacacatcagtactcagaaaaggcaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactttaaatagaaaccagcctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaggaaacagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttgaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcattgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacatggaatcaatatgagcaagaaaaagtcttacataaacagaacaggtacatttgaattcacaagttttttctatcgttatgggtttgttgccaatttcagcatggagcttcccagttttggtgtgtctgggagcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgttcctact

SEQ ID NO: 53 - DNA, 2233 bp, Influenza A
  source 1..2233: mol_type = other DNA
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagatttccacttcatcaatgagcaaggcgagtcaataatcgtagaacttggtgatcctaatgcacttttgaagcacagatttgaaataatcgagggaagagatcgcacaatggcctggacagtagtaaacagtatttgcaacactacaggggctgagaaaccaaagtttctaccagatttgtatgattacaaggaaaatagattcatcgaaattggagtaacaaggagagaagttcacatatactatctggaaaaggccaataaaattaaatctgagaaaacacacatccacattttctcgttcactggggaagaaatggccacaaaggccgactacactctcgatgaagaaagcagggctaggatcaaaaccaggctattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaaaaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagttcaacaaggcatgcgaactgacagattcaagctggatagagcttgatgagattggagaagatgtggctccaattgaacacattgcaagcatgagaaggaattatttcacatcagaggtgtctcactgcagagccacagaatacataatgaagggggtgtacatcaatactgccttacttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacacaaatgggagaagtactgtgttcttgagataggagatatgcttctaagaagtgccataggccaggtttcaaggcccatgttcttgtatgtgaggacaaatggaacctcaaaaattaaaatgaaatggggaatggagatgaggcgttgtctcctccagtcacttcaacaaattgagagtatgattgaagctgagtcctctgtcaaagagaaagacatgaccaaagagttctttgagaacaaatcagaaacatggcccattggagagtctcccaaaggagtggaggaaagttccattgggaaggtctgcaggactttattagcaaagtcggtatttaacagcttgtatgcatctccacaactagaaggattttcagctgaatcaagaaaactgcttcttatcgttcaggctcttagggacaatctggaacctgggacctttgatcttggggggctatatgaagcaattgaggagtgcctaattaatgatccctgggttttgcttaatgcttcttggttcaactccttccttacacatgcattgagttagttgtggcagtgctactatttgctatccatactgtccaaaaaagtaccttgtttctact

SEQ ID NO: 54 - DNA, 1722 bp, Influenza A
  source 1..1722: mol_type = other DNA
agcaaaagcaggggaaaataaaaacaaccaaaatgaaggcaaacctactggtcctgttatgtgcacttgcagctgcagatgcagacacaatatgtataggctaccatgcgaacaattcaaccgacactgttgacacagtgctcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaccaaaggagtaacggcagcatgctcccatgcggggaaaagcagtttttacagaaatttgctatggctgacggagaaggagggctcatacccaaagctgaaaaattcttatgtgaacaagaaagggaaagaagtccttgtactgtggggtattcatcacccgtctaacagtaaggatcaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaaggtatgctttcgcactgagtagaggctttgggtccggcatcatcacctcaaacgcatcaatgcatgagtgtaacacgaagtgtcaaacacccctgggagctataaacagcagtctccctttccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtacggttatcatcatcagaatgaacagggatcaggctatgcagcggatcaaaaaagcacacaaaatgccattaacgggattacaaacaaggtgaactctgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaa

SEQ ID NO: 55 - DNA, 1565 bp, Influenza A
  source 1..1565: mol_type = other DNA
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcacagaacttaaactcagtgattatgagggacggttgatccaaaacagcttaacaatagagagaatggtgctctctgcttttgacgaaaggagaaataaatacctggaagaacatcccagtgcggggaaggatcctaagaaaactggaggacctatatacagaagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagggctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccgggacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattgagcttcatcaaagggacgaaggtggtcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccgttatggcagcattcactgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 56 - DNA, 1413 bp, Influenza A
  source 1..1413: mol_type = other DNA
agcgaaagcaggggtttaaaatgaatccaaatcagaaaataataaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaggcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaattggcatggttcgaaccggccatgggtgtctttcgatcaaaacctggattatcaaataggatacatctgcagtggggttttcggtgacaacccgcgtcccaaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgtgaggcaagatgttgtggcaatgactgattggtcagggtatagcgggagtttcgttcaacatcctgagctaacagggctagactgtataaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcaccattgacaagtagtctgttcaaaaaactccttgtttctact

SEQ ID NO: 57 - DNA, 1027 bp, Influenza A
  source 1..1027: mol_type = other DNA
agcgaaagcaggtagatattgaaagatgagtcttctaaccgaggtcgaaacgtacgttctctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaacccactaatcagacatgagaacagaatggttttagccagcactacagctaaggctatggagcaaatggctggatcgagtgagcaagcagcagaggccatggaggttgctagtcaggctaggcaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgatattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 58 - DNA, 890 bp, Influenza A
  source 1..890: mol_type = other DNA
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggcctctgtacctgcgtcgcgttacctaaccgacatgactcttgaggaaatgtcaagggaatggtccatgctcatacccaagcagaaagtggcaggccctctttgtatcagaatggaccaggcgatcatggataaaaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaaggtaacagagaatagttttgagcaaataacatttatgcaagccttacatctattgcttgaagtggagcaagagataagaactttctcatttcagcttatttaataataaaaaacacccttgtttctact

SEQ ID NO: 59 - DNA, 2328 bp, Influenza B
  source 1..2328: mol_type = other DNA
gatgaatataaatccttattttctcttcatagatgtgcccatacaggcagcaatttcaacaacattcccatacactggtgttcccccttattcccatggaacgggaacaggctacacaatagacacagtgatcagaacacatgagtactcaaacaaggggaaacagtacatttctgatgttacaggatgcacaatggtagatccaacaaatggaccattacccgaagataatgagccgagtgcctatgcgcaattagattgcgttttggaggctttggatagaatggatgaagaacacccaggtctgtttcaagcagcctcacagaatgctatggaggccctaatggtcacaactgtagacaaattaacccaggggagacagacttttgattggacagtatgcagaaaccaacctgctgcaacggcactgaacacaacaataacctcttttaggttgaatgatttaaatggagccgacaaaggtggattagtacctttttgccaggatatcattgattcattagacagacctgaaatgactttcttctcagtaaagaatataaagaaaaaattgcctgccaaaaacagaaagggtttcctcataaagaggataccaatgaaggtaaaagacaaaataaccaaagtggaatacatcaaaagagcattatcattaaacacaatgacaaaagacgctgaaagaggcaaactaaaaagaagagcgattgccaccgctggaatacaaatcagaggatttgtattagtagttgaaaacttggctaaaaatatatgtgaaaatctagaacaaagtggtttaccagtaggtggaaacgagaagaaagccaaactgtcaaacgcagtggccaaaatgctcagtaactgcccaccaggagggatcagcatgacagtaacaggagacaatactaaatggaatgaatgtttaaacccaagaatctttttggctatgactgaaagaataaccagagacagcccaatttggttcagggatttttgtagtatagcaccggtcctgttctccaataagatagccagattggggaaagggtttatgataacaagcaaaacaaaaagactgaaggctcaaataccttgtcctgatctgtttagtataccattagaaagatataatgaagaaacaagggcaaaattgaaaaagctaaaaccattcttcaatgaagaaggaactgcatctttgtcgcctgggatgatgatgggaatgtttaacatgctatctaccgtgttgggagtagccgcactaggtatcaagaacattggaaacaaagaatacttatgggatggactgcaatcttctgatgattttgctctatttgttaatgcaaaggatgaagaaacatgtatggaaggaataaacgacttttaccgaacatgtaaattattgggaataaacatgagcaaaaagaaaagttactgtaatgagactggaatgtttgaatttacaagcatgttctacagagatggatttgtatctaattttgcaatggaactcccttcatttggggttgctggagtaaatgaatcagcagatatggcaatagggatgacaataataaagaacaacatgatcaacaatggaatgggtccggcaacagcacaaacagccatacagttattcatagctgattatagatacacctacaaatgccacaggggagattccaaagtagaaggaaagagaatgaaaatcataaaggagttatgggaaaacactaaaggaagagatggtctattagtagcagatggtgggcccaacatttacaatttgagaaacttgcatattccagaaatagtattaaagtataacctaatggaccctgaatacaaagggcgattacttcatcctcaaaatccctttgtgggacatttgtctattgagggcatcaaagaggcagatataaccccagcacatggtccagtaaagaaaatggactacgatgcggtgtctggaactcatagttggagaaccaaaagaaacagatctatactaaacactgatcagaggaacatgattcttgaggaacaatgctacgctaagtgttgcaacctatttgaggcctgttttaacagtgcatcatacaggaagccagtgggtcaacatagcatgcttgaggctatggcccacagattaagaatggatgcacgattagattatgaatcaggaagaatgtcaaaggatgattttgagaaagcaatggctcaccttggtgagattgggtacatataagcttcgaagatgtctatggggttattggtcatcattgaatacatgcgatacacaaatgattaaaatga

SEQ ID NO: 60 - DNA, 2352 bp, Influenza B
  source 1..2352: mol_type = other DNA
gatgacattggccaaaattgaattgttaaaacaactgctaagggacaatgaagccaaaacagttttgaagcaaacaacggtagaccaatataacataataagaaaattcaatacatcaaggattgaaaagaatccttcactaaggatgaagtgggccatgtgttctaattttcccttggctctaaccaagggcgatatggcaaatagaatccccttggaatacaaaggaatacaacttaaaacaaatgctgaagacataggaaccaaaggccaaatgtgctcaatagcagcagttacttggtggaatacatatggaccaataggagatactgaaggtttcgaaagggtctacgaaagcttttttctcagaaaaatgagacttgacaacgccacttggggccgaataacttttggcccagttgaaagagtgagaaaaagggtactgctaaaccctctcaccaaggaaatgcctccagatgaggcgagcaatgtgataatggaaatattgttccctaaagaagcaggaataccaagagaatccacttggatacatagggaactgataaaagaaaaaagagaaaaattgaaaggaacaatgataactccaatcgtactggcatacatgcttgaaagagaactggttgctcgaagaagattcttgccagtggcaggagcaacatcagctgagttcatagaaatgctacactgcttacaaggtgaaaattggagacaaatatatcacccaggagggaataaattaactgagtctaggtctcaatcaatgatagtagcttgtagaaaaataatcagaagatcaatagtcgcttcaaacccactggagctagctgtagaaattgcaaacaagactgtgatagatactgaacctttaaagtcatgtctggcagccatagacggaggtgatgtagcttgtgacataataagagctgcattaggactaaagatcagacaaagacaaagatttggacggcttgagctaaaaagaatatcaggaagaggattcaaaaatgatgaagaaatattaatagggaacggaacaatacagaagattggaatatgggacggggaagaggagttccatgtaagatgtggtgaatgcaggggaatattaaaaaagagtaaaatgaaactggaaaaactactgataaattcagccaaaaaggaggatatgagagatttaataatcttatgcatggtattttctcaagacactaggatgttccaaggggtgagaggagaaataaattttcttaatcgagcaggccaacttttatctccaatgtaccaactccaacgatattttttgaataggagcaacgacctttttgatcaatgggggtatgaggaatcacccaaagcaagtgaactacatgggataaatgaatcaatgaatgcatctgactatacattgaaaggggttgtagtgacaagaaatgtaattgacgactttagctctactgaaacagaaaaagtatccataacaaaaaatcttagtttaataaaaaggactggggaagtcataatgggagctaatgacgtgagtgaattagaatcacaagcacagctgatgataacatatgatacacctaagatgtgggaaatgggaacaaccaaagaactggtgcaaaacacttatcaatgggtgctaaaaaacttggtaacactgaaggctcagtttcttctaggaaaagaggacatgttccaatgggatgcatttgaagcatttgagagcataattcctcagaaaatggctggtcagtacagtggatttgcaagagcagtgctcaaacaaatgagagaccaggaggttatgaaaactgaccagttcataaagttgttgcctttttgtttctcaccaccaaaattaaggagcaatggggagccttatcaattcttaaaacttgtattgaaaggaggaggggaaaatttcatcgaagtaaggaaagggtcccctctattttcctataatccacaaacagaggtcctaactatatgcggcagaatgatgtcattaaaagggaaaattgaagatgaagaaaggaatagatcaatggggaatgcagtattagcaggctttctcgttagtggcaagtatgacccagatcttggagatttcaaaactattgaagaacttgaaaagctgaaaccgggggaaaaggcaaacatcttactttatcaaggaaagccagttaaagtagttaaaaggaaaaggtatagtgctttgtccaatgacatttcacaaggaattaagagacaaagaatgacagttgagtccatggggtgggccttgagctaatataaatttatccattaattcaatgaacgcaattgagt

SEQ ID NO: 61 - DNA, 2213 bp, Influenza B
  source 1..2213: mol_type = other DNA
tttgatttgtcataatggatacttttattacaagaaacttccagactacaataatacaaaaggccaaaaacacaatggcagaatttagtgaagatcctgaattacaaccagcaatgctattcaatatctgcgtccatctagaggtttgctatgtaataagtgacatgaattttcttgacgaagaaggaaaagcatatacagcattagaaggacaagggaaagaacaaaatttgagaccacaatatgaagtaattgagggaatgccaagaaccatagcatggatggtccaaagatccttagctcaagagcatggaatagagactcccaagtatctggctgatttgtttgattataaaaccaagagatttatagaagttggaataacaaaaggattggctgatgattacttttggaaaaagaaagaaaagttgggaaatagcatggaactgatgatattcagctacaatcaagactactcgttaagtaatgaatcctcattggatgaggaagggaaagggagagtgctaagcagactcacagaacttcaggctgaattaagtctgaaaaacctatggcaagttctcataggagaagaagatgttgaaaagggaattgactttaaacttggacaaacaatatctagactaagggatatatctgttccagctggtttctccaattttgaaggaatgaggagctacatagacaatatagacccaaaaggagcaatagagagaaatctagcaaggatgtctcccttagtatcagtcacacctaaaaagttaacatgggaggacctaagaccaatagggcctcacatttacaaccatgagctaccagaagttccatataatgcctttcttctaatgtctgatgaactggggctggccaatatgactgagggaaagtccaaaaaaccgaagacattagccaaagaatgtctagaaaagtactcaacactacgggatcaaactgacccaatattaataatgaaaagcgaaaaagctaacgaaaatttcctatggaagctttggagagactgtgtaaatacaataagtaatgaggaaatgaataacgagttacagaaaaccaattatgccaagtgggccacaggggatggattaacataccagaaaataatgaaagaagtagcaatagatgacgaaacaatgtgccaagaagagcctaaaatccctaacaaatgtagagtggctgcttgggttcaaacagagatgaatctattgagcactctgacaagtaaaagagctctggacctaccagaaatagggccagacgtagcacccgtggagcatgtagggagtgaaagaaggaaatactttgttaatgaaatcaactactgtaaggcctctacagttatgatgaagtatgtgctttttcacacttcattgttgaatgaaagcaatgccagcatgggaaaatacaaagtaataccaataaccaatagagtagtaaatgaaaaaggagaaagtttcgacatgctttatggtctggcggttaaaggacaatctcatctgaggggagatactgatgttgtaacagttgtaactttcgaatttagtagtacagacccaagagtggactcaggaaagtggccaaaatatactgtgtttaggattggctccctatttgtgagtgggagggaaaaatctgtgtacctgtattgccgagtgaatggcacaaataagatccaaatgaaatggggaatggaagctagaagatgtctgcttcaatcaatgcaacaaatggaagcaattgttgaacaggaatcatcgatacaaggatatgacatgaccaaagcttgtttcaagggagacagagtaaatagccccaaaactttcagtattggaactcaagaaggaaaactagtaaaaggatcctttggaaaagcactaagagtaatatttactaaatgtttgatgcactatgtatttggaaatgcccaattggaggggtttagtgccgagtctaggagacttctactgttgattcaagcattaaaggacagaaagggcccttgggtgttcgacttagagggaatgtattctggaatagaagaatgtattagtaacaacccttgggtaatacagagtgcatactggttcaatgaatggttgggctttgaaaaggaggggagtaaagtgttagaatcagtggatgaaataatggatgaataaaaggacatggtactcaat

SEQ ID NO: 62 - DNA, 1843 bp, Influenza B
  source 1..1843: mol_type = other DNA
atatccacaaaatgaaggcaataattgtactactcatggtagtaacatccaatgcagatcgaatctgcactgggataacatcgtcaaactcaccccatgtggtcaaaactgctactcaaggggaggtcaatgtgactggtgtgataccactgacaacaacacccaccaaatctcattttgcaaatctcaaaggaacaaaaaccagagggaaactatgcccaaaatgcctcaactgcacagatctggacgtggccttgggcagaccaaaatgcacggggaacataccctcggcaaaagtttcaatactccatgaagtcagacctgttacatctgggtgctttcctataatgcacgacagaacaaaaattagacagctgcccaatcttctcagaggatacgaacatatcaggttatcaactcataacgttatcaatgcagaaaaggcaccaggaggaccctacaaaattggaacctcagggtcttgccctaacgttaccaatggaaacggatttttcgcaacaatggcttgggccgtcccaaaaaacgacaacaacaaaacagcaacaaattcattaacaatagaagtaccatacatttgtacagaaggagaagaccaaattaccgtttgggggttccactctgataacgaagcccaaatggcaaaactctatggggactcaaagccccagaagttcacctcatctgccaacggagtgaccacacattacgtttcacagattggtggcttcccaaatcaaacagaagacggaggactaccacaaagtggtagaattgttgttgattacatggtgcaaaaatctgggaaaacaggaacaattacctatcaaagaggtattttattgcctcaaaaagtgtggtgcgcaagtggcaggagcaaggtaataaaaggatccttgcctttaattggagaagcagattgcctccacgaaaaatacggtggattaaacaaaagcaagccttactacacaggggaacatgcaaaggccataggaaattgcccaatatgggtgaaaacacccttgaagctggccaatggaaccaaatatagacctcctgcaaaactattaaaggaaagaggtttcttcggagctattgctggtttcttagaaggaggatgggaaggaatgattgcaggttggcacggatacacatcccatggggcacatggagtagcagtggcagcagaccttaagagtactcaagaagccataaacaagataacaaaaaatctcaactctttgagtgagctggaagtaaagaatcttcaaagactaagcggtgccatggatgaactccacaacgaaatactagaactagacgagaaagtggatgatctcagagctgatacaataagctcacaaatagaactcgcagtcttgctttccaatgaaggaataataaacagtgaagatgagcatctcttggcgcttgaaagaaagctgaagaaaatgctgggcccctctgctgtagagatagggaatggatgcttcgaaaccaaacacaagtgcaaccagacctgtctcgacagaatagctgctggtacctttgatgcaggagaattttctctccccacttttgattcactgaatattactgctgcatctttaaatgacgatggattggataatcatactatactgctttactactcaactgctgcctccagtttggctgtaacattgatgatagctatctttgttgtttatatggtctccagagacaatgtttcttgctccatctgtctataaggaaagttaagccctgtattttcctttattgtagtgcttgtttgcttgttaccattacaaaaaaacgttattga

SEQ ID NO: 63 - DNA, 1809 bp, Influenza B
  source 1..1809: mol_type = other DNA
tttcttgtgaacttcaagtgctaacaaaagaactgaaaatcaaaatgtccaacatggatattgacggtatcaacactgggacaattgacaaagcaccggaagaaataacttctggaaccagtgggacaaccagaccaatcatcagaccagcaacccttgccccaccaagcaacaaacgaacccggaacccatccccggaaagagcaaccacaatcagtgaagctgatgtcggaaggaaaaaccaaaagaaacagaccccgacagagataaagaagagcgtctacaatatggtagtgaaactgggtgaattctataaccagatgatggtcaaagctggacttaacgatgacatggagagaaacctaattcaaaatgcgcatgctgtggaaagaattctattggctgccactgatgacaagaaaactgaattccagaagaaaaagaatgccagagatgtcaaagaagggaaagaagaaatagatcacaacaaaacagggggcaccttttacaagatggtaagagatgataaaaccatctacttcagccctataagagtcacctttttaaaagaagaggtaaaaacaatgtacaaaaccaccatggggagtgatggcttcagcggactaaatcacataatgattgggcattcacagatgaatgatgtctgtttccaaagatcaaaggcactaaaaagagttggacttgacccttcattaatcagtacctttgcaggaagcacactccccagaagatcaggtgcaactggtgttgcgatcaaaggaggtggaactctagtggctgaagccattcgatttataggaagagcaatggcagacagagggctattgagagacatcaaagctaagactgcttatgaaaagattcttctgaatctaaaaaacaaatgctctgcgccccaacaaaaggctctagttgatcaagtgatcggaagtagaaatccagggatcgcagacattgaagacctaaccctgcttgctcgtagtatggtcgttgttaggccctctgtggcgagcaaagtagtgcttcccataagcatttacgccaaaatacctcaactagggttcaacgttgaagagtactctatggttgggtatgaagccatggctctttacaatatggcaacacctgtttccatattaagagtgggagatgatgcaaaagacaaatcacaattattcttcatgtcttgcttcggagctgcctatgaagacctgagagttttgtctgcattaacaggcacagagttcaagcctagatcagcattaaaatgcaagggtttccatgttccagcaaaggaacaggtggaaggaatgggggcagctctgatgtccatcaagctccagttttgggctccaatgaccagatctggggggaacgaagtaggtggagacggggggtctggccaaataagttgcagcccagtgtttgcagtagaaagacctattgctctaagcaagcaagctgtaagaagaatgctgtcaatgaatattgagggacgtgatgcagatgtcaaaggaaatctactcaagatgatgaatgactcaatggctaagaaagccaatggaaatgctttcattgggaagaaaatgtttcaaatatcagacaaaaacaaaaccaatcccgttgaaattccaattaagcaaaccatccccaatttcttctttgggagggacacagcagaggattatgatgacctcgattattaaagcaacaaaatagacactatgactgtgattgtttcaatacgtttggaatgtgggtgtttactcttattaaaataaatataaa

SEQ ID NO: 64 - DNA, 1516 bp, Influenza B
  source 1..1516: mol_type = other DNA
aaactgaggcaaataggccaaaaatgaacaatgctaccttcaactatacaaacgttaaccctatttctcacatcagggggagtattattatcactatatgtgtcagcttcattgtcatacttactatattcggatatattgctaaaattctcaccaacagaaataactgcaccaacaatgccattggattgtgcaaacgcatcaaatgttcaggctgtgaaccgttctgcaacaaaaggggtgacacttcttctcccagaaccagagtggacatacccgcgtttatcttgcccgggctcaacctttcagaaagcactcctaattagccctcatagattcggagaaaccaaaggaaactcagctcccttgataataagggaaccttttattgcttgtggaccaaaggaatgcaaacactttgctctaacccattatgcagcccaaccagggggatactacaatggaacaagaggagacagaaacaagctgaggcatctaatttcagtcaaattgggcaaaatcccaacagtagaaaactccattttccacatggcagcatggagcgggtccgcatgccatgatggtaaagaatggacatatatcggagttgatggccctgacaataatgcattgctcaaaataaaatatggagaagcatatactgacacataccattcctatgcaaacaacatcctaagaacacaagaaagtgcctgcaattgcatcgggggaaattgttatcttatgataactgatggctcagcttcaggtattagtgaatgcagatttcttaaaattcgagagggccgaataataaaagaaatatttccaacaggaagagtaaaacatactgaagaatgcacatgcggatttgccagcaataagaccatagaatgtgcctgtagagataacagttacacagcaaaaagaccctttgtcaaattaaacgtggagactgatacagcagaaataagattgatgtgcacagagacttatttggacacccccagaccagatgatggaagcataacagggccttgtgaatctaatggggacaaagggagtggaggcatcaagggaggatttgttcatcaaagaatggcatccaagattggaaggtggtactctcgaacgatgtctaaaactaaaaggatggggatgggactgtatgtcaagtatgatggagacccatgggctgacagtgatgcccttgctcttagtggagtaatggtttcaatggaagaacctggttggtactcctttggcttcgaaataaaagataagaaatgtgatgtcccctgtattggaatagagatggtacatgatggtggaaaagagacttggcactcagcagcaacagccatttactgtttaatgggctcaggacagctgctgtgggacactgtcacaggtgttgatatggctctgtaatggaggaatggttgagtctgttctaaaccctttgttcctattttgtttgaacaattgtccttactgaacttaattgtttctgaaa

SEQ ID NO: 65 - DNA, 1146 bp, Influenza B
  source 1..1146: mol_type = other DNA
aaaatgtcgctgtttggagacacaattgcctacctgctttcattgacagaagatggagaaggcaaagcagaactagcagaaaaattacactgttggtttggtgggaaagaatttgacctagactctgccttggaatggataaaaaacaaaagatgcttaactgatatacaaaaagcactaattggtgcctctatctgctttttaaaacccaaagaccaggaaagaaaaagaagattcatcacagagcccttatcaggaatgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttgtgcgagaaacaagcatcacattcacacagggctcatagcagagcagcgagatcttcagtgcctggagtgagacgagaaatgcagatggtctcagctatgaacacagcaaaaacaatgaatggaatgggaaaaggagaagacgtccaaaagctggcagaagagctgcaaagcaacattggagtgctgagatctcttggggcaagtcaaaagaatggggaaggaattgcaaaggatgtaatggaggtgctaaagcagagctctatgggaaattcagctcttgtgaagaaatatctataatgctcgaaccatttcagattctttcaatttgttcttttatcttatcagctctccatttcatggcttggacaatagggcatttgaatcaaataaaaagaggagtaaacatgaagatacgaataaaaagtccaaacaaagagacaataaacagagaggtatcaattttgagacacagttaccaaaaagaaatccaggccaaagaaacaatgaaggaagtactctctgacaacatggaggtattgagtgaccacatggtgattgaggggctttctgccgaagagataataaaaatgggtgaaacagttttggagatagaagaattgcattaaattcaatttttactgtatttcttaccatgcatttaagcaaattgtaatcaatgtcagcaaataaact

SEQ ID NO: 66 - DNA, 1055 bp, Influenza B
  source 1..1055: mol_type = other DNA
tcactggcaaacaggaaaaatggcgaacaacatgaccacaacacaaattgaggtgggtccgggagcaaccaatgccaccataaactttgaagcaggaattctggagtgctatgaaaggctttcatggcaaagagcccttgactaccctggacaagaccgcctaaacagactaaagagaaaattagagtcaagaataaagactcacaacaaaagtgagcctgaaagtaaaaggatgtcccttgaagagagaaaagcaattggagtaaaaatgatgaaagtactcctatttatgaatccgtctgctggaattgaagggtttgagccatactgtatgaaaagttcctcaaatagcaactgtacgaaatacaattggaccgattacccttcaacaccagggaggtgccttgatgacatagaagaagaaccagaggatgttgatggcccaactgaaatagtattaagggacatgaacaacaaagatgcaaggcaaaagataaaggaggaagtaaacactcagaaagaagggaagttccgtttgacaataaaaagggatatgcgtaatgtattgtccttgagagtgttggtaaacggaacattcctcaaacaccccaatggatacaagtccttatcaactctgcatagattgaatgcatatgaccagagtggaaggcttgttgctaaacttgttgctactgatgatcttacagtggaggatgaagaagatggccatcggatcctcaactcactcttcgagcgtcttaatgaaggacattcaaagccaattcgagcagctgaaactgcggtgggagtcttatcccaatttggtcaagagcaccgattatcaccagaagagggagacaattagactggtcacggaagaactttatcttttaagtaaaagaattgatgataacatattgttccacaaaacagtaatagctaacagctccataatagctgacatggttgtatcattatcattattagaaacattgtatgaaatgaaggatgtggttgaagtgtacagcaggcagtgcttgtgaatttaaaataaa